(19) United States
(12) Patent Application Publication
(10) Pub. No.: US 2010/0120727 A1
(43) Pub. Date: May 13, 2010

(54) Eflornithine Prodrugs, Conjugates and Salts, and Methods of Use Thereof

(75) Inventor: Feng Xu, Palo Alto, CA (US)

Correspondence Address: WILSON, SONSINI, GOODRICH & ROSATI, 650 PAGE MILL ROAD, PALO ALTO, CA, 94304-1050, US

(73) Assignee: Kyphia Pharmaceuticals, Inc., Palo Alto, CA (US)

(21) Appl. No.: 12/617,574

(22) Filed: Nov. 12, 2009

Related U.S. Application Data
(60) Provisional application No. 61/114,015, filed on Nov. 12, 2008.

(51) Int. Cl. A61K 31/60; C07C 229/26; C07C 315/00; C07C 69/76; A61K 31/195; C07C 229/00; A61K 31/22; A61P 35/04

(52) U.S. Cl. 514/165; 562/561; 562/429; 560/76; 514/564; 560/169; 514/551

(57) ABSTRACT

In one aspect, the present invention provides a composition of a covalent conjugate of an eflornithine analog with an anti-inflammatory drug. In another aspect, the present invention provides a composition of an eflornithine prodrug. In another aspect, the present invention provides a composition of an eflornithine or its derivatives aspirin salt. In another aspect, the present invention provides methods for treating or preventing cancer using the conjugates or salts of eflornithine analogs or eflornithine prodrugs.

### CROSS-REFERENCE

This application claims the benefit of U.S. Provisional Application No. 61/114,015, filed Nov. 12, 2008, which application is incorporated herein by reference.

### BACKGROUND OF THE INVENTION

Cancer, also known as malignant neoplasm, is a type of hyperproliferative disorder characterized by an abnormal growth of cells that display uncontrolled cell division, invasion and destruction of adjacent tissues, and sometimes metastasis to other locations in the body. There are more than 100 types of cancer, including breast cancer, skin cancer, lung cancer, colon cancer, prostate cancer, and lymphoma. Cancer may affect people at all ages, even fetuses, but the risk for most types of cancer increases with age. Cancers can affect all animals.

Cancer is influenced by multiple molecular mechanisms. Because of the complexity nature of each disease state, achieving cures with single agent has often met with limited success. Thus, combinations of agents have been frequently used in the treatment of cancers. It has been reported that there is strong correlation between the number of agents administered and cure rates for cancers such as acute lymphocytic leukemia and metastatic colorectal cancer (Frei, et al., Clin. Cancer Res. 1998, 4, 2027-2037; Fisher, M. D. Clin. Colorectal Cancer 2001, 1(2), 85-86).

α-Difluoromethylornithine (DFMO) or 2-(difluoromethyl)-DL-ornithine, also known as eflornithine, is an inhibitor of ornithine decarboxylase (ODC), the rate limiting enzyme of the polyamine biosynthetic pathway. As a result of this inhibition of polyamine synthesis, the compound is effective in preventing cancer formation in many organ systems, inhibiting cancer growth, and reducing tumor size. It also has synergistic action with other antineoplastic agents.

### SUMMARY OF THE INVENTION

In one aspect, the present invention provides a compound for treating or preventing cancer, the compound comprising a first moiety and a second moiety, the first moiety being covalently linked to the second moiety, wherein the first moiety is eflornithine or an analog or derivative of eflornithine, and the second moiety is a non-steroidal anti-inflammatory drug (NSAID). In some embodiments, the first moiety is eflornithine. In some embodiments, the second moiety is selected from the group consisting of aspirin, aceclofenac, acemethacin, alclofenac, amoxiprin, ampyrone, azapropazone, benorylate, bromfenac, choline and magnesium salicylates, choline salicylate, celecoxib, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, rofecoxib, salsalate, salicyl salicylate, salicylamide, sodium salicylate, sulindac, tiaprofenic acid, tolfenamic acid, tolmetin sodium, and valdecoxib. In some embodiments, the NSAID is sulindac. In some embodiments, the NSAID is aspirin. In some embodiments, the first and second moieties are linked via a covalent bond selected from the group consisting of an ester bond, an amide bond, an imine bond, a carbamate bond, a carbonate bond, a thioester bond, an acyloxycarbamate bond, an acyloxycarbonate bond, an acyloxythiocarbamate, a phosphate bond, a phosphoramidate and an acyloxyphosphate bond. In some embodiments, the compound further comprises a linker that covalently links the first moiety to the second moiety. In some embodiments, the linker is physiologically labile. In some embodiments, the cancer is adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia. In some embodiments, the cancer is a Ki-ras-dependent cancer. In some embodiments, the compound further comprises a third moiety that is ionically or covalently linked to the first moiety or second moiety of the compound. In some embodiments, the compound can be used in combination with at least one other therapeutic agent. In some embodiments, the other therapeutic agent is an antitumor alkylating agent, antitumor antimetabolite, antitumor antibiotics, plant-derived antitumor agent, antitumor organoplatinum compound, antitumor campthotecin derivative, antitumor tyrosine kinase inhibitor, monoclonal antibody, interferon, biological response modifier, hormonal anti-tumor agent, angiogenesis inhibitor, differentiating agent, or a pharmaceutically acceptable salt thereof. In some embodiments, compound can be used in combination with surgery, radiation therapy, chemotherapy, gene therapy, RNA therapy, adjuvant therapy, immunotherapy, nanotherapy or a combination thereof.

In some embodiments, the present invention provides a pharmaceutical composition for treating or preventing cancer, comprising the compound of claim 1 and a pharmaceutically acceptable carrier. In some embodiments, the present invention provides a kit for treating or preventing cancer in a subject, the kit comprising the compound of the invention or the pharmaceutical composition of the invention, and instructions for using the kit. In some embodiments, the subject is an animal, preferably a human. In some embodiments, the kit further comprises at least one other agent for use in the treatment of cancer, for reducing side effects induced by the compound of the invention, and/or for enhancing the therapeutic efficacy of the compound of the invention.

In another aspect, the present invention provides a method of treating or preventing cancer, the method comprising administering to a subject in need thereof a therapeutically effective amount of a compound, the compound comprising a first moiety and a second moiety, the first moiety being covalently linked to the second moiety, wherein the first moiety is eflornithine or an analog or derivative of eflornithine, and the second moiety is a non-steroidal anti-inflammatory drug (NSAID). In some embodiments, the first moiety is eflornithine. In some embodiments, the second moiety is selected from the group consisting of aspirin, aceclofenac, acemethacin, alclofenac, amoxiprin, ampyrone, azapropazone, benorylate, bromfenac, choline and magnesium salicylates, choline salicylate, celecoxib, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, rofecoxib, salsalate, salicyl salicylate, salicylamide, sodium salicylate, sulindac, tiaprofenic acid, tolfenamic acid, tolmetin sodium, and valdecoxib. In some embodiments, the NSAID is sulindac. In some embodiments, the NSAID is aspirin. In some embodiments, the first and second moieties are linked via a covalent bond selected from the group consisting of an ester bond, an amide bond, an imine bond, a carbamate bond, a carbonate bond, a thioester bond, an acyloxycarbamate bond, an acyloxycarbonate bond, a phosphate bond, a phosphoramidate bond and an acyloxyphosphate bond. In some embodiments, the compound further comprises a linker that covalently links the first moiety to the second moiety. In some embodiments, the linker is physiologically labile. In some embodiments, the cancer is adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia. In some embodiments, the cancer is a Ki-ras-dependent cancer. In some embodiments, the compound further comprises a third moiety that is ionically or covalently linked to the first moiety or second moiety of the compound. In some embodiments, the method further comprises administering to the subject at least one other therapeutic agent. In some embodiments, the other therapeutic agent is an antitumor alkylating agent, antitumor antimetabolite, antitumor antibiotics, plant-derived antitumor agent, antitumor organoplatinum compound, antitumor campthotecin derivative, antitumor tyrosine kinase inhibitor, monoclonal antibody, interferon, biological response modifier, hormonal anti-tumor agent, angiogenesis inhibitor, differentiating agent, or a pharmaceutically acceptable salt thereof. In some embodiments, the other therapeutic agent is administered prior to, concomitant with or subsequent to administering the compound. In some embodiments, the compound is administered in combination with surgery, radiation therapy, chemotherapy, gene therapy, RNA therapy, adjuvant therapy, immunotherapy, nanotherapy or a combination thereof. In some embodiments, the method further comprises administering to the subject a therapeutically effective amount of a pharmaceutical composition, the composition comprising the compound and a pharmaceutically acceptable carrier. In some embodiments, the subject is a mammal. In some embodiments, the subject is a human. In some embodiments, the compound is administered parenterally. In some embodiments, the compound is administered orally. In some embodiments, administering the compound or the pharmaceutical composition results in at least one less side effect as compared to administering the individual moiety alone. In some embodiments, administering the compound or the pharmaceutical composition results in enhanced therapeutic activity as compared to administering the individual moiety alone.

In another aspect, the present invention provides a compound of formula (IV) or (V) or its pharmaceutically acceptable salts

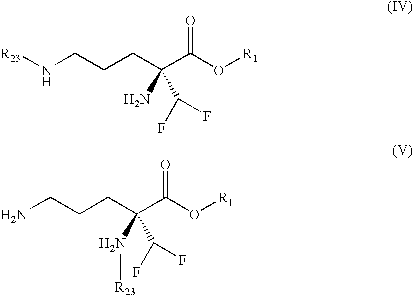

In some embodiments, the phosphoramidate group is cleaved in vivo. In some embodiments, the compound further comprises a pharmaceutically acceptable carrier.

In yet another aspect, the present invention provides a method of treating or preventing cancer, the method comprising administering to a subject in need thereof a therapeutically effective amount of a compound of formula (IV) or (V) or its pharmaceutically acceptable salts.

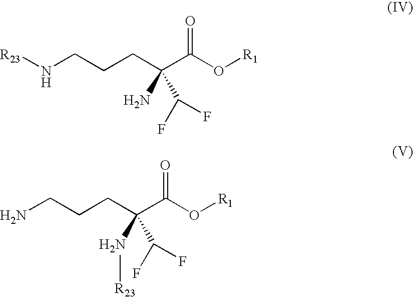

In some embodiments, the compound is administered orally. In some embodiments, the compound is administered parenterally. In some embodiments, the compound is administered in combination with an NSAID. In some embodiments, the NSAID is selected from the group consisting of aspirin, aceclofenac, acemethacin, alclofenac, amoxiprin, ampyrone, azapropazone, benorylate, bromfenac, choline and magnesium salicylates, choline salicylate, celecoxib, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, rofecoxib, salsalate, salicyl salicylate, salicylamide, sodium salicylate, sulindac, tiaprofenic acid, tolfenamic acid, tolmetin sodium, and valdecoxib. In some embodiments, the NSAID is sulindac. In some embodiments, the NSAID is aspirin. In some embodiments, the NSAID is administered prior to, concomitant with, or subsequent to administering the compound of formula (IV) or (V). In some embodiments, the subject is a mammal In some embodiments, the subject is a human. In some embodiments, the cancer is adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia. In some embodiments, the cancer is a Ki-ras-dependent cancer.

Also provided by the present invention is a composition for treating or preventing cancer comprising a salt of a mixture of eflornithine or an analog or derivative of eflornithine, and aspirin, for example, eflornithine ester aspirin salt.

In yet another aspect, the present invention provides a method of treating or preventing cancer comprising administering to a subject in need thereof a therapeutically effective amount of a salt of a mixture of eflornithine or an analog or derivative of eflornithine, and aspirin.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

In one aspect, the invention provides an eflornithine-NSAID conjugate comprising at least two moieties that are linked through a covalent bond. In some embodiments, the first moiety of the conjugate preferably comprises an eflornithine analog. In some embodiments, the second moiety of the conjugate comprises a non-steroidal anti-inflammatory drug (NSAID). In some embodiments, the NSAIDs of the second moiety of the conjugate include, but are not limited to, salicylates, arylakanoic acids, 2-arylpropionic acids (profens), N-arylanthranilic acids, pyrazolidine derivatives, oxicams, COX-2 inhibitors, sulphonanilides, or pharmaceutically acceptable salts thereof. In some embodiments, the NSAIDS include but are not limited to aspirin, choline and magnesium salicylates, choline salicylate, celecoxib, diclofenac potassium, diclofenac sodium, diclofenac sodium with misoprostol, diflunisal, etodolac, fenoprofen calcium, flurbiprofen, ibuprofen, indomethacin, ketoprofen, magnesium salicylate, meclofenamate sodium, mefenamic acid, meloxicam, nabumetone, naproxen, naproxen sodium, oxaprozin, piroxicam, rofecoxib, salsalate, sodium salicylate, sulindac, tolmetin sodium, valdecoxib, or a combination thereof.

In another aspect, the present invention also provides methods for synthesizing and producing the eflornithine prodrugs or eflornithine-NSAID conjugates.

In yet another aspect, the present invention includes methods for using the eflornithine prodrugs or eflornithine-NSAID conjugates to treat or prevent cancer, reduce adverse effect associated with treatment of cancer, and increase therapeutic efficacy of treatment of cancer.

Examples of eflornithine analogs include but are not limited to the following structures:

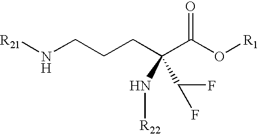

In some embodiments, eflornithine and NSAID are directly attached through amino groups or carboxylic acid group form the following linkers including but not limited to carboxylic ester, carbamate, carbonate, thiocarbonate, phosphate, phosphonate, phosphinic ester, phosphoramidate, thiophosphate, sulphonate, or sulphinic ester. Alternatively, the eflornithine analog and the NSAID are linked through a linker —X— that can be cleaved in vivo by chemical or enzymatic process.

In another aspect, the invention provides (D,L)-eflornithine-aspirin salt, (D)-eflornithine-aspirin salt and (L)-eflornithine-aspirin salt.

In another aspect, the present invention provides methods for synthesizing and producing the eflornithine-aspirin salt.

In yet another aspect, the present invention includes methods for using the eflornithine-aspirin salt to treat or prevent cancer, reduce adverse effect associated with treatment of cancer, and/or increase therapeutic efficacy of treatment of cancer.

The terms “DFMO,” and “Eflornithine” are used interchangeably and refer to the compound that is chemically designated as 2-(Difluoromethyl)-DL-ornithine, 2-(Difluoromethyl)ornithine, DL-α-difluoromethylornithine, N-Difluoromethylornithine, ornidyl, and αδ-Diamino-α-(difluoromethyl)valeric acid. In some embodiments, the present invention provides an eflornithine-NSAID conjugate of Formula (I), (II) & (III):

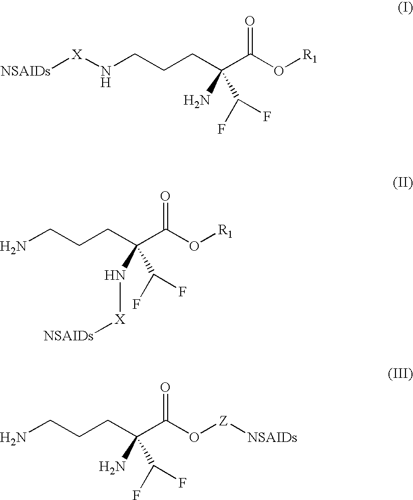

or a pharmaceutically acceptable salt, hydrate, solvate and isotopes, wherein:

X is a linker defined as a covalent bond between NSAID and an eflornithine analog. Examples of X include but are not limited to the following:

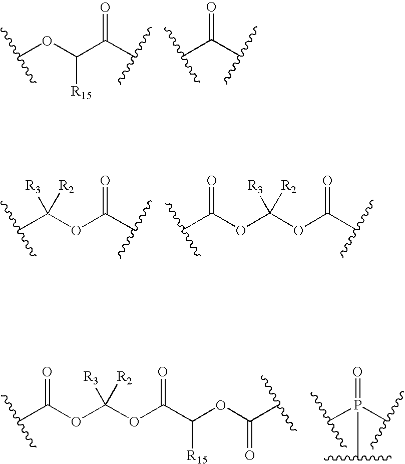

Z is defined as a covalent bond via a linker between a NSAID and an eflornithine analog that is selected from the group consisting of the following:

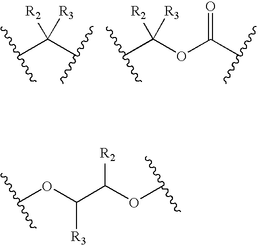

R₁ is independently selected from the group consisting of hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl and substituted heteroarylalkyl;

R₂ and R₃ are independently selected from the group consisting of hydrogen, alkyl, substituted alkyl, acyl, substituted alkoxycarbonyl, substituted alkoxycarbonyl, aryl, substituted aryl, arylalkyl, substituted arylalkyl, carbamoyl, cycloalkyl, substituted cycloalkyl, cycloalkoxycarbonyl, substituted cycloalkoxycarbonyl, heteroaryl, substituted heteroaryl, arylalkyl, substituted arylalkyl, heteroarylalkyl and substituted heteroarylalkyl;

R₁₅ is selected from the group consisting of hydrogen, alkyl, substituted alkyl, acyl, substituted alkoxycarbonyl, substituted alkoxycarbonyl, aryl, substituted aryl, arylalkyl, substituted arylalkyl, carbamoyl, cycloalkyl, substituted cycloalkyl, cycloalkoxycarbonyl, substituted cycloalkoxycarbonyl, heteroaryl, substituted heteroaryl, arylalkyl, substituted arylalkyl, heteroarylalkyl and substituted heteroarylalkyl;

R₂₁ and R₂₂ are independently selected from the group consisting of hydrogen, alkyl, substituted alkyl, acyl, aryl, substituted aryl, arylalkyl, substituted arylalkyl, cycloalkyl, substituted cycloalkyl, cycloalkoxycarbonyl, substituted cycloalkoxycarbonyl, heteroaryl, substituted heteroaryl, arylalkyl, substituted arylalkyl, heteroarylalkyl and substituted heteroarylalkyl;

The compounds of the invention can be identified either by their chemical structure and/or chemical name. In some embodiments, the compounds of the invention contain one or more chiral centers and/or double bonds and therefore, can exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. The compounds of the invention can also exist in several tautomeric forms including the enol form, the keto form and mixtures thereof. Accordingly, the chemical structures depicted herein encompass all possible tautomeric forms of the illustrated compounds. The compounds of the invention also include isotopically labeled compounds where one or more atoms have an atomic mass different from the atomic mass conventionally found in nature. Examples of isotopes that can be incorporated into the compounds of the invention include, but are not limited to, ²H, ³H, ³C, ¹⁴C, ¹⁵N, ¹⁸O, ¹⁷O, ³¹P, ³²P, ³⁵S, ¹⁸F and ³⁶Cl. Further, it should be understood, when partial structures of the compounds of the invention are illustrated, that brackets indicate the point of attachment of the partial structure to the rest of the molecule.

In still another aspect, the present invention provides a modified eflornithine analog or derivative which has improved oral bioavailability such that it can be administered orally to a subject. In some embodiments, the modified eflornithine analog is an eflornithine prodrug of Formula (IV) & (V):

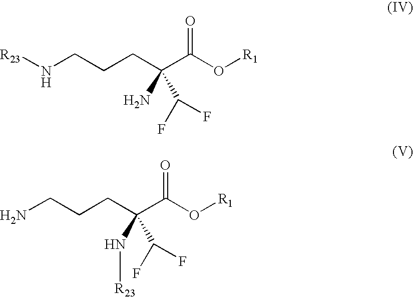

wherein R₂₃ is selected from the group consisting of hydrogen, R₂₄C(O)—, R₂₄OC(O)—, R₂₄C(S)—, R₂₄SC(O)—, and (R₂₄O)(R₂₄O)P(O)—.

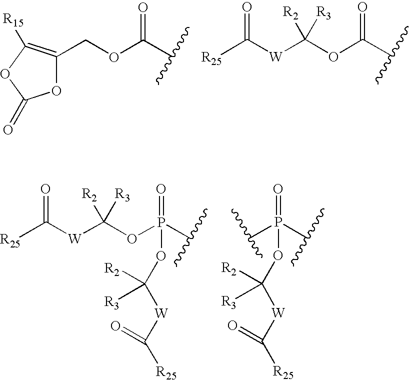

R₂₄ and R₂₅ are independently selected from the group consisting of alkyl, substituted alkyl, acyl, substituted acyl, aryl, substituted aryl, arylalkyl, substituted arylalkyl, cycloalkyl, substituted cycloalkyl, cycloalkoxycarbonyl, substituted cycloalkoxycarbonyl, heteroaryl, substituted heteroaryl, arylalkyl, substituted arylalkyl, heteroarylalkyl and substituted heteroarylalkyl;

W can be —O—, or —NH—;

A “prodrug” as used herein generally refers to a precursor of a drug. A prodrug typically undergoes chemical or biological conversion before becoming an active pharmacological agent. The phosphoramindate group is cleaved upon in vivo administration of the eflornithine prodrug, releasing the active form of eflornithine in vivo. The phosphoramidate group released from the eflornithine prodrug is typically non-toxic when the eflornithine prodrug is administered to a mammal at a therapeutically effective dosage. The eflornithine prodrugs of formula (IV) or (V) have increased aqueous solubility suitable for oral formulation and circumvent the inter-patient variability of eflornithine derivative, oral bioavailability due to efflux and polymorphanism of enzymatic metabolism. In some embodiments, the aqueous solubility of the eflornithine prodrug is increased by greater than about 5%, 10%, 15%, 20%, 30%, 35%, 40%, 45%, 50%, 60%, 70%, or 80%, as compared to that of eflornithine or any analog or derivative thereof. In some embodiments, the modified eflornithine derivative of the invention, e.g. the eflornithine prodrug, is covalently conjugated to an NSAID to form the eflornithine-NSAID conjugate as described herein. Some embodiments further provide a pharmaceutical composition of the eflornithine prodrug.

In another aspect, the invention provides a method of treating cancer comprising administering to a subject in need thereof an eflornithine prodrug of formula (IV) or (V), or a pharmaceutical composition thereof. In some embodiments, the eflornithine prodrug of formula (IV) or (V) is administered orally. In some embodiments, the eflornithine prodrug of formula (IV) or (V) is administered alone to a subject. In some embodiments, the eflornithine prodrug of formula (IV) or (V) is administered in combination with an NSAID. The NSAID can be any NSAID described herein. In some embodiments, the eflornithine prodrug and the NSAID are administered in the same formulation. In other embodiments, the eflornithine prodrug and the NSAID are administered in different formulations. The eflornithine prodrug of formula (IV) or (V) can be administered prior to, concurrently with, or subsequent to the administration of the NSAID.

The compositions and methods of the present invention provide several advantages over the current treatment regimens. In some embodiments, the eflornithine-NSAID conjugate have improved physiochemical properties (e.g. solubility, absorption, metabolism, etc.).

In other embodiments, the eflornithine-aspirin salt or eflornithine ester-aspirin salt have improved physiochemical properties (e.g. solubility, absorption, metabolism, etc.). It can provide a more predictive dosing regimen and achieve a better therapeutic outcome among cancer patients. In other embodiments, by optimizing the linker between the two moieties of the conjugate, the rate of releasing the cytotoxic agents (e.g. eflornithine and an NSAID) can be optimized. As a result, some of the gastrointestinal (GI) side effects can be reduced in comparison to the administration of the parent drugs. In some embodiments, the eflornithine-aspirin salt targets two or more biological targets that are relevant for cancer treatment and prevention. Additionally, the clinical efficacy can also be improved by potentially releasing two synergistic cytotoxic agents simultaneously in cancer cells.

The chemical structures of eflornithine-aspirin salt and eflornithine ester-aspirin salt are shown below:

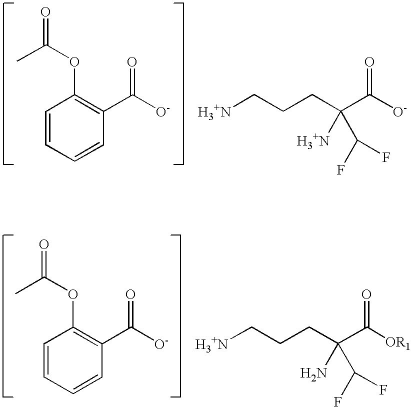

In some embodiments, the salt composition of the present invention contains different molar ratios of eflornithine or an eflornithine derivative to aspirin. The molar ratio of eflornithine or an eflornithine derivative, e.g. eflornithine ester, to aspirin can be in the range of about 3:1 to 1:3. In one embodiment, the molar ratio of eflornithine or eflornithine ester to aspirin in a salt mixture is about 1:1. In another embodiment, the molar ratio of eflornithine or eflornithine ester to aspirin in a salt mixture is about 2:1.

### I. First Moiety: Eflornithine Analog

In one aspect, the present invention provides a composition for treating cancer or a hyperproliferative disease. In some embodiments, the first moiety of the composition is an eflornithine analog.

Eflornithine or difluoromethylornithine (DFMO) is an irreversible inhibitor of ornithine decarboxylase (ODC) and potentially can be given continuously with significant anti-tumor effects. This drug is relatively non-toxic at low doses of 0.4 gr/m²/day to humans while producing inhibition of putrescine synthesis in tumors. Studies in a rat-tumor model demonstrate that DFMO infusion can produce a 90% decrease in tumor putrescine levels without suppressing peripheral platelet counts. Eflornithine has also been found to be highly effective in African trypanosomiasis (sleeping sickness). A recent study indicates that, when combined with sulindac (an anti-inflammatory drug), DFMO significantly reduces the risk of recurring colorectal polyps.

Although DFMO can effectively block tumor putrescine biosynthesis, the resultant antitumor effect is cytostasis and not cytotoxicity. For example, DFMO reduces the growth rate of an MCA sarcoma but does not produce tumor regression. This finding is consistent with other reports showing that DFMO is a cytostatic agent. However, studies indicate that a significant role exists for DFMO agents, permitting the future development of combination chemotherapeutic regimens which incorporate DFMO.

DFMO and its use in the treatment of benign prostatic hypertrophy are described in two patents, U.S. Pat. Nos. 4,413,141, and 4,330,559. U.S. Pat. No. 4,413,141 describes DFMO as being a powerful inhibitor of ODC, both in vitro and in vivo. Administration of DFMO induces a decrease in putrescine and spermidine concentrations in cells in which these polyamines are normally actively produced. Additionally, DFMO has been shown to be capable of slowing neoplastic cell proliferation when tested in standard tumor models. U.S. Pat. No. 4,330,559 describes the use of DFMO and DFMO derivatives for the treatment of benign prostatic hypertrophy. Benign prostatic hypertrophy, similar to many disease states characterized by rapid cell proliferation, is accompanied by abnormal elevation of polyamine concentrations. The treatment described within this reference can be administered to a patient either orally, or parenterally.

Although inhibition of polyamine synthesis has proven to be generally ineffective as an anticancer strategy in clinical trials, it is a potent cancer chemoprevention strategy in preclinical and clinical studies, specially, in combination with anti-inflammatory drugs (Meyskens, F L Jr. Clin. Cancer Res. 1999, 5(5), 945-951; Gerner, E. W. Nat. Rev. Cancer 2004, 4(10), 781-792; Reddy B. S. Environ Mol. Mutagen. 2004, 44(1), 26-35; Raul F. Biochem. Soc. Trans. 2007, 35, 353-355; Presentation by Dr. Frank Meyskens from UC Irvine on Apr. 14, 2008 at annual meeting of American Association for Cancer Research in San Diego).

A significant problem with DFMO, i.e. eflornithine, is rapid systemic clearance, which consequently requires frequent dosing or continuous infusion to maintain a therapeutic or prophylactic concentration in the systemic circulation (Na-Bangchang K, et. al. Eur. J. Clin. Pharmacol. 2004, 60, 269-278). For example, dosing regimens for treating African tryponosomiasis or cancer chemoprevention therapy consists of 100 mg/kg at intervals of 6 hours for 14 days of DFMO given as short infusion or three to four time daily oral administrations.

Sustained released formulations are a conventional solution to the problem of rapid systemic clearance, as is well known to those skills in the art (See, e.g., “Remington's Pharmaceutical Sciences,” Philadelphia College of Pharmacy and Science, 17^{th} Edition, 1985). Osmotic delivery systems are also recognized methods for sustained drug delivery (see, e.g., Verma et al., Drug Dev. Ind. Pharm., 2000, 26, 695-708). DFMO is not absorbed via the large intestine. Rather, it is typically absorbed in the small intestine by perhaps amino acid transporter mechanism. The rapid passage of conventional dosage forms through the proximal absorptive region of the gastrointestinal tract has prevented the successful application of sustained released technologies. Thus, there is a need for effective sustained release of eflornithine analogs to minimize increased dosing frequency due to rapid systemic clearance of this drug.

In one aspect, the present invention provides an eflornithine-NSAID conjugate which has a new chemical entity that has several advantages over the existing treatment regimens. First, these eflornithine-NSAID conjugates are typically labile in vivo, cleaved by either enzymatic or chemical pathway, to generate substantial quantities of eflornithine analogs and the NSAIDs upon reaching the systemic circulation and tumor cells. Second, the eflornithine analogs, and the other NSAIDs, upon cleavage of the eflornithine-NSAID conjugates, typically target two or more biological targets that are relevant for cancer treatment. The linkers released from the conjugates are typically non-toxic when administered to a mammal with dosing regimens typically comparable to the co-administration of eflornithine analogs and the NSAIDs. Third, in some embodiments, the NSAIDs which are linked to eflornithine derivatives are hydrophilic. As a result, the final eflornithine-NSAID conjugates are more hydrophilic than the parent NSAID analogs. Therefore, the final eflornithine-NSAID conjugates can have higher water solubility/dissolution rate than the parent NSAID derivatives.

### II. Second Moiety: NSAID

NSAIDs are anti-inflammatory agents that are not steroids. In addition to anti-inflammatory actions, they have analgesic, antipyretic, and platelet-inhibitory actions. They are used primarily in the treatment of chronic arthritic conditions and certain soft tissue disorders associated with pain and inflammation. They act by blocking the synthesis of prostaglandins by inhibiting cyclooxygenase, which converts arachidonic acid to cyclic endoperoxides, precursors of prostaglandins. Inhibition of prostaglandin synthesis accounts for their analgesic, antipyretic, and platelet-inhibitory actions; other mechanisms can contribute to their anti-inflammatory effects. Certain NSAIDs also can inhibit lipoxygenase enzymes or phospholipase C or can modulate T-cell function (AMA Drug Evaluations Annual, 1994, p 1814-5). Examples of NSAIDs include, but are not limited to, aspirin, aceclofenac, acemethacin, alclofenac, amoxiprin, ampyrone, azapropazone, benorylate, bromfenac, choline and magnesium salicylates, choline salicylate, celecoxib, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, rofecoxib, salsalate, salicyl salicylate, salicylamide, sodium salicylate, sulindac, tiaprofenic acid, tolfenamic acid, tolmetin sodium, valdecoxib, or a combination thereof.

The NSAIDs, including but not limited to aspirin, ibuprofen, piroxicam (Reddy et al., Cancer Res. 1990, 2562-2568; Singh et al., Carcinogenesis, 1994, 1317-1323), indomethacin (Narisawa, Cancer Res., 1981, 1954-1957), and sulindac (Piazza et al., Cancer Res. 1997, 2909-2915; Rao et al., Cancer Res. 1995, 1464-1472), effectively inhibit colon carcinogenesis in the AOM-treated rat model. NSAIDs also inhibit the development of tumors harboring an activated Ki-ras (Singh and Reddy, Annals of the New York Academy of Science, 1995, 205-209). Without wishing to be bound by any theory, NSAIDs appear to inhibit carcinogenesis via the induction of apoptosis in tumor cells (Bedi et al., Cancer Res., 1995, 1811-1816; Lupulescu, Prostaglandins, Leukotrienes, and Essential Fatty Acids, 1996, 83-94; Piazza et al., Cancer Res., 1995, 3110-3116; Piazza et al., Cancer Res. 1997, 2452-2459). A number of studies suggest that the chemopreventive properties of the NSAIDs, including the induction of apoptosis, are a function of their ability to inhibit prostaglandin synthesis (reviewed in DuBois et al., Gastroenterology, 1996, 773-791; Lupulescu, 1996; Vane and Botting, Sem. In Arthritis and Rheumatism, 1997, 2-10). Recent studies, however, indicate that NSAIDs can act through both prostaglandin-dependent and -independent mechanisms (Alberts et al., J. Cell. Biochem. Supp., 1995, 18-23; Piazza et al., Cancer Res., 1997, 3110-3116; Thompson et al., J. Nat'l Cancer Inst., 1995, 1255-1260; Hanif, et al., Biochemical Pharmacology, 1996, 237-245). Sulindac sulfone, a metabolite of the NSAID sulindac, lacks COX-inhibitory activity yet induces apoptosis in tumor cells (Piazza et al., Cancer Res., 1995, 3110-3116; Piazza et al., Cancer Res., 1997, 2452-2459) and inhibits tumor development in several rodent models of carcinogenesis (Thompson et al., 1995; Piazza et al., Cancer Res., 1995, 3110-3116). Preclinical and clinical studies have shown a benefit of NSAID use in reducing risk of developing several types of cancer, including but not limited to colorectal cancer, breast cancer and ovarian cancer. (Cha Y I, et. al. Annu Rev Med. 2007; 58:239-52).

The two main adverse drug reactions associated with NSAIDs relate to gastrointestinal (GI) effects and renal effects of the agents. These effects are dose-dependent, and in many cases severe enough to pose the risk of ulcer perforation, upper gastrointestinal bleeding, and death, limiting the use of NSAID therapy. An estimated 10-20% of NSAID patients experience dyspepsia, and NSAID-associated upper gastrointestinal adverse events are estimated to result in 103,000 hospitalizations and 16,500 deaths per year in the United States, and represent 43% of drug-related emergency visits.

NSAIDs induce apoptosis in both colon tumor cell lines and animal tissues, and appear to inhibit Ki-ras activation in tumors. However, the activation of Ki-ras has not yet been investigated as a mechanism of NSAID-mediated cytotoxicity. It also is not known if such cytotoxicity is dependent on the anti-inflammatory properties of the NSAIDs. The NSAID sulindac, which also inhibits Ki-ras activation, is metabolized to two different molecules which differ in their ability to inhibit COX, yet both are able to exert chemopreventive effects via the induction of apoptosis Sulindac sulfone lacks COX-inhibitory activity, and most likely facilitates the induction of apoptosis in a manner independent of prostaglandin synthesis. Indeed, several published clinical studies have demonstrated that using either DFMO alone or in combination with NSAIDs such as sulindac appears to be effective as chemoprevention agents (Gerner E. W. et al. Amino Acids 2007, 33(2), 189-195; Simoneau A. R. et al. Cancer Epidemiol Biomarkers Prey. 2008, 17(2), 292-299).

In some embodiments, the present invention provides a composition that can reduce the side effects associated with the NSAIDs and increase the therapeutic efficacy of the eflornithine treatment.

### Sulindac

Sulindac is a non-steroidal, anti-inflamatory indene derivative with the following chemical designation; (Z)-5-Fluoro-2-methyl-1-((4 (methylsulfinyl)phenyl)methylene)1H-indene-3-acetic acid (Physician's Desk Reference, Medical Economics Data, Montville, N.J., 1745-1747, 1999). Available evidence indicates that the sulfide derivative is the biologically activite compound. Based on this, sulindac is defined as a prodrug, and appears to be inactive or relatively weak in many tests where little or no metabolism can occur. Sulindac (Clinoril®) is available as 150- and 200-mg tablets. The most common dosage for adults is 150 to 200 mg twice a day, with a maximal daily dose of 400 mg. After oral administration, about 90% of the drug is absorbed. Peak plasma levels are achieved in about 2 hours in fasting patients and 3 to 4 hours when administered with food. The mean half-life of sulindac is 7.8 hours: the mean half-life of the sulfide metabolite is 16.4 hours. U.S. Pat. Nos. 3,647,858 and 3,654,349 cover preparations of sulindac.

Sulindac is currently indicated for the acute and long-term relief of signs and symptoms of osteoarthritis, rheumatoid arthritis, ankylosing spondylitis, acute gout, and acute painful shoulder. The analgesic and antiinflammatory effects exerted by sulindac (400 mg per day) are comparable to those achieved by aspirin (4 g per day), ibuprofin (1200 mg per day), indomethacin (125 mg per day), and phenylbutazone (400 to 600 mg per day). Side effects of sulindac include mild gastrointestinal effects in nearly 20% of patients, with abdominal pain and nausea being the most frequent complaints. CNS side effects are seen in up to 10% of patients, with drowsiness, headache, and nervousness being those most frequently reported. Skin rash and pruritus occur in 5% of patients. Chronic treatment with sulindac can lead to serious gastrointestinal toxicity such as bleeding, ulceration, and perforation.

The potential use of sulindac for chemoprevention of cancers, and in particular colorectal polyps, has been well studied. Two recent U.S. Pat. Nos. 5,814,625 and 5,843,929, detail potential chemopreventive uses of sulindac in humans. Doses of sulindac claimed in U.S. Pat. No. 5,814,625 range from 10 mg to 1500 mg per day, with preferred doses of 50 mg to 500 mg per day. However, at the higher doses, the biggest problem with the use of sulindac as a single agent in chemoprevention is its well-known toxicities and moderately high risk of intolerance.

### Aspirin

Aspirin has anti-inflammatory and antipyretic properties and acts as an inhibitor of cyclooxygenase which results in the inhibition of the biosynthesis of prostaglandins. Aspirin also inhibits platelet aggregation and is used in the prevention of arterial and venous thrombosis. (From Martindale, The Extra Pharmacopoeia, 30th ed, p 5). An example of eflornithine-aspirin conjugate of the invention is shown in Example 1.

Aspirin (acetylsalicylic acid) is one of the most widely used drugs in the world. It has a number of important therapeutic utilities, such as analgesic, antipyretic, anti-inflammatory. Aspirin also inhibits the platelet aggregation that potentially contributes to heart attack and stroke (Hennekens, C. H. et al. N. Engi. J. Med. 1989, 321, 129; Gossel, T. A. U.S. Pharmacist, February, 1988, p 34). It has been found to be effective to prevent various cancers, such as colon rectum cancer, prostate cancer and esophageal cancer, etc (Gabriel Kune, International Journal of Epidemiology 2007, Oct. 5^{th}, 1-3; Baron, J. A. et al. N. Engl. J. Med. 2003, 348, 891; Benamouzig, R. et al. Gastroenterology 2003, 125, 328-336; Sandler, R. et al. N. Engl. J. Med. 2003, 348, 883). The therapeutic utilities of aspirin continue growing even after it was first synthesized over 100 years ago.

Despite the convincing evidence of using aspirin in cancer prevention, a number of side effects are associated with the use of aspirin, most notably GI disturbances such as dyspepsia, gastroduodenal bleeding, gastric ulceration and gastritis. Aspirin has very low solubility in water and can stay in the GI tract for a long time, thus causing gastric mucosal cell damage. Stable salt of O-acetylsalicylic with basic amino acids has been studied for their use in treating various of diseases (Franckowiak, G. et. al. U.S. Pat. No. 6,773,724). The good solubility of these O-acetylsalicylates is an advantage compared with O-acetylsalicylic acid. In the case of relatively long oral administration, good tolerability of the O-acetylsalicylates is desirable.

Given the complexity and heterogenecity of carcinogenic mechanisms, the rationale for co-administering two or more agents with different modes of action showing synergistic effects while producing minimal toxicity seems compelling. In this context, the application of DFMO-drug combinations in chemoprevention are very attractive. Studies in rodent models have shown that combination chemoprevention strategies are often more effective than those using individual agents (Sporn M B. Nature 1980, 287, 107-8; Torrance C. J., et al. Nat Med 2000, 6, 1024-8). Difluoromethylornithine (DFMO) has been identified as a potent inhibitor of intestinal and colon carcinogenesis in animal models, especially in combination with nonsteroidal anti-inflammatory drugs (Nigro N. D. et al., J Natl Cancer Inst 1986, 77, 1309-13; Meyskens F L Jr, Clin Cancer Res 1999, 5, 945-51). Low doses of several NSAIDs, including aspirin and DFMO, administered in combination have been shown to be more effective in inhibiting chemically induced colon adenocarcinomas in rats than are high doses of these agents given individually (Reddy, B. S. et al. Curr. Gastroenterol. Rep. 2005, 7, 389-395). Aspirin and other NSAIDs, by inactivating cyclo-oxygenases and by lowering mucosal prostaglandin concentrations, can have a meaningful impact on polyp development by decreasing both size and number of polyps. Additionally, DFMO-aspirin combination given in the drinking water to rats (equivalent to a dose of 0.4 g/m² per day in human) starting 5 months after the initiation of colorectal carcinogenesis by a chemical carcinogen, prevented intestinal tumor formation during the 3 months of follow up by computed tomographic colonography in living rats. During the same period all non-treated rats exhibited colorectal tumor formation. Furthermore, a complete inactivation of ODC associated with a 20% reduction of the polyamine contents, and a 50% decrease of prostaglandin E₂ were observed in the colonic mucosa of the DFMO-aspirin treated rats (Raul F. Biochemical Society Transactions 2007, 35, 353-355).

DFMO and the nonsteroidal anti-inflammatory drug sulindac also interact additively to prevent the growth and viability of human colon cancer cells (Lawson K R, et al. Cancer Epidemiol Biomarkers Prey 2000, 9, 1155-62). Recently, the results of a phase III clinical chemoprevention trial evaluating the combination of DFMO and sulindac for the prevention of colon polyp recurrence are also reported. (Meyskens, F L Jr. Clin. Cancer Res. 1999, 5(5), 945-951; Gerner, E. W. Nat. Rev. Cancer 2004, 4(10), 781-792; Reddy B. S. Environ Mol. Mutagen. 2004, 44(1), 26-35; Raul F. Biochem. Soc. Trans. 2007, 35, 353-355; Presentation by Dr. Frank Meyskens from UC Irvine on Apr. 14, 2008 at annual meeting of American Association for Cancer Research in San Diego).

### III. Synthesis of Eflornithine Prodrugs and Eflornithine-NSAID Conjugates

Those of skill in the art will appreciate that compounds of Formulae (I)-(XIV) share certain structural features in common These compounds are DFMO (eflornithine) analogs to which promoieties or NSAIDs have been attached.

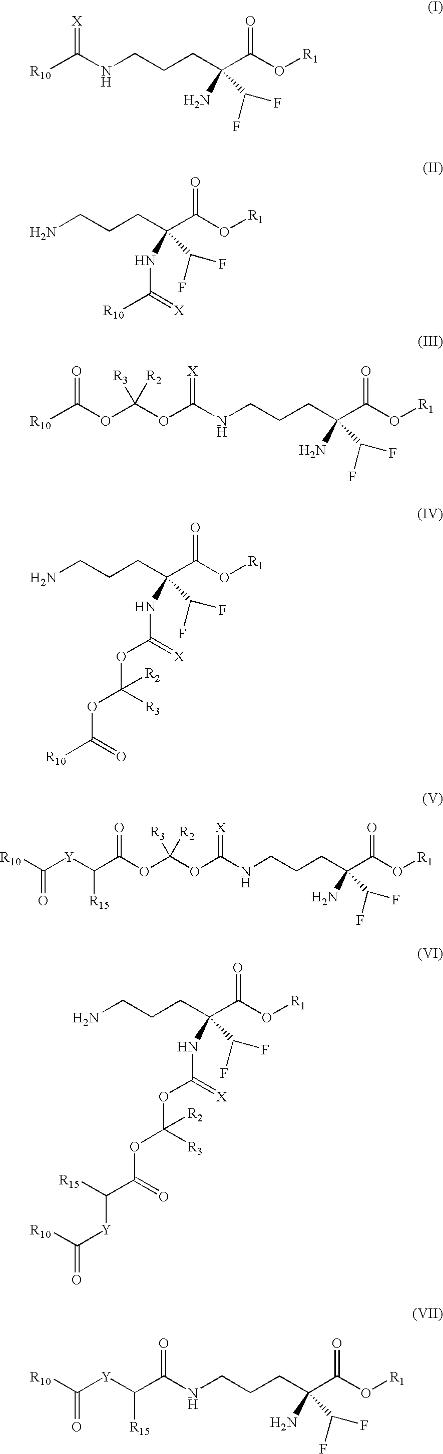

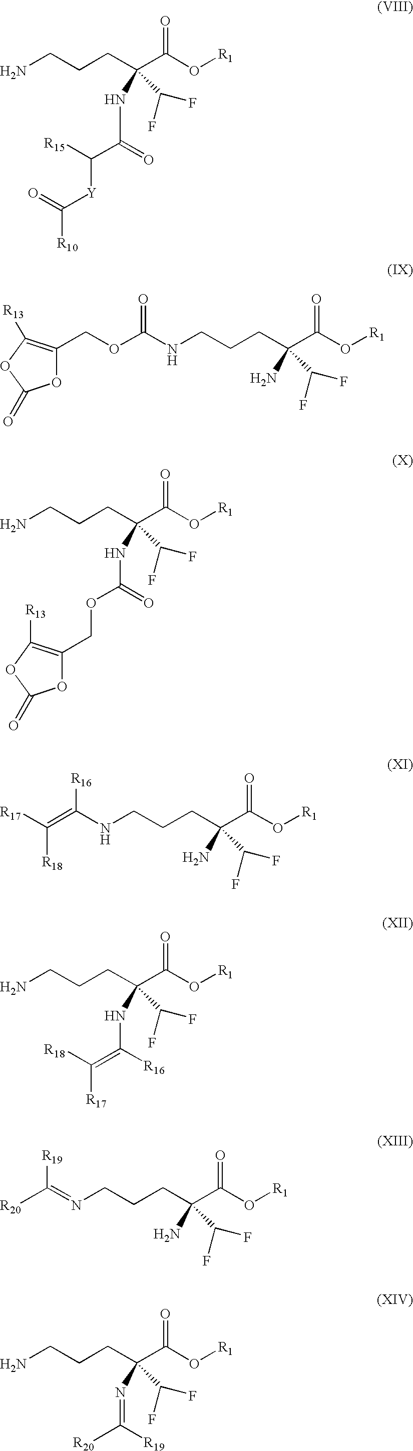

Wherein X=—O—, —S—; Y=—O—, —NH—, —S—;

In some embodiments, the eflornithine prodrug or eflornithine-NSAID conjugate of the invention is obtained via the synthetic methods illustrated in Schemes 1-10. Those of skill in the art will appreciate that a preferred synthetic route to the eflornithine prodrug or eflornithine-NSAID conjugate of the invention consists of attaching promoieties or an NSAID including but not limited to acetylsalicylic acid, diflunisal, ethenzamide, faislamine, diclofenac, aceclofenac, acemetacin, alclofenac, bromfenac, etodolac, indometacin, nabumetone, oxametacin, proglumetacin, sulindac, tolmetin, ibuprofen, alminoprofen, carprofen, dexibuprofen, dexketoprofen, fenbufen, fenoprofen, flunoprofen, flurbiprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, poroxicam, lornoxicam, meloxicam, tenoxicam. Several methods have been described in the art for the synthesis of eflornithine (See, e.g., Osipov, S, N. et al. Tetrahedron Lett. 1997, 38, 5965-5966; U.S. Pat. No. 6,730,809). Other methods are known in the art for synthesizing eflornithine, which are readily accessible to the skilled artisan. The promoieties or conjugation of NSAIDs to eflornithine described herein, are known in the art and can be prepared according to the known procedures. The art of introducing promoieties or attaching an NSAID containing various functional groups (e.g. carboxylic acid, hydroxyl, ketone, thiol, amine, amide, sulfonamide) to eflornithine is described by established procedures (See e.g., Green et al., “Protective Groups in Organic Chemistry”, (Wiley, 2^{nd} ed. 1991); Harrison et al., “Compendium of Synthetic Organic Methods”, Vols. 1-8 (John Wiley and Sons, 1971-1996); “Beilstein Handbook of Organic Chemistry,” Beilstein Institute of Organic Chemistry, Frankfurt, Germany; Feiser et al., “Reagents for Organic Synthesis,” Volumes 1-17, Wiley Interscience; Trost et al., “Comprehensive Organic Synthesis,” Pergamon Press, 1991; “Theilheimer's Synthetic Methods of Organic Chemistry,” Volumes 1-45, Karger, 1991; March, “Advanced Organic Chemistry,” Wiley Interscience, 1991; Larock “Comprehensive Organic Transformations,” VCH Publishers, 1989; Paquette, “Encyclopedia of Reagents for Organic Synthesis,” John Wiley & Sons, 1995, Bodanzsky, “Principles of Peptide Synthesis,” Springer Verlag, 1984; Bodanzsky, “Practice of Peptide Synthesis,” Springer Verlag, 1984).

Accordingly, starting materials useful for preparing compounds of the invention and intermediates thereof are commercially available or can be prepared by well-known synthetic methods. Other methods for synthesis of the prodrug or eflornithine-NSAID conjugates described herein are either described in the art or will be readily apparent to the skilled artisan in view of the references provided above and can be used to synthesize the eflornithine prodrug or eflornithine-NSAID conjugates of the invention. Accordingly, the methods presented in the schemes herein are illustrative rather than comprehensive.

In any of the Schemes below, after the amino group of eflornithine has been linked with an NSAID or other protecting group, the carboxylic acid group can be converted to an ester or thioester by many synthetic methods, which are well-known to the skilled artisan. The second amino group can also be converted to an amide or carbamate derivatives by synthetic methods, which are well-known to the skilled artisan. In one preferred embodiment, eflornithine can be reacted with an alcohol or thiol in the presence of a coupling reagent (e.g., carbodiimide and dimethylaminopyridine) to provide the ester or thioester. In another preferred embodiment, eflornithine can be reacted with an alkyl halide in the presence of a base to yield the ester. Other methods for converting eflornithine to esters or thioesters are well within the purview of the skilled artisan in view of the references provided herein.

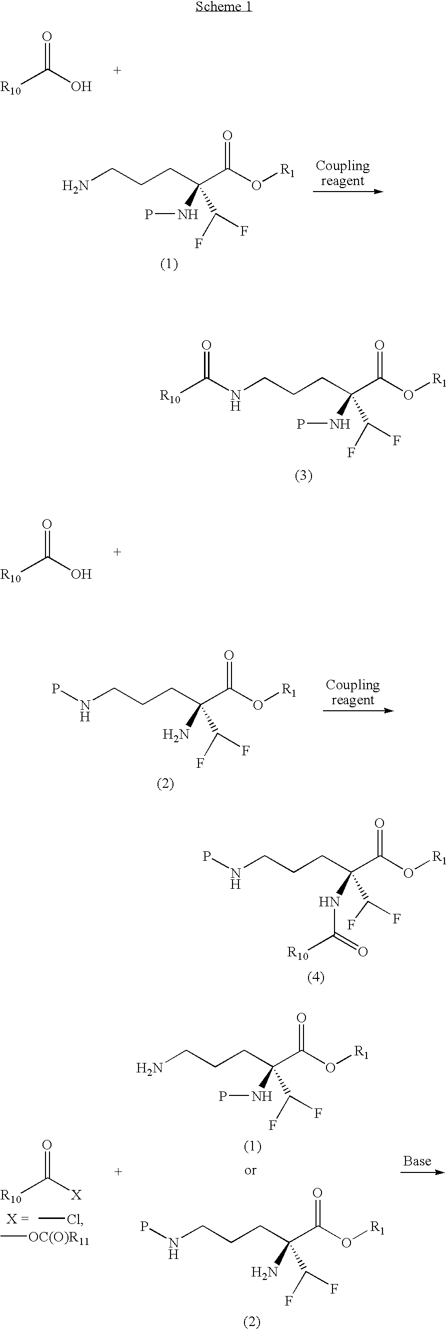

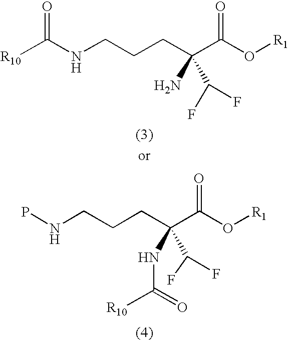

As illustrated in Scheme 1, promoieties containing carboxylic acids or NSAIDs containing carboxylic acids can be directly coupled to the terminal amino groups of eflornithine derivative (1) or (2) to provide adducts (3) or (4). Reagents for effecting this reaction are well known to the skilled artisan and include, but are not limited to, carbodiimides, aminium salts, and phosphonium salts. Alternatively, reaction of carboxylic acid can be activated by forming acyl chlorides, anhydrides followed with eflornithine derivative (1) or (2) in the presence of a base (e.g., hydroxide, tertiary amines, etc.) can be used to synthesize (3) or (4).

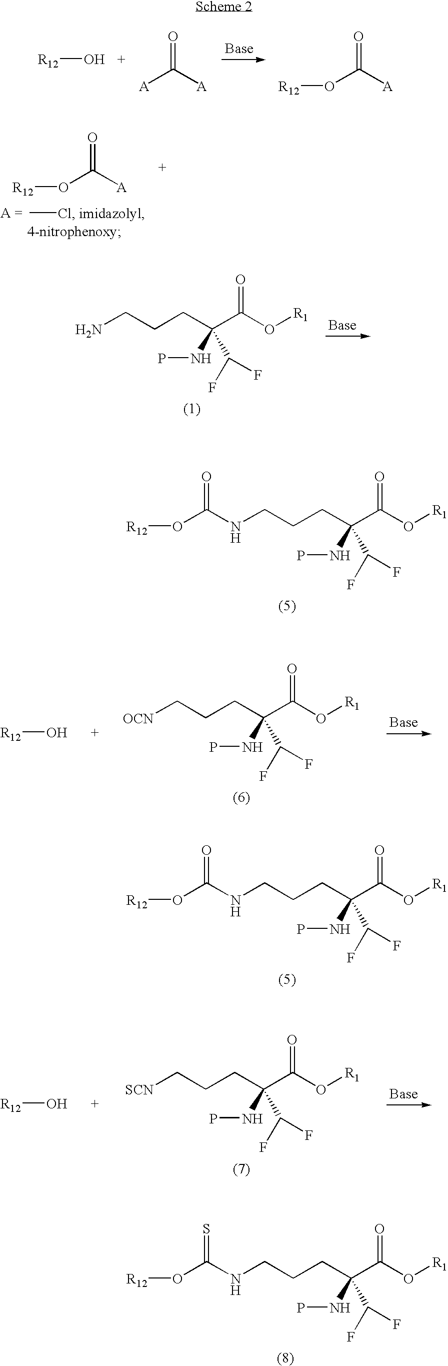

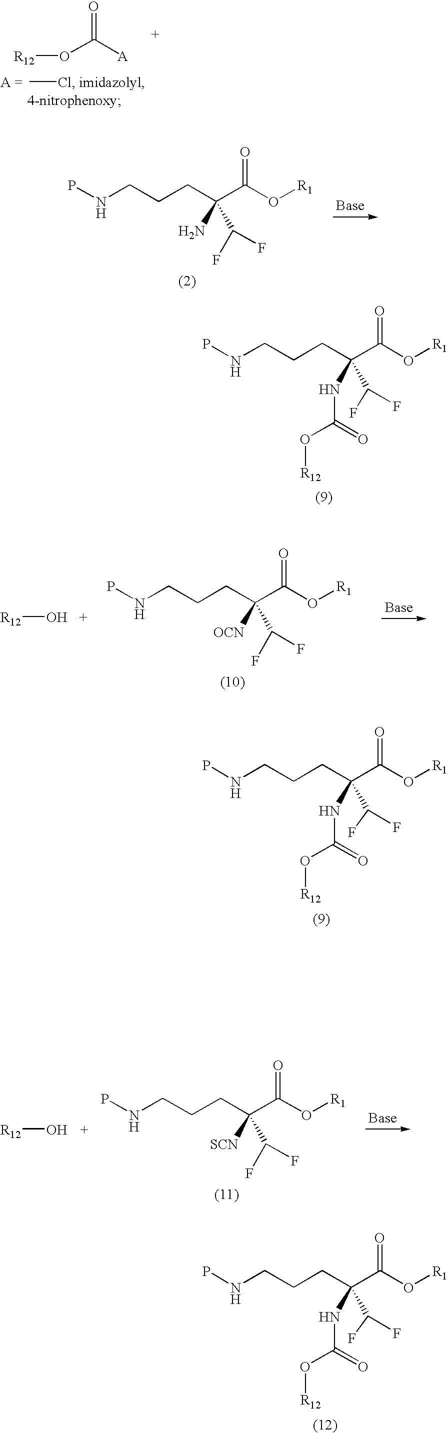

As illustrated in Scheme 2, eflornithine derivatives (1) or (2) can be converted to carbamate (5) and (9) by treatment with various carbonic acid derivatives (can be derived from NSAID analogs) in the presence of a base (e.g. hydroxide, tertiary amines, etc.). Alternatively, the well-known addition of alcohols to isocyanates (6) and (10) or thioisocyanates (7) and (11) can be used to synthesize these analogs.

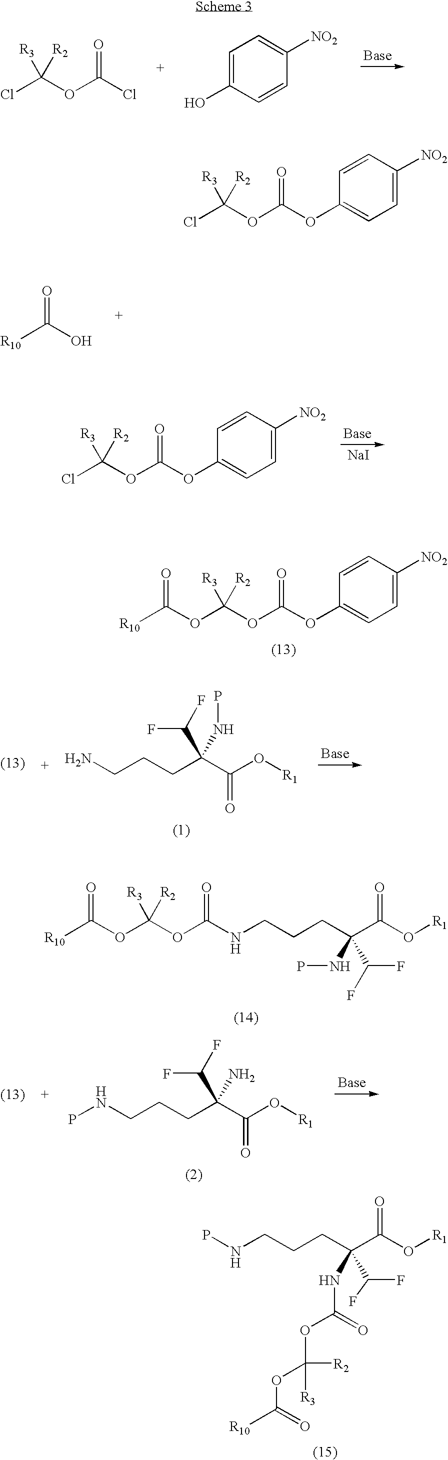

One method for synthesis of eflornithine-NSAID conjugate or eflornithine prodrug of formula (13) or (15) is illustrated in Scheme 3. Chloroformate is first treated with an aromatic leaving group such as p-nitrophenol in the presence of a base to provide p-nitrophenylcarbonate which is then reacted with carboxylic acid (can be carboxylic acid from NSAIDs) in the presence of sodium iodide and a base (tertiary amines, Cs₂CO₃, Ag₂CO₃) to afford compound (13). Treatment of intermediate (13) with eflornithine analogs (1) or (2) in the presence of a base gives rise to eflornithine-NSAID conjugate or eflornithine prodrug with formula (14) or (15).

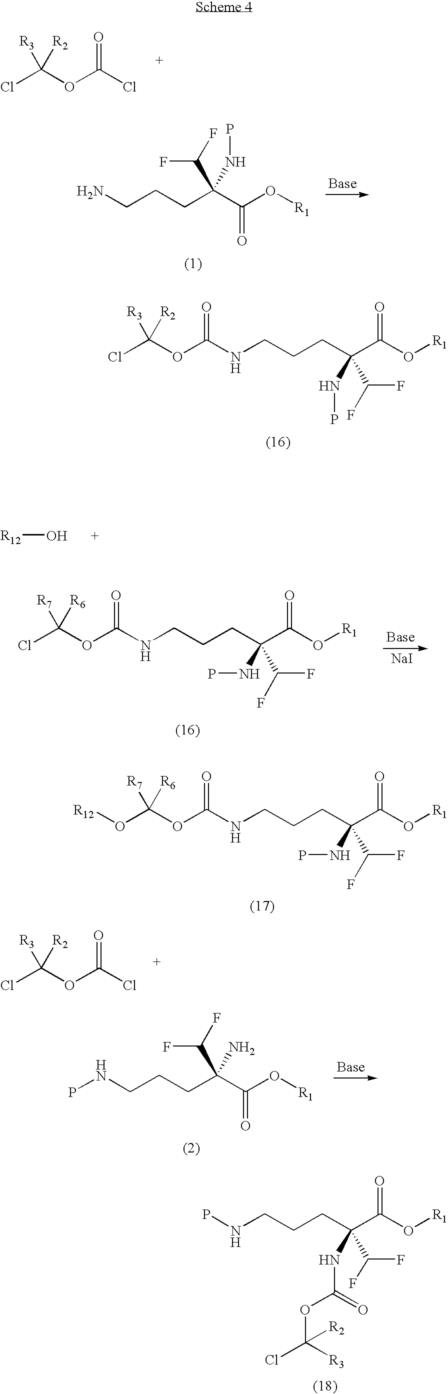

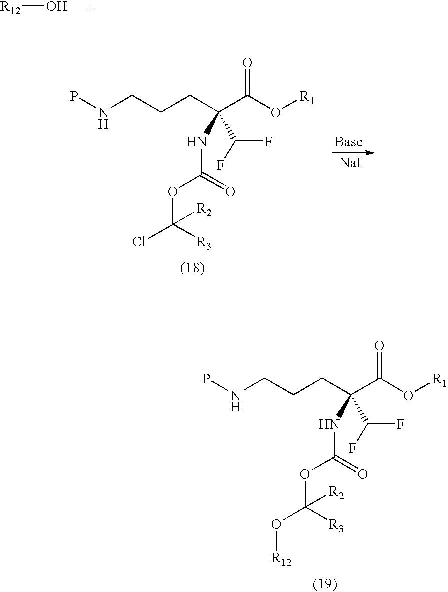

The synthesis of eflornithine-NSAIDs conjugates or eflornithine prodrug with Formula (17) or (19) is illustrated in Scheme 4. Chloroformate is first treated with eflornithine analogs (1) or (2) in the presence of a base to provide intermediate (16) or (18), which is then reacted with alcohols in particular, such as phenol moiety in the presence of sodium iodide and a base to afford final eflornithine-NSAIDs conjugate or eflornithine prodrugs (17) or (19).

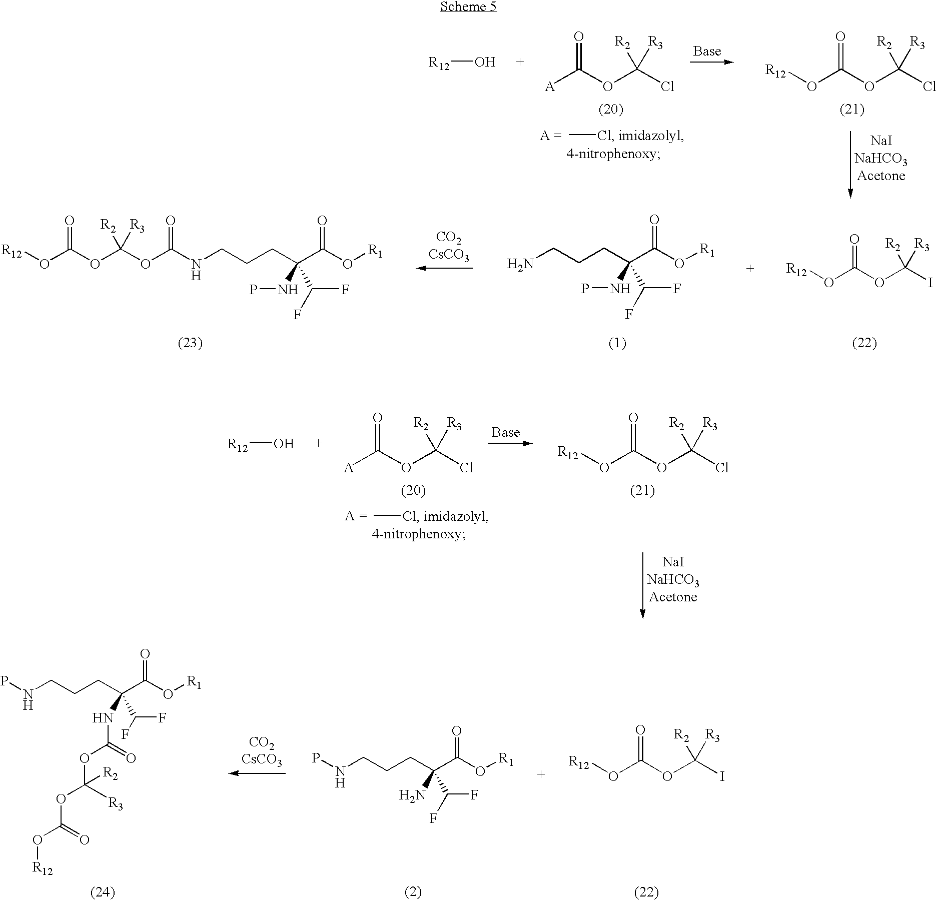

A method to synthesis of eflornithine-NSAIDs conjugate or eflornithine prodrug with Formula (23) or (24) is illustrated in Scheme 5. Alcohol is first reacted with chloroformate (or other active carbamate or carbonate) in the presence of a base. Halide interchange provides the intermediate (22), which is reacted with eflornithine analogs (1) or (2) under basic condition in the presence of carbon dioxide to afford the final desired product with formula (23) or (24).

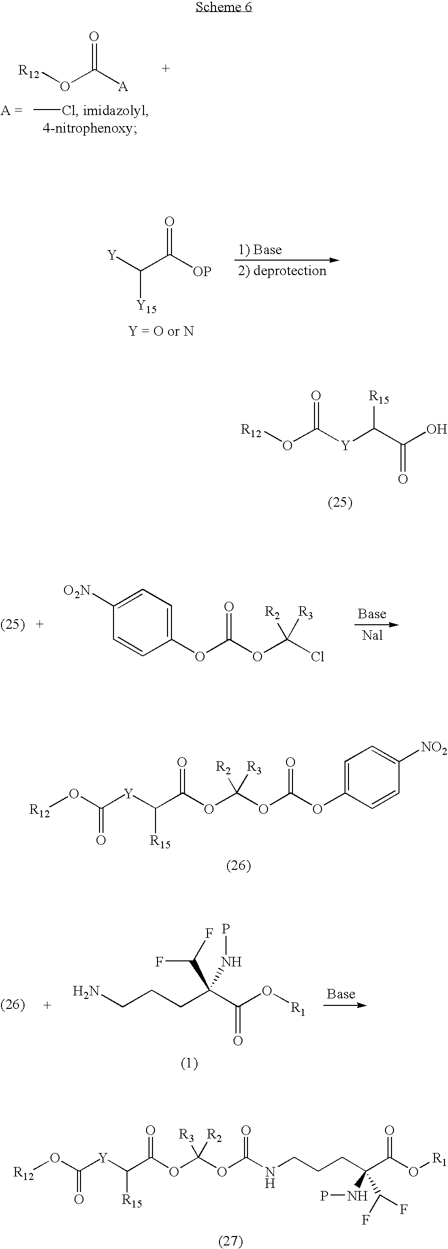

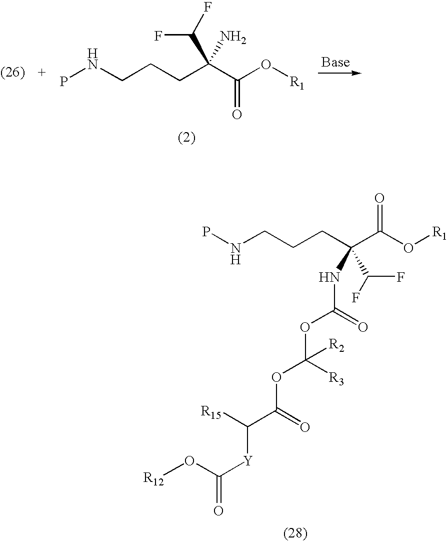

A method to synthesis of eflornithine-NSAIDs conjugate or eflornithine prodrug with Formula (26) or (28) is illustrated in Scheme 6. A chloroformate (or other active carbamate or carbonate derived from NSAIDs) is reacted with α-hydroxy alkyl acetate in the presence of a base to provide carbonate or carbamate. The ester is then deprotected to provide intermediate (25). The compound (25) is reacted with halide substituted carbonate in the presence of a base and sodium iodide to give compound (26). The compound (26) is then coupled with eflornithine analogs (1) or (2) under basic condition to afford the final compound with formula (26) or (28).

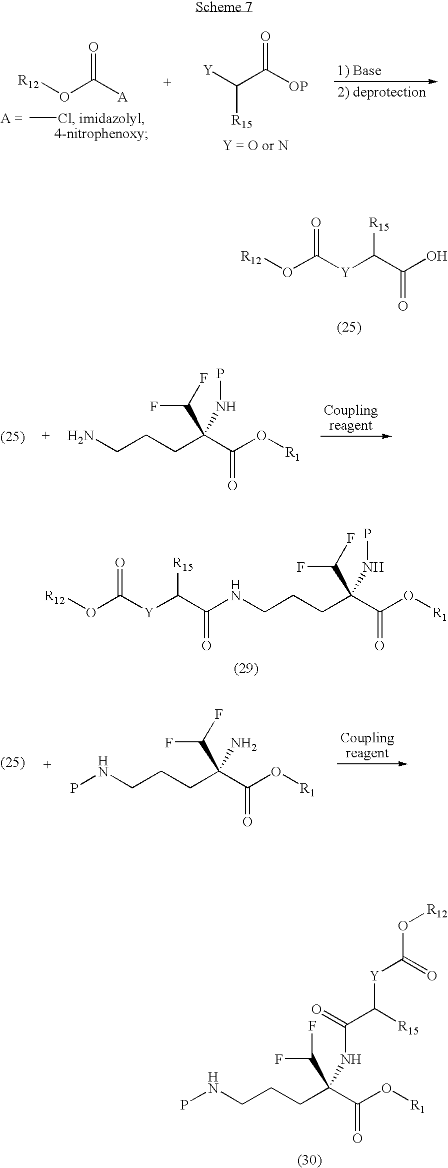

The synthesis of eflornithine-NSAIDs conjugate or eflornithine prodrug with Formula (29) or (30) is illustrated in Scheme 7. A chloroformate (or other active carbamate or carbonate) is reacted with α-hydroxy alkyl acetate under basic conditions, followed by removal of ester to provide compound (25). The compound (25) is then coupled with eflornithine analogs (1) or (2) to afford the final compound with formula (29) or (30).

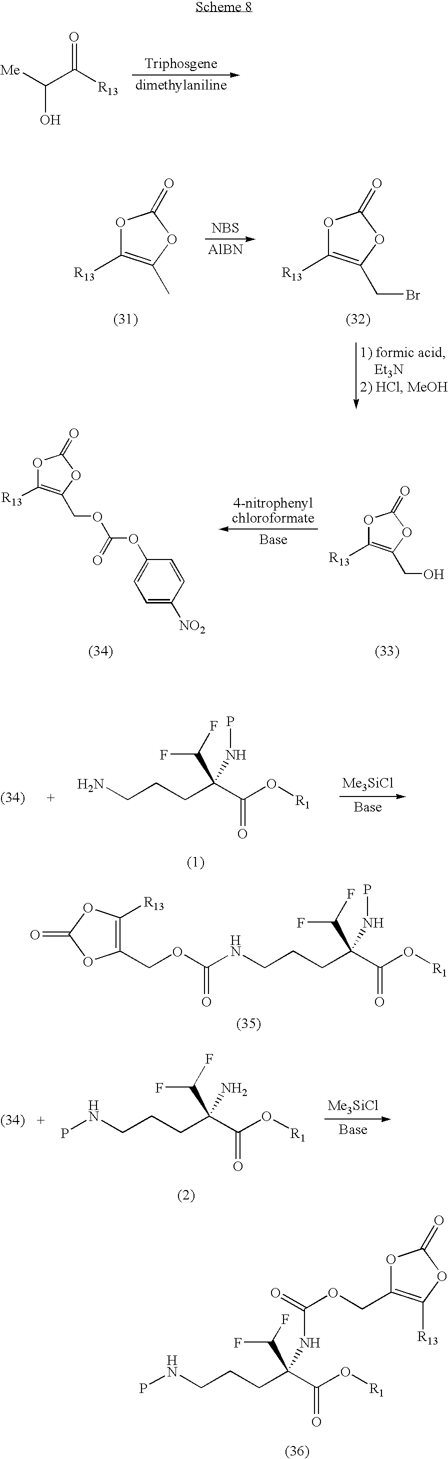

One method for synthesis of oxodioxolenylmethyl carbamate prodrug (35) or (36) is illustrated in Scheme 8. Hydroxyketone is treated with phosgene or carbonyldiimidazole in the presence of a base to yield cyclic carbonate (31). Free radical bromination with N-bromosuccinimide and azoisobutryonitrile provides bromide (32), which is converted to alcohol (33). The intermediate (33) is transformed to dicarbonate (34) by reaction with 4-nitrophenyl chloroformate, which is then reacted with eflornithine derivatives (1) or (2) to provide prodrugs (35) or (36). Alternatively, reaction of compound (33) with isocyanate (6) or (10) will also provide the desired final products.

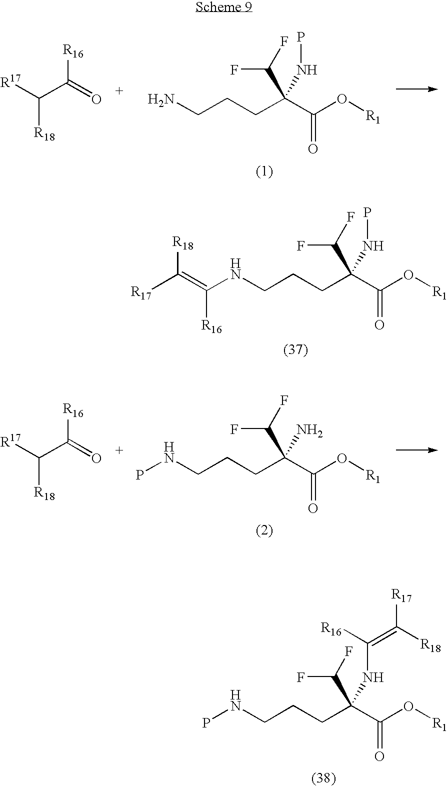

In some embodiments, the enamine prodrugs of eflornithine or eflornithine-NSAID conjugates are synthesized by reacting activated carbonyl compounds (or NSAIDs containing carbonyl functional group) with eflornithine analogs (1) or (2) as illustrated in Scheme 9. This reaction can be carried out with or without a secondary amine as catalyst under dehydrating conditions.

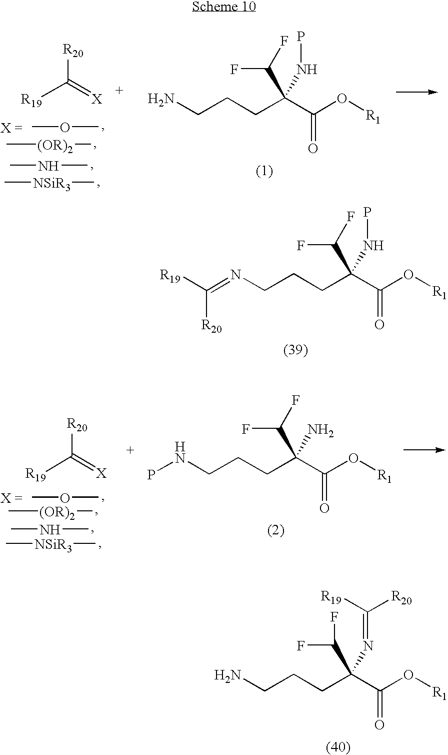

In some embodiments, imine prodrugs of eflornithine or eflornithine-NSAID conjugates are synthesized as illustrated in Scheme 10 by treating ketone or ketone equivalents with eflornithine derivatives (1) or (2) under dehydrating conditions with optional 4 A molecular sieves.

### Linkers

In the present invention, the first moiety is covalently linked to the second moiety. In some embodiments, the two moieties are linked via a linker. Linkers that can be used in this invention include but are not limited to the following:

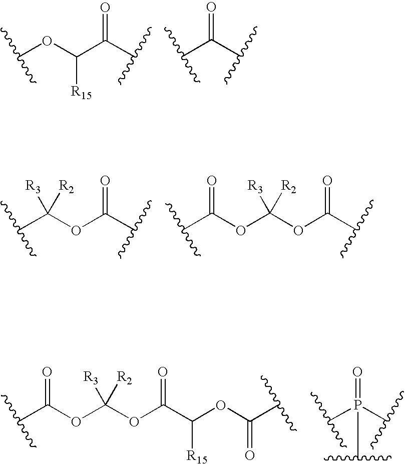

The physiologically labile linkage can be any suitable linkage that is labile under physiological conditions approximating those found in physiologic fluids, such as blood plasma. The linkage can be a direct bond (for instance, an amide, ester, carbonate, carbamate, acyloxycarbamate, sulfonate, or a sulfamate linkage) or can be a linking group (for instance a C₁-C₁₂ dialcohol, a C₁-C₁₂ hydroxylalkanoic acid, a C₁-C₁₂ hydroxyalkylamine, a C₁-C₁₂ diacid, a C₁-C₁₂ amino acid, or a C₁-C₁₂ diamine). Especially preferred linkages are direct amide, ester, carbonate, carbamate, and sulfamate linkages, and linkages via succinic acid, salicylic acid, diglycolic acid, oxa acids, oxamethylene, and halides thereof. The linkages are labile under physiologic conditions, which generally means pH of about 6 to about 8. The lability of the linkages depends upon the particular type of linkage, the precise pH and ionic strength of the physiologic fluid, and the presence or absence of enzymes that tend to catalyze hydrolysis reactions in vivo. In general, lability of the linkage in vivo is measured relative to the stability of the linkage when the compound has not been solubilized in a physiologic fluid. Thus, while some compounds according to the present invention can be relatively stable in some physiologic fluids, nonetheless, they are relatively vulnerable to hydrolysis in vivo (or in vitro, when dissolved in physiologic fluids, whether naturally occurring or simulated) as compared to when they are neat or dissolved in non-physiologic fluids (e.g. non-aqueous solvents such as acetone). Thus, the labile linkages are such that, when the drug is dissolved in an aqueous solution, especially a physiologic fluid such as blood plasma, the reaction is driven to the hydrolysis products.

While diacids, dialcohols, amino acids, and the like are described above as being suitable linkers, other linkers are encompassed within the present invention. For instance, while the hydrolysis product of a compound according to the present invention can comprise a diacid, the actual reagent used to make the linkage can be, for example, a diacylhalide, such as succinyl chloride, or an anhydride, such as succinic anhydride or diglycolic anhydride. A person having skill in the art will recognize that other possible acid, alcohol, amino, sulfato, and sulfamoyl derivatives can be used as reagents to make the corresponding linkage.

### IV. Pharmaceutical Composition of the Invention

Another aspect of the present invention relates to formulations, routes of administration and effective doses for pharmaceutical compositions comprising an eflornithine-NSAID conjugate or combination of the conjugates with other agents of the instant invention. Yet another aspect of the present invention relates to formulations, routes of administration and effective doses for pharmaceutical compositions comprising an eflornithine-aspirin salt or combination of this salt with other agents of the instant invention.

Compounds of the invention can be administered as pharmaceutical formulations including those suitable for oral (including buccal and sub-lingual), rectal, nasal, topical, transdermal patch, pulmonary, vaginal, suppository, or parenteral (including intramuscular, intraarterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippincott Williams & Wilkins, Baltimore Md. (1999). The composition is prepared in accordance with known formulation techniques. Detailed guidance for preparing compositions of the invention can be found by reference to the 18^{th} and 19^{th} Edition of Remington's Pharmaceutical. Sciences, Published by the Mack Publishing Co., Easton, Pa. 18040, which is incorporated by reference in its entirety herein.

Unit-dose or multiple-dose forms are contemplated in the invention, each offering advantages in certain clinical settings. The unit dose can contain pre-determined quantity of the active compound calculated to produce the desired effect in the setting of treating cancer. The multiple dose form can be particularly useful when multiple of single doses, or fractional doses, are required to achieve the desired outcomes. Either of these dose forms can have specifications that are dictated by or directly dependent upon the unique characteristic of the particular eflornithine-NSAID conjugate or eflornithine-aspirin salt, the particular therapeutic effect to be achieved, and any limitations inherent in the art of preparing the particular drug conjugate or salt for treatment of cancer.

In some embodiments, a unit dose contains a therapeutically effective amount sufficient to treat cancer in a subject and contains from about 1 to 1000 mg of the drug conjugate or eflornithine-aspirin salt, preferably from about 5 to 500 mg.

In some embodiments, preferred compositions of the invention are formulated for oral delivery in a suitable formulation as an ingestible tablet, a buccal tablet, capsule, caplet, elixir, suspension, syrup, trouche, wafer, lozenge, and the like. In some embodiments, the oral formulation is a tablet or a capsule. Suitable formulations are prepared in accordance to standard formulating techniques available that suit the characteristics of the compound to the excipients available for formulating an appropriate composition. In certain embodiments, a tablet or capsule contains about 5 to about 500 mg of a drug conjugate with Formula (I), (II) & (III) or eflornithine-aspirin salt of the present invention.

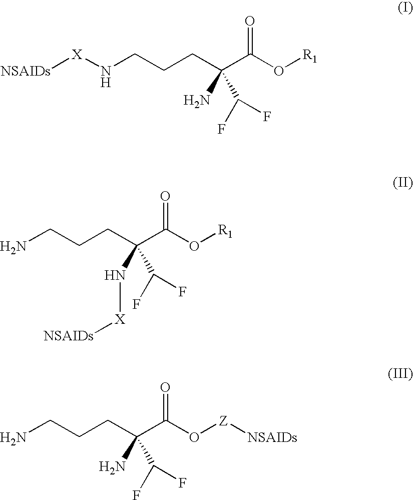

In some embodiments, the formulation is a rapid delivery of the compound or a sustained-release preparation. In some embodiments, the compound is enclosed in a hard or soft capsule, compressed into tablets, or incorporated with beverages, food or into the diet. Generally, the eflornithine-NSAID conjugates or eflornithine-aspirin salts of the invention is included at concentration levels ranging from about 0.5%, about 5%, about 10%, about 20%, or about 30% to about 50%, about 60%, about 70%, about 80% or about 90% by weight of the total composition of oral dosage forms, in an amount sufficient to provide a desired unit of dosage. Preferred compositions according to the current invention are prepared so that an oral dosage unit form contains between about 5 to about 50% by weight in dosage units weight between 5 and 1000 mg.

The suitable formulation of an oral dosage unit can also contain: a binder, such as gum tragacanth, acacia, corn starch, gelatin; a sweetening agents such as lactose or sucrose, disintegrating agents such as corn starch, alginic acid and the like; a lubricant such as magnesium stearate; or flavoring such a peppermint, oil of wintergreen. Various other materials can be present as coating or to otherwise modify the physical form of the oral dosage unit. The oral dosage unit can be coated with shellac, a sugar or both. Syrup or elixir can contain the compound, sucrose as a sweetening agent, methyl and propylparabens as preservative, a dye and flavoring. Any material utilized should be pharmaceutically acceptable and substantially non-toxic. Details of the types of excipients useful can be found in the nineteenth edition of “Remington: The Science and Practice of Pharmacy” Mack Printing Company, Easton, Pa. See particularly chapters 91-93 for a fuller discussion. Moreover, in a tablet or pill form, the compositions can be coated to delay disintegration and absorption in the gastrointestinal tract, thereby providing a sustained action over an extended period of time. Selectively permeable membranes surrounding an osmotically active driving compound are also suitable for orally administered compounds and compositions of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used.

In some embodiments, aqueous suspensions for oral use contains conjugate(s) or salts of this invention with pharmaceutically acceptable excipients, such as a suspending agent (e.g., methyl cellulose), a wetting agent (e.g., lecithin, lysolecithin and/or a long-chain fatty alcohol), as well as coloring agents, preservatives, flavoring agents, and the like. Suitable carriers, excipients or diluents include but are not limited to water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), etc. Additionally, flavoring agents, preservatives, coloring agents, bile salts, acylcarnitines and the like can be added.

Dragee cores can be provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize the active eflornithine-NSAID conjugates or eflornithine-aspirin salts or eflornithine ester-aspirin salt.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active agents can be dissolved or suspended in suitable liquids, such as fatty oils, liquid paraffin, or liquid polyethylene glycols. In addition, stabilizers can be added. All formulations for oral administration should be in dosages suitable for administration.

When formulating compounds of the invention for oral administration, it is desirable to utilize gastroretentive formulations to enhance absorption from the gastrointestinal (GI) tract. A formulation which is retained in the stomach for several hours can release compounds of the invention slowly and provide a sustained release that is preferred in some embodiments of the invention. Disclosure of such gastro-retentive formulations are found in Klausner, E. A.; Lavy, E.; Barta, M.; Cserepes, E.; Friedman, M.; Hoffman, A. 2003 “Novel gastroretentive dosage forms: evaluation of gastroretentivity and its effect on levodopa in humans.” Pharm. Res. 20, 1466-73, Hoffman, A.; Stepensky, D.; Lavy, E.; Eyal, S. Klausner, E.; Friedman, M. 2004 “Pharmacokinetic and pharmacodynamic aspects of gastroretentive dosage forms” Int. J. Pharm. 11, 141-53, Streubel, A.; Siepmann, J.; Bodmeier, R.; 2006 “Gastroretentive drug delivery systems” Expert Opin. Drug Deliver. 3, 217-3, and Chavanpatil, M. D.; Jain, P.; Chaudhari, S.; Shear, R.; Vavia, P. R. “Novel sustained release, swellable and bioadhesive gastroretentive drug delivery system for olfoxacin” Int. J. Pharm. 2006 epub March 24. In some embodiments, expandable, floating and bioadhesive techniques are utilized to maximize absorption of the compounds of the invention.

In some embodiments, the compound of the invention is administered parenterally (e.g. intravenously, intramuscularly, intravenously, subcutaneously or interperitonically). The carrier or excipient or excipient mixture can be a solvent or a dispersive medium containing, for example, various polar or nonpolar solvents, suitable mixtures thereof or oils. Examples of “carrier” or “excipient” include but are not limited to all solvents, dispersive agents or media, coatings, antimicrobial agents, iso- or hypo- or hypertonic agents, absorption modifying agents. The use of the substances is well known in the art. Moreover, other or supplementary active ingredients can also be incorporated into the final composition. In some embodiments, the pharmaceutical composition includes carriers and excipients (including but not limited to buffers, carbohydrates, mannitol, proteins, polypeptides or amino acids such as glycine, antioxidants, bacteriostats, chelating agents, suspending agents, thickening agents and/or preservatives), water, oils including but not limited to those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include, but are not limited to, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. In some embodiments, the pharmaceutical preparation is substantially free of preservatives. In other embodiments, the pharmaceutical preparation contains at least one preservative. General methodology on pharmaceutical dosage forms is found in Ansel et al., Pharmaceutical Dosage Forms and Drug Delivery Systems (Lippencott Williams & Wilkins, Baltimore Md. (1999)). It will be recognized that, while any suitable carrier known to those of ordinary skill in the art can be employed to administer the compositions of this invention, the type of carrier will vary depending on the mode of administration.

For injectable formulations, the vehicle can be chosen from those known in art to be suitable, including aqueous solutions or oil suspensions, or emulsions, with sesame oil, corn oil, cottonseed oil, or peanut oil, as well as elixirs, mannitol, dextrose, or a sterile aqueous solution, and similar pharmaceutical vehicles. In some embodiments, the formulation also comprises polymer compositions which are biocompatible, biodegradable, such as poly(lactic-co-glycolic)acid. These materials can be made into micro or nanospheres, loaded with drug and further coated or derivatized to provide superior sustained release performance Vehicles suitable for periocular or intraocular injection include, for example, suspensions of therapeutic agent in injection grade water, liposomes and vehicles suitable for lipophilic substances. Other vehicles for periocular or intraocular injection are well known in the art.

In some embodiments, the compounds of the invention are formulated as a sterile solution or suspension, in suitable vehicles, well known in the art. The pharmaceutical compositions are sterilized by conventional, well-known sterilization techniques, or sterile filtered. The resulting aqueous solutions can be packaged for use, or lyophilized, the lyophilized preparation being combined with a sterile solution prior to administration. Suitable formulations and additional carriers are described in Remington “The Science and Practice of Pharmacy” (20^{th} Ed., Lippincott Williams & Wilkins, Baltimore Md.), the teachings of which are incorporated by reference in their entirety herein. In the case of a sterile powder, the preferred methods include vacuum drying or freeze drying to which any required ingredients are added. The final pharmaceutical form must be protected from contamination. A single intravenous or intraperitoneal dose can be administrated. Alternatively, a slow long term infusion or multiple short daily infusions can be also utilized, typically lasting from 1 to 7 days. Alternate day or dosing once every day can be utilized.

In certain embodiments, the final form is sterile and is able to pass readily through an injection device such as a hollow needle. The proper viscosity can be achieved and maintained by the proper choice of solvents or excipients. The use of molecular or particulate coatings such as lecithin, the proper selection of particle size in dispersions, or the use of materials with surfactant properties can be utilized.

In a preferred embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition also includes a solubilizing agent and a local anesthetic such as lidocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients are mixed prior to administration.

When administration is by injection, the active compound can be formulated in aqueous solutions, specifically in physiologically compatible buffers such as Hanks solution, Ringer's solution, or physiological saline buffer. In some embodiments, the solution contains formulatory agents such as suspending, stabilizing and/or dispersing agents. Alternatively, the active compound can be in powder form for constitution with a suitable vehicle, e.g., sterile pyrogen-free water, before use. In some embodiments, the pharmaceutical composition does not comprise an adjuvant or any other substance added to enhance the immune response stimulated by the eflornithine-NSAID conjugate or the eflornithine-aspirin salt or eflornithine ester-aspirin salt. In some embodiments, the pharmaceutical composition comprises a substance that inhibits an immune response to the eflornithine-NSAID conjugate or eflornithine-aspirin salt or eflornithine ester-aspirin salt. Methods of formulation are known in the art, for example, as disclosed in Remington's Pharmaceutical Sciences, latest edition, Mack Publishing Co., Easton P.

In addition to the formulations described previously, the eflornithine-NSAID conjugate or eflornithine-aspirin salt or eflornithine ester-aspirin salt can also be formulated as a depot preparation. In some embodiments, such long acting formulations are administered by implantation or transcutaneous delivery (for example subcutaneously or intramuscularly), intramuscular injection or use of a transdermal patch. Thus, for example, the eflornithine-NSAID conjugate or eflornithine-aspirin salt or eflornithine ester-aspirin salt is formulated with suitable polymeric or hydrophobic materials (for example as an emulsion in an acceptable oil) or ion exchange resins, or as sparingly soluble derivatives, for example, as a sparingly soluble salt.

In some embodiments, a liposomal delivery of an eflornithine-NSAID conjugate or eflornithine-aspirin salt of the invention is provided. The system restrains the eflornithine-NSAID conjugate or eflornithine-aspirin salt or eflornithine ester-aspirin salt by incorporating, encapsulating, surrounding, or entrapping the compound in, on or by lipid vesicles or liposomes, or by micelles. The liposomes include but are not limited to, lipids such as cholesterol, phospholipids, or micelles composed of surfactants such as sodium dodecylsylfate, octylphenolpolyoxyethylene glycol, or sorbitan monooleate.

The concentration of drug can be adjusted, the pH of the solution buffered and the isotonicity adjusted to be compatible with intravenous injection, as is well known in the art.

The compounds or their pharmaceutically acceptable salts can be provided alone or in combination with one or more other agents or with one or more other forms. For example a formulation comprises one or more agents in particular proportions, depending on the relative potencies of each agent and the intended indication. For example, in compositions for targeting two different host targets, and where potencies are similar, about a 1:1 ratio of agents can be used. The two forms can be formulated together, in the same dosage unit e.g., in one cream, suppository, tablet, capsule, aerosol spray, or packet of powder to be dissolved in a beverage; or each form can be formulated in a separate unit, e.g., two creams, two suppositories, two tablets, two capsules, a tablet and a liquid for dissolving the tablet, two aerosol sprays, or a packet of powder and a liquid for dissolving the powder, etc.

Typical pharmaceutically acceptable salts are those of the inorganic ions, such as, for example, sodium, potassium, calcium, magnesium ions, and the like. Such salts include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, if the agent(s) contain a carboxy group or other acidic group, it can be converted into a pharmaceutically acceptable addition salt with inorganic or organic bases. Examples of suitable bases include, but are not limited to, sodium hydroxide, potassium hydroxide, ammonia, cyclohexylamine, dicyclohexyl-amine, ethanolamine, diethanolamine, triethanolamine, and the like.

A pharmaceutically acceptable ester or amide includes, but is not limited to, ethyl, methyl, isobutyl, ethylene glycol, and the like. Typical amides include, but are not limited to, unsubstituted amides, alkyl amides, dialkyl amides, and the like.

In some embodiments, pharmaceutical compositions comprising an eflornithine-NSAID conjugate or eflornithine-aspirin salt or eflornithine ester-aspirin salt of the present invention exert local and regional effects when administered topically or injected at or near particular sites of pain. In some embodiments, direct topical application, e.g., of a viscous liquid, solution, suspension, dimethylsulfoxide (DMSO)-based solutions, liposomal formulations, gel, jelly, cream, lotion, ointment, suppository, foam, or aerosol spray, is used for local administration, to produce for example local and/or regional effects. Pharmaceutically appropriate vehicles for such formulation include, for example, lower aliphatic alcohols, polyglycols (e.g., glycerol or polyethylene glycol), esters of fatty acids, oils, fats, silicones, and the like. Such preparations also include preservatives (e.g., p-hydroxybenzoic acid esters) and/or antioxidants (e.g., ascorbic acid and tocopherol). See also Dermatological Formulations: Percutaneous absorption, Barry (Ed.), Marcel Dekker Incl, 1983.

The compositions according to the present invention can be in any form suitable for topical application, including but not limited to aqueous, aqueous-alcoholic or oily solutions, lotion or serum dispersions, aqueous, anhydrous or oily gels, emulsions obtained by dispersion of a fatty phase in an aqueous phase (O/W or oil in water) or, conversely, (W/O or water in oil), microemulsions or alternatively microcapsules, microparticles or lipid vesicle dispersions of ionic and/or nonionic type. These compositions can be prepared according to conventional methods. In formulating skin ointments, an eflornithine-NSAID conjugate or eflornithine-aspirin salt or eflornithine ester-aspirin salt of the instant invention can be formulated in an oleaginous hydrocarbon base, an anhydrous absorption base, a water-in-oil absorption base, an oil-in-water water-removable base and/or a water-soluble base. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols. The construction and use of transdermal patches for the delivery of pharmaceutical agents is well known in the art. See, e.g., U.S. Pat. Nos. 5,023,252, 4,992,445 and 5,001,139.

In some embodiments, cancers associated with the respiratory system are effectively treated with aerosol solutions, suspensions or dry powders comprising an eflornithine-NSAID conjugate or eflornithine-aspirin salt or eflornithine ester-aspirin salt of the present invention. The aerosol can be administered through the respiratory system or nasal passages. For example, one skilled in the art will recognize that a composition of the present invention can be suspended or dissolved in an appropriate carrier, e.g., a pharmaceutically acceptable propellant, and administered directly into the lungs using a nasal spray or inhalant. An aerosol formulation for nasal administration is generally an aqueous solution designed to be administered to the nasal passages in drops or sprays. Nasal solutions can be similar to nasal secretions in that they are generally isotonic and slightly buffered to maintain a pH of about 5.5 to about 6.5, although pH values outside of this range can additionally be used. An aerosol formulation for inhalations and inhalants can be designed so that the eflornithine-NSAID conjugate or eflornithine-aspirin salt or eflornithine ester-aspirin salt of the present invention is carried into the respiratory tree of the subject when administered by the nasal or oral respiratory route.

In some embodiments, cancers associated with eye can be effectively treated with ophthalmic solutions, suspensions, ointments or inserts comprising an eflornithine-NSAID conjugate or eflornithine-aspirin salt or eflornithine ester-aspirin salt of the present invention. In some embodiments, cancers associated with the ear can be effectively treated with otic solutions, suspensions, ointments or inserts comprising an eflornithine-NSAID conjugate or eflornithine-aspirin salt or eflornithine ester-aspirin salt of the present invention. In some embodiments, the compounds of the invention are formulated for vaginal administration. In some embodiments, gastrointestinal cancers are effectively treated with orally- or rectally delivered solutions, suspensions, ointments, enemas and/or suppositories comprising an eflornithine-NSAID conjugate or eflornithine-aspirin salt or eflornithine ester-aspirin salt of the present invention.

It is envisioned additionally, that the compounds of the invention can be attached releasably to biocompatible polymers for use in sustained release formulations on, in or attached to inserts for topical, intraocular, periocular, or systemic administration. The controlled release from a biocompatible polymer can be utilized with a water soluble polymer to form an instillable formulation, as well. The controlled release from a biocompatible polymer, such as for example, PLGA microspheres or nanospheres, can be utilized in a formulation suitable for intra ocular implantation or injection for sustained release administration as well. Any suitable biodegradable and biocompatible polymer can be used.

When an eflornithine-NSAID conjugate of the invention is acidic, it can be included in any of the above-described formulations as the free acid, a pharmaceutically acceptable salt, a solvate or hydrate. In some embodiments, pharmaceutically acceptable salts that substantially retain the activity of the free acid are prepared by reaction with bases and tend to be more soluble in aqueous and other protic solvents than the corresponding free acid form.

Pharmaceutical compositions comprising an eflornithine-NSAID conjugate of the invention can be manufactured by means of conventional mixing, dissolving, granulating, dragee-making, levigating, emulsifying, encapsuling, entrapping or lyophilizing process.

### V. Method of Treatment

In one aspect, the current invention provides prophylactic and therapeutic cancer treatment methods by administration to a subject in need thereof a therapeutically effective amount of a compound or a pharmaceutical composition of the invention. In some embodiments, the compound of the invention comprises a first moiety and a second moiety, the first moiety being covalently linked to the second moiety, wherein the first moiety is an eflornithine or an analog or derivative of eflornithine, and the second moiety is an NSAID.

In another aspect, the current invention provides a method of treating cancer by administration of a compound of formula (IV) or (V), i.e. an eflornithine prodrug. In a preferred embodiment, the eflornithine prodrug is administered to a subject, preferably a human, via oral administration. In some embodiments, the eflornithine prodrug is administered alone. In other embodiments, the eflornithine prodrug is administered in combination with an NSAID disclosed herein. In some embodiments, the NSAID is administered prior to, concomitant with, or subsequent to administering the compound of formula (IV) or (V).

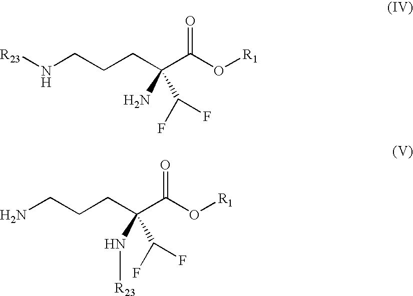

A suitable subject can be, e.g., a human, a non-human primate (including but not limited to a gorilla, chimpanzee, orangutan, or a monkey), a rodent (including but not limited to a mouse, rat, guinea pig, or gerbil) a dog, a cat, horse, cow, pig, sheep, rabbit, or goat. The subject is preferably a mammal, and most preferably a human.

In some embodiments, an eflornithine-NSAID conjugate or eflornithine-aspirin salt or eflornithine ester-aspirin salt and/or a pharmaceutical composition of the invention is administered to a mammal, preferably a human, to treat a cancer including but not limited to acute lymphoblastic leukemia, adult acute lymphoblastic leukemia, childhood acute myeloid leukemia, adult acute myeloid leukemia, adrenocortical carcinoma, childhood adrenocortical carcinoma, AIDS-related cancers, AIDS-related lymphoma, anal cancer, appendix cancer, childhood cerebellar astrocytoma, childhood cerebral astrocytoma, basal cell carcinoma, extrahepatic bladder cancer, child hood bladder cancer, osteosarcoma i.e. bone cancer, malignant fibrous histiocytoma, childhood brain stem glioma, brain tumor—cerebellar astrocytoma, brain Tumor—cerebral astrocytoma/malignant glioma—childhood; brain tumor—ependymoma, brain tumor—medulloblastoma, brain tumor—supratentorial primitive neuroectodermal tumors, brain tumor—visual pathway and hypothalamic glioma, brain tumor—other, breast cancer, bronchial adenoma. carcinoids, Burkitt lymphoma, carcinoid tumor—childhood, carcinoid tumor—gastrointestinal, carcinoma of unknown primary, central nervous system lymphoma-primary, cervical cancer, childhood cancers, chronic lymphocytic leukemia; chronic myelogenous leukemia, chronic myeloproliferative disorders, colon cancer, colorectal cancer, cutaneous T-cell lymphoma; desmoplastic small round cell tumor, endometrial cancer, ependymoma, esophageal cancer, Ewing's sarcoma (in the Ewing family of tumors), extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor—extracranial, germ cell tumor—extragonadal, germ cell tumor—ovarian, gestational trophoblastic tumor, adult glioma, childhood brain stem glioma, childhood cerebral astrocytoma glioma, childhood visual pathway and hypothalamic glioma, gastric carcinoid; hairy cell leukemia, head and neck cancer, hepatocellular (liver) cancer, adult (Primary), hepatocellular (liver) cancer-childhood (primary), Hodgkin lymphoma-adult and childhood, Hodgkin lymphoma during pregnancy, hypopharyngeal cancer, hypothalamic and visual pathway glioma-childhood, intraocular melanoma, islet cell carcinoma (endocrine pancreas), Kaposi's sarcoma, kidney (renal cell) cancer, childhood kidney cancer, laryngeal cancer, Leukemia, acute lymphoblastic, adult; Leukemia, acute lymphoblastic, childhood; Leukemia, acute myeloid, adult; Leukemia, acute myeloid, childhood; Leukemia, chronic lymphocytic; Leukemia, chronic myelogenous; Leukemia, hairy cell; lip and oral cavity cancer; liver cancer, adult (primary); liver cancer-childhood (primary); lung cancer, non-small cell; lung cancer—small cell; lymphoma, AIDS-related; Lymphoma, Burkitt; Lymphoma, cutaneous T-Cell; lymphoma, Hodgkin, adult; Lymphoma, Hodgkin, childhood; Lymphoma, non-Hodgkin, adult; Lymphoma, Non-hodgkin, childhood; Lymphoma, non-Hodgkin during pregnancy; Lymphoma, Primary Central Nervous System; macroglobulinemia-Waldenstrom, malignant fibrous histiocytoma of Bone/osteosarcoma; childhood medulloblastoma, melanoma, melanoma-intraocular (eye), Merkel cell carcinoma, adult malignant mesothelioma, childhood mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma (cancer of the bone-marrow), chronic myeloproliferative disorders, nasal cavity and paranasal sinus cancer, nasopharyngeal carcinoma, childhood nasopharyngeal cancer, neuroblastoma, non-Hodgkin lymphoma, adult; non-Hodgkin lymphoma, childhood; non-Hodgkin lymphoma during pregnancy, non-small cell lung cancer, oral cancer-childhood, oral cavity cancer-lip and oropharyngeal cancer; osteosarcoma/malignant fibrous histiocytoma of bone, childhood ovarian cancer, ovarian epithelial cancer, ovarian germ cell tumor, ovarian low malignant potential tumor, pancreatic cancer, childhood pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary tumor, plasma cell neoplasm/multiple myeloma, pleuropulmonary blastoma, pregnancy and breast cancer, pregnancy and Hodgkin lymphoma, pregnancy and Non-Hodgkin lymphoma, primary central nervous system lymphoma, prostate cancer, rectal cancer, renal cell (kidney) cancer, childhood renal Cell (kidney) cancer, renal pelvis and ureter-transitional cell cancer, retinoblastoma, childhood rhabdomyosarcoma, salivary gland cancer, childhood salivary gland cancer, sarcoma—Ewing family of tumors, Kaposi sarcoma, adult soft tissue sarcoma, childhood soft tissue sarcoma, uterine sarcoma, Sézary syndrome, skin cancer (nonmelanoma), childhood skin cancer, skin cancer (melanoma), Merkel cell skin carcinoma, small cell lung cancer, small intestine cancer, squamous cell carcinoma (nonmelanoma), metastatic squamous neck cancer with occult primary, stomach (gastric) cancer, childhood stomach (gastric) cancer, childhood supratentorial primitive neuroectodermal tumors, cutaneous T-cell lymphoma, testicular cancer, throat cancer, childhood thymoma, thymoma and thymic carcinoma, thyroid cancer, childhood thyroid cancer, transitional cell cancer of the renal pelvis and ureter, gestational trophoblastic tumor, unknown primary site carcinoma of adult, unknown primary site cancer of childhood, urethral cancer, endometrial uterine cancer, uterine sarcoma, vaginal cancer, childhood visual Pathway and hypothalamic glioma, vulvar cancer, Waldenstrom macroglobulinemia, and Wilms tumor.

In some embodiments, the cancers also include cancers arising from the following: fibrosarcoma, liposarcoma, chondrosarcoma, osteogenic sarcoma, angiosarcoma, lymphangiosarcoma, synovial sarcoma, mesothelioma, invasive meningioma, leukemias, malignant lymphomas, leiomysarcoma, rhabdomyosarcoma, squamous cell or epidermoid carcinoma, basal cell carcinoma, adenocarcinoma, papillary carcinoma, cystadenocarcinoma, bronchogenic carcinoma, bronchial adenoma, malignant melanoma, renal cell carcinoma, hepatocellular carcinoma, transitional cell carcinoma, choriocarinoma, seminoma, embryonal carcinoma, malignant mixed tumor of salivary gland origin, malignant cystosarcoma, phyllodes, Wilms tumor, immature teratoma, and teratocarcinoma.

In some embodiments, the cancer is a cancer formed at a different site of a body as a result of migration of a cell from a cancer (i.e. metastasis) including but not limited to any cancer mentioned herein.

In other embodiments, the eflornithine-NSAID conjugates or eflornithine-aspirin salts or eflornithine ester-aspirin salt and/or compositions of the invention are used for the prevention of one cancer or metastasis of one cancer and concurrently for the treatment of another cancer mentioned hereinabove.

The present invention also involves the delivery of therapeutic compounds to subjects exhibiting pre-cancerous symptoms to prevent the onset of cancer. Cells of this category include but are not limited to polyps and other precancerous lesions, premalignancies, preneoplastic or other aberrant phenotype indicating probable progression to a cancerous state.

### Examples of Cancer and Hyperproliferative Disorder

### Kirsten-Ras Dependent Cancers

Ras defines a protooncogene product that is found on chromosome 11. It is found in normal cells, where it helps to relay signals by acting as a switch (Lowy and Willumsen, 1993). When receptors on the cell surface are stimulated (by a hormone, for example), Ras is switched on and transduces signals that tell the cell to grow. If the cell-surface receptor is not stimulated, Ras is not activated and so the pathway that results in cell growth is not initiated. In about 30% of human cancers, Ras is mutated so that it is permanently switched on, telling the cell to grow regardless of whether receptors on the cell surface are activated or not. Point mutations in the cellular ras gene (c-ras) also can result in a mutant p21 protein that can transform mammalian cells.

Ras is a family of retrovirus-associated DNA sequences originally isolated from Harvey (H-ras, Ha-ras, rasH) and Kirsten (K-ras, Ki-ras, rasK) murine sarcoma viruses. Ras genes are widely conserved among animal species and sequences corresponding to both H-ras and K-ras genes have been detected in human, avian, murine, and non-vertebrate genomes. The closely related N-ras gene has been detected in human neuroblastoma and sarcoma cell lines. All genes of the family have a similar exon-intron structure and each encodes a p21 protein.

### Breast Cancer

In some embodiments, the invention provides a method of treating breast cancer comprising administering an effective amount of an eflornithine-NSAID conjugate or eflornithine-aspirin salt or a composition thereof.

Several types of breast cancer exist that can be treated by the methods provided by the invention. A lobular carcinoma in situ and a ductal carcinoma in situ are breast cancers that have developed in the lobules and ducts, respectively, but have not spread to the fatty tissue surrounding the breast or to other areas of the body. An infiltrating (or invasive) lobular and a ductal carcinoma are cancers that have developed in the lobules and ducts, respectively, and have spread to either the breast's fatty tissue and/or other parts of the body. Other cancers of the breast that would benefit from treatment by the methods provided by the invention are medullary carcinomas, colloid carcinomas, tubular carcinomas, and inflammatory breast cancer.

Breast cancer is generally treated with a combination of surgery to remove the cancerous lesion and adjuvant therapy—radiation, chemotherapy or both—to attack any cancer cells that can be left after the surgery. Breast cancer can be classified broadly by the presence or absence of hormone receptors (HRs). Hormone receptor positive (HR+) cancer is characterized by the expression of one or both female hormone receptors estrogen receptor (ER) or progesterone receptor (PR). Adjuvant therapy for ER+ breast cancer often includes chemotherapy with a selective estrogen receptor modulator (SERM), such as tamoxifen or raloxifene. Unfortunately, while about 70% of breast cancers are ER positive, the remaining 30% of breast cancers that are HR negative are not amenable to treatment with SERMs. Accordingly, other adjuvant chemotherapies, such as treatment with an anthracycline (alone or in combination with a taxane) have been tried on ER negative breast cancer. In particular, so-called triple negative metastatic breast cancer (i.e. breast cancer that is ER negative, PR negative and human epidermal growth factor receptor 2 (HER2) negative) is refractory to standard treatments and is entirely refractory to SERM chemotherapy.

Chemotherapy utilizes anti-tumor agents to prevent cancer cells from multiplying, invading, metastasizing and killing a patient. Several drugs are available to treat breast cancer, including cytotoxic drugs such as doxorubicin, cyclophosphamide, methotrexate, paclitaxel, thiotepa, mitoxantrone, vincristine, or combinations thereof. Endocrine therapy can be an effective treatment where the remaining breast tissue retains endocrine sensitivity. Agents administered for this therapy include tamoxifen, megestrol acetate, aminoglutethimide, fluoxymesterone, leuprolide, goserelin, and prednisone.

The methods provided by the invention can provide a beneficial effect for breast cancer patients, by administration of an eflornithine-NSAID conjugate or eflornithine-aspirin salt or eflornithine ester-aspirin salt or a composition thereof, and surgery, radiation therapy, chemotherapy, or endocrine therapy.

### Ovarian Cancer

In some embodiments, the invention provides a method of treating ovarian cancer, including epithelial ovarian tumors. In some embodiments, the method comprises administering an eflornithine-NSAID conjugate or eflornithine-aspirin salt or a composition thereof into a subject. Preferably, the invention provides a method of treating an ovarian cancer selected from the following: an adenocarcinoma in the ovary and an adenocarcinoma that has migrated from the ovary into the abdominal cavity. Surgery, immunotherapy, chemotherapy, hormone therapy, radiation therapy, or a combination thereof, are some possible treatments available for ovarian cancer. Some possible surgical procedures include debulking, and a unilateral or bilateral oophorectomy and/or a unilateral or bilateral salpigectomy.

Anti-cancer drugs that can be used include cyclophosphamide, etoposide, altretamine, and ifosfamide. Hormone therapy with the drug tamoxifen can be used to shrink ovarian tumors. Radiation therapy can be external beam radiation therapy and/or brachytherapy.

The methods provided by the invention can provide a beneficial effect for ovarian cancer patients, by administration of an eflornithine-NSAID conjugate or eflornithine-aspirin salt or eflornithine ester-aspirin salt or a composition thereof, and surgery, radiation therapy, chemotherapy endocrine therapy, or a combination thereof.

### Cervical Cancer

In some embodiments, the invention provides a method of treating cervical cancer, preferably an adenocarcinoma in the cervix epithelial. In some embodiments, the method comprises administering an eflornithine-NSAID conjugate or eflornithine-aspirin salt or a composition thereof into a subject.

Two main types of this cancer exist: squamous cell carcinoma and adenocarcinomas. The former constitutes about 80-90% of all cervical cancers and develops where the ectocervix (portion closest to the vagina) and the endocervix (portion closest to the uterus) join. The latter develop in the mucous-producing gland cells of the endocervix. Some cervical cancers have characteristics of both of these and are called adenosquamous carcinomas or mixed carcinomas.

The chief treatments available for cervical cancer are surgery, immunotherapy, radiation therapy and chemotherapy. Some possible surgical options are cryosurgery, a hysterectomy, and a radical hysterectomy. Radiation therapy for cervical cancer patients includes external beam radiation therapy or brachytherapy. Anti-cancer drugs that can be administered as part of chemotherapy to treat cervical cancer include cisplatin, carboplatin, hydroxyurea, irinotecan, bleomycin, vincrinstine, mitomycin, ifosfamide, fluorouracil, etoposide, methotrexate, and combinations thereof.

The methods provided by the invention can provide a beneficial effect for cervical cancer patients, by administration of an eflornithine-NSAID conjugate or eflornithine-aspirin salt or eflornithine ester-aspirin salt or a composition thereof, and surgery, radiation therapy, chemotherapy, or a combination thereof.

### Prostate Cancer

In some embodiments, the invention provides methods to treat prostate cancer, preferably a prostate cancer selected from the following: an adenocarcinoma or an adenocarinoma that has migrated to the bone. In some embodiments, the method comprises administering an eflornithine-NSAID conjugate or eflornithine-aspirin salt or a composition thereof into a subject.

Prostate cancer develops in the prostate organ in men, which surrounds the first part of the urethra. The prostate has several cell types but 99% of tumors are adenocarcinomas that develop in the glandular cells responsible for generating seminal fluid.

Surgery, immunotherapy, radiation therapy, cryosurgery, hormone therapy, and chemotherapy are some treatments available for prostate cancer patients. Possible surgical procedures to treat prostate cancer include radical retropubic prostatectomy, a radical perineal prostatectomy, and a laparscopic radical prostatectomy. Some radiation therapy options are external beam radiation, including three dimensional conformal radiation therapy, intensity modulated radiation therapy, and conformal proton beam radiation therapy. Brachytherapy (seed implantation or interstitial radiation therapy) is also an available method of treatment for prostate cancer. Cryosurgery is another possible method used to treat localized prostate cancer cells.

Hormone therapy, also called androgen deprivation therapy or androgen suppression therapy, can be used to treat prostate cancer. Several methods of this therapy are available including an orchiectomy in which the testicles, where 90% of androgens are produced, are removed. Another method is the administration of luteinizing hormone-releasing hormone (LHRH) analogs to lower androgen levels. The LHRH analogs available include leuprolide, goserelin, triptorelin, and histrelin. An LHRH antagonist can also be administered, such as abarelix.

Treatment with an antiandrogen agent, which blocks androgen activity in the body, is another available therapy. Such agents include flutamide, bicalutamide, and nilutamide. This therapy is typically combined with LHRH analog administration or an orchiectomy, which is termed a combined androgen blockade (CAB).

Chemotherapy can be appropriate where a prostate tumor has spread outside the prostate gland and hormone treatment is not effective. Anti-cancer drugs such as doxorubicin, estramustine, etoposide, mitoxantrone, vinblastine, paclitaxel, docetaxel, carboplatin, and prednisone can be administered to slow the growth of prostate cancer, reduce symptoms and improve the quality of life.

The methods provided by the invention can provide a beneficial effect for prostate cancer patients, by administration of an eflornithine-NSAID conjugate or eflornithine-aspirin salt or eflornithine ester-aspirin salt or a composition thereof, and surgery, radiation therapy, chemotherapy, hormone therapy, or a combination thereof.

### Pancreatic Cancer

In some embodiments, the invention provides methods of treating pancreatic cancer, preferably a pancreatic cancer selected from the following: an epitheliod carcinoma in the pancreatic duct tissue and an adenocarcinoma in a pancreatic duct. In some embodiments, the method comprises administering an eflornithine-NSAID conjugate or eflornithine-aspirin salt or a composition thereof into a subject.

Pancreatic cancer is the fourth-leading cause of cancer mortality among adults in the United States. One of the most promising drugs in pancreatic cancer therapy is oxaliplatin, an organoplatinum molecule, that forms inter- and intrastrand DNA adducts/cross-links and induces a high proportion of DNA single strand breaks. However, the gemcitabine and oxaliplatin combination has failed to demonstrate a statistically significant advantage compared with single-agent gemcitabine. Development of agents and drug combinations are urgently needed.

The most common type of pancreatic cancer is an adenocarcinoma, which occurs in the lining of the pancreatic duct. The possible treatments available for pancreatic cancer are surgery, immunotherapy, radiation therapy, and chemotherapy. Possible surgical treatment options include a distal or total pancreatectomy and a pancreaticoduodenectomy (Whipple procedure).

Radiation therapy can be an option for pancreatic cancer patients, specifically external beam radiation where radiation is focused on the tumor by a machine outside the body. Another option is intraoperative electron beam radiation administered during an operation.

Chemotherapy can be used to treat pancreatic cancer patients. Appropriate anti-cancer drugs include 5-fluorouracil (5-FU), mitomycin, ifosfamide, doxorubicin, steptozocin, chlorozotocin, and combinations thereof.

The methods provided by the invention can provide a beneficial effect for pancreatic cancer patients, by administration of an eflornithine-NSAID conjugate or eflornithine-aspirin salt or eflornithine ester-aspirin salt or a composition thereof, and surgery, radiation therapy, or chemotherapy.

### Bladder Cancer

In some embodiments, the invention provides methods of treating bladder cancer, preferably a transitional cell carcinoma in urinary bladder. In some embodiments, the method comprises administering an eflornithine-NSAID conjugate or eflornithine-aspirin salt or a composition thereof into a subject.

Bladder cancers are urothelial carcinomas (transitional cell carcinomas) or tumors in the urothelial cells that line the bladder. The remaining cases of bladder cancer are squamous cell carcinomas, adenocarcinomas, and small cell cancers. Several subtypes of urothelial carcinomas exist depending on whether they are noninvasive or invasive and whether they are papillary, or flat. Noninvasive tumors are in the urothelium, the innermost layer of the bladder, while invasive tumors have spread from the urothelium to deeper layers of the bladder's main muscle wall. Invasive papillary urothelial carcinomas are slender finger-like projections that branch into the hollow center of the bladder and also grow outward into the bladder wall. Non-invasive papillary urothelial tumors grow towards the center of the bladder. While a non-invasive, flat urothelial tumor (also called a flat carcinoma in situ) is confined to the layer of cells closest to the inside hollow part of the bladder, an invasive flat urothelial carcinoma invades the deeper layer of the bladder, particularly the muscle layer.

To treat bladder cancer, surgery, radiation therapy, immunotherapy, chemotherapy, or a combination thereof can be applied. Some possible surgical options are a transurethral resection, a cystectomy, or a radical cystectomy. In some embodiments, radiation therapy for bladder cancer includes but is not limited to external beam radiation and brachytherapy.

Immunotherapy is another method that can be used to treat a bladder cancer patient. Typically this is accomplished intravesically, which is the administration of a treatment agent directly into the bladder by way of a catheter. One method is Bacillus Calmete-Guerin (BCG) where a bacterium sometimes used in tuberculosis vaccination is given directly to the bladder through a catheter. The body mounts an immune response to the bacterium, thereby attacking and killing the cancer cells.

Another method of immunotherapy is the administration of interferons, glycoproteins that modulate the immune response. Interferon alpha is often used to treat bladder cancer.

Anti-cancer drugs that can be used in chemotherapy to treat bladder cancer include thitepa, methotrexate, vinblastine, doxorubicin, cyclophosphamide, paclitaxel, carboplatin, cisplatin, ifosfamide, gemcitabine, or combinations thereof.

The methods provided by the invention can provide a beneficial effect for bladder cancer patients, by administration of an eflornithine-NSAID conjugate or eflornithine-aspirin salt or eflornithine ester-aspirin salt or a composition thereof, and surgery, radiation therapy, immunotherapy, chemotherapy, or a combination thereof.

### B-Cell Lymphomas

Non-Hodgkin's Lymphomas caused by malignant (cancerous) B-Cell lymphocytes represent a large subset (about 85% in the US) of the known types of lymphoma (the other 2 subsets being T-Cell lymphomas and lymphomas where the cell type is the Natural Killer Cell or unknown). Cells undergo many changes in their life cycle dependent on complex signaling processes between cells and interaction with foreign substances in the body. Various types of lymphoma or leukemia can occur in the B-Cell life cycle.

### Acute Myeloid Leukemia

In some embodiments, the invention provides methods of treating acute myeloid leukemia (AML), preferably acute promyelocytic leukemia in peripheral blood. In some embodiments, the method comprises administering an eflornithine-NSAID conjugate or eflornithine-aspirin salt or a composition thereof into a subject.

AML begins in the bone marrow but can spread to other parts of the body including the lymph nodes, liver, spleen, central nervous system, and testes. It is acute meaning it develops quickly and can be fatal if not treated within a few months. AML is characterized by immature bone marrow cells usually granulocytes or monocytes, which continue to reproduce and accumulate.

AML can be treated by immunotherapy, radiation therapy, chemotherapy, bone marrow or peripheral blood stem cell transplantation, or a combination thereof. Radiation therapy includes external beam radiation and can have side effects. Anti-cancer drugs that can be used in chemotherapy to treat AML include cytarabine, anthracycline, anthracenedione, idarubicin, daunorubicin, idarubicin, mitoxantrone, thioguanine, vincristine, prednisone, etoposide, or a combination thereof.

Monoclonal antibody therapy can be used to treat AML patients. Small molecules or radioactive chemicals can be attached to these antibodies before administration to a patient in order to provide a means of killing leukemia cells in the body. The monoclonal antibody, gemtuzumab ozogamicin, which binds CD33 on AML cells, can be used to treat AML patients unable to tolerate prior chemotherapy regimens. Bone marrow or peripheral blood stem cell transplantation can be used to treat AML patients. Some possible transplantation procedures are an allogenic or an autologous transplant.

The methods provided by the invention can provide a beneficial effect for leukemia patients, by administration of an eflornithine-NSAID conjugate or eflornithine-aspirin salt or a composition thereof, and surgery, radiation therapy, chemotherapy, or transplantation therapy.

There are other types of leukemia's that can also be treated by the methods provided by the invention including but not limited to, Acute Lymphocytic Leukemia, Acute Myeloid Leukemia, Chronic Lymphocytic Leukemia, Chronic Myeloid Leukemia, Hairy Cell Leukemia, Myelodysplasia, and Myeloproliferative Disorders.

### Lung Cancer

In some embodiments, the invention provides methods to treat lung cancer. In some embodiments, the method comprises administering an eflornithine-NSAID conjugate or eflornithine-aspirin salt or a composition thereof into a subject.

The most common type of lung cancer is non-small cell lung cancer (NSCLC), which accounts for approximately 80-85% of lung cancers and is divided into squamous cell carcinomas, adenocarcinomas, and large cell undifferentiated carcinomas. Small cell lung cancer accounts for 15-20% of lung cancers.

Treatment options for lung cancer include surgery, immunotherapy, radiation therapy, chemotherapy, photodynamic therapy, or a combination thereof. Some possible surgical options for treatment of lung cancer are a segmental or wedge resection, a lobectomy, or a pneumonectomy. Radiation therapy can be external beam radiation therapy or brachytherapy.

Some anti-cancer drugs that can be used in chemotherapy to treat lung cancer include cisplatin, carboplatin, paclitaxel, docetaxel, gemcitabine, vinorelbine, irinotecan, etoposde, vinblastine, gefitinib, ifosfamide, methotrexate, or a combination thereof. Photodynamic therapy (PDT) can be used to treat lung cancer patients.

The methods provided by the invention can provide a beneficial effect for lung cancer patients, by administration of an eflornithine-NSAID conjugate or eflornithine-aspirin salt or eflornithine ester-aspirin salt or a composition thereof, and surgery, radiation therapy, chemotherapy, photodynamic therapy, or a combination thereof.

### Skin Cancer

In some embodiments, the invention provides methods to treat skin cancer. In some embodiments, the method comprises administering an eflornithine-NSAID conjugate or eflornithine-aspirin salt or eflornithine ester-aspirin salt or a composition thereof a subject.

There are several types of cancer that start in the skin. The most common types are basal cell carcinoma and squamous cell carcinoma, which are non-melanoma skin cancers. Actinic keratosis is a skin condition that sometimes develops into squamous cell carcinoma. Non-melanoma skin cancers rarely spread to other parts of the body. Melanoma, the rarest form of skin cancer, is more likely to invade nearby tissues and spread to other parts of the body. Different types of treatment are available for patients with non-melanoma and melanoma skin cancer and actinic keratosis including surgery, radiation therapy, chemotherapy and photodynamic therapy. Some possible surgical options for treatment of skin cancer are mohs micrographic surgery, simple excision, electrodesiccation and curettage, cryosurgery, laser surgery. Radiation therapy can be external beam radiation therapy or brachytherapy. Other types of treatments that are being tested in clinical trials are biologic therapy or immunotherapy, chemoimmunotherapy, topical chemotherapy with fluorouracil and photodynamic therapy.

The methods provided by the invention can provide a beneficial effect for skin cancer patients, by administration of an eflornithine-NSAID conjugate or eflornithine-aspirin salt or eflornithine ester-aspirin salt or a composition thereof, and surgery, radiation therapy, chemotherapy, photodynamic therapy, or a combination thereof.

### Eye Cancer, Retinoblastoma

In some embodiments, the invention provides methods to treat eye retinoblastoma. In some embodiments, the method comprises administering an eflornithine-NSAID conjugate or eflornithine-aspirin salt or a composition thereof into a subject.

Retinoblastoma is a malignant tumor of the retina. Although retinoblastoma can occur at any age, it most often occurs in younger children, usually before the age of 5 years. The tumor can be in one eye only or in both eyes. Retinoblastoma is usually confined to the eye and does not spread to nearby tissue or other parts of the body. Treatment options that attempt to cure the patient and preserve vision include enucleation (surgery to remove the eye), radiation therapy, cryotherapy, photocoagulation, immunotherapy, thermotherapy and chemotherapy. Radiation therapy can be external beam radiation therapy or brachytherapy.

The methods provided by the invention can provide a beneficial effect for eye retinoblastoma patients, by administration of an eflornithine-NSAID conjugate or eflornithine-aspirin salt or eflornithine ester-aspirin salt or a composition thereof, and surgery, radiation therapy, cryotherapy, photocoagulation, thermotherapy and chemotherapy, or a combination thereof.

### Eye Cancer, Intraocular Melanoma

In some embodiments, the invention provides methods to treat intraocular (eye) melanoma. In some embodiments, the method comprises administering an eflornithine-NSAID conjugate or eflornithine-aspirin salt or eflornithine ester-aspirin salt or a composition thereof, into a subject.

Intraocular melanoma, a rare cancer, is a disease in which cancer cells are found in the part of the eye called the uvea. The uvea includes the iris, the ciliary body, and the choroid. Intraocular melanoma occurs most often in people who are middle aged. Treatments for intraocular melanoma include surgery, immunotherapy, radiation therapy and laser therapy. Surgery is the most common treatment of intraocular melanoma. Some possible surgical options are iridectomy, iridotrabeculectomy, iridocyclectomy, choroidectomy, enucleation and orbital exenteration. Radiation therapy can be external beam radiation therapy or brachytherapy. Laser therapy can be an intensely powerful beam of light to destroy the tumor, thermotherapy or photocoagulation.

The methods provided by the invention can provide a beneficial effect for intraocular melanoma patients, by administration of an eflornithine-NSAID conjugate or eflornithine-aspirin salt or eflornithine ester-aspirin salt or a composition thereof, and surgery, radiation therapy and laser therapy, or a combination thereof.

### Endometrium Cancer

In some embodiments, the invention provides methods to treat endometrium cancer. In some embodiments, the method comprises administering an eflornithine-NSAID conjugate or eflornithine-aspirin salt or a composition thereof into a subject.

Endometrial cancer is a cancer that starts in the endometrium, the inner lining of the uterus. Some of the examples of the cancer of uterus and endometrium include, but are not limited to, adenocarcinomas, adenoacanthomas, adenosquamous carcinomas, papillary serous adenocarcinomas, clear cell adenocarcinomas, uterine sarcomas, stromal sarcomas, malignant mixed mesodermal tumors, and leiomyosarcomas.

The methods provided by the invention can provide a beneficial effect for endometrium cancer patients, by administration of an eflornithine-NSAID conjugate or eflornithine-aspirin salt or eflornithine ester-aspirin salt or a composition thereof, and surgery, radiation therapy, chemotherapy, gene therapy, RNA therapy, adjuvant therapy, photodynamic therapy, antiangiogenesis therapy, and immunotherapy, or a combination thereof.

### Liver Cancer

In some embodiments, the invention provides methods to treat primary liver cancer (cancer that begins in the liver). In some embodiments, the method comprises administering an eflornithine-NSAID conjugate or eflornithine-aspirin salt or a composition thereof into a subject.

Primary liver cancer can occur in both adults and children. Different types of treatments are available for patients with primary liver cancer. These include surgery, immunotherapy, radiation therapy, chemotherapy and percutaneous ethanol injection. The types of surgery that can be used are cryosurgery, partial hepatectomy, total hepatectomy and radiofrequency ablation. Radiation therapy can be external beam radiation therapy, brachytherapy, radiosensitizers or radiolabel antibodies. Other types of treatment include hyperthermia therapy and immunotherapy.

The methods provided by the invention can provide a beneficial effect for liver cancer patients, by administration of an eflornithine-NSAID conjugate or eflornithine-aspirin salt or eflornithine ester-aspirin salt or a composition thereof, and surgery, radiation therapy, chemotherapy, percutaneous ethanol injection, hyperthemia therapy and immunotherapy, or a combination thereof.

### Kidney Cancer

In some embodiments, the invention provides methods to treat kidney cancer. In some embodiments, the method comprises administering an eflornithine-NSAID conjugate or eflornithine-aspirin salt or a composition thereof into a subject.

Kidney cancer (also called renal cell cancer or renal adenocarcinoma) is a disease in which malignant cells are found in the lining of tubules in the kidney. Kidney cancer can be treated by surgery, radiation therapy, chemotherapy and immunotherapy. Some possible surgical options to treat kidney cancer are partial nephrectomy, simple nephrectomy and radical nephrectomy. Radiation therapy can be external beam radiation therapy or brachytherapy. Stem cell transplant can be used to treat kidney cancer.

The methods provided by the invention can provide a beneficial effect for kidney cancer patients, by administration of an eflornithine-NSAID conjugate or eflornithine-aspirin salt or eflornithine ester-aspirin salt or a composition thereof, and surgery, radiation therapy, chemotherapy, immunotherapy and stem cell transplant, or a combination thereof.

### Thyroid Cancer

In some embodiments, the invention provides methods to treat thyroid cancer. In some embodiments, the method comprises administering an eflornithine-NSAID conjugate or eflornithine-aspirin salt or eflornithine ester-aspirin salt or a composition thereof into a subject.

Thyroid cancer is a disease in which cancer (malignant) cells are found in the tissues of the thyroid gland. The four main types of thyroid cancer are papillary, follicular, medullary and anaplastic. Thyroid cancer can be treated by surgery, immunotherapy, radiation therapy, hormone therapy and chemotherapy. Surgery is the most common treatment of thyroid cancer. Some possible surgical options for treatment of thyroid cancer are lobectomy, near-total thyroidectomy, total thyroidectomy and lymph node dissection. Radiation therapy can be external radiation therapy or can required intake of a liquid that contains radioactive iodine. Hormone therapy uses hormones to stop cancer cells from growing. In treating thyroid cancer, hormones can be used to stop the body from making other hormones that might make cancer cells grow.

The methods provided by the invention can provide a beneficial effect for thyroid cancer patients, by administration of an eflornithine-NSAID conjugate or eflornithine-aspirin salt or eflornithine ester-aspirin salt or a composition thereof, and surgery, surgery, radiation therapy, hormone therapy and chemotherapy, or a combination thereof.

### AIDS-Related Cancers

### AIDS-Related Lymphoma

In some embodiments, the invention provides methods to treat AIDS-related lymphomas. The method comprises administering an eflornithine-NSAID conjugate or eflornithine-aspirin salt or a composition thereof into a subject.

AIDS-related lymphoma is a disease in which malignant cells form in the lymph system of patients who have acquired immunodeficiency syndrome (AIDS). AIDS is caused by the human immunodeficiency virus (HIV), which attacks and weakens the body's immune system. The immune system is then unable to fight infection and diseases that invade the body. People with HIV disease have an increased risk of developing infections, lymphoma, and other types of cancer. Lymphomas are cancers that affect the white blood cells of the lymph system. Lymphomas are divided into two general types: Hodgkin's lymphoma and non-Hodgkin's lymphoma. Both Hodgkin's lymphoma and non-Hodgkin's lymphoma can occur in AIDS patients, but non-Hodgkin's lymphoma is more common When a person with AIDS has non-Hodgkin's lymphoma, it is called an AIDS-related lymphoma. Non-Hodgkin's lymphomas can be indolent (slow-growing) or aggressive (fast-growing). AIDS-related lymphoma is usually aggressive. The three main types of AIDS-related lymphoma are diffuse large B-cell lymphoma, B-cell immunoblastic lymphoma and small non-cleaved cell lymphoma.

Treatment of AIDS-related lymphoma combines treatment of the lymphoma with treatment for AIDS. Patients with AIDS have weakened immune systems and treatment can cause further damage. For this reason, patients who have AIDS-related lymphoma are usually treated with lower doses of drugs than lymphoma patients who do not have AIDS. Highly-active antiretroviral therapy (HAART) is used to slow progression of HIV. Medicine to prevent and treat infections, which can be serious, is also used. AIDS-related lymphomas can be treated by chemotherapy, immunotherapy, radiation therapy and high-dose chemotherapy with stem cell transplant. Radiation therapy can be external beam radiation therapy or brachytherapy. AIDS-related lymphomas can be treated by monoclonal antibody therapy.

The methods provided by the invention can provide a beneficial effect for AIDS-related lymphoma patients, by administration of an eflornithine-NSAID conjugate or eflornithine-aspirin salt or eflornithine ester-aspirin salt or a composition thereof, and radiation therapy, or a combination thereof.

### Kaposi's Sarcoma

In some embodiments, the invention provides methods to treat Kaposi's sarcoma. The method comprises administering an eflornithine-NSAID conjugate or eflornithine-aspirin salt or eflornithine ester-aspirin salt or a composition thereof into a subject.

Kaposi's sarcoma is a disease in which cancer cells are found in the tissues under the skin or mucous membranes that line the mouth, nose, and anus. Classic Kaposi's sarcoma usually occurs in older men of Jewish, Italian, or Mediterranean heritage. This type of Kaposi's sarcoma progresses slowly, sometimes over 10 to 15 years. Kaposi's sarcoma can occur in people who are taking immunosuppressants. Kaposi's sarcoma in patients who have Acquired Immunodeficiency Syndrome (AIDS) is called epidemic Kaposi's sarcoma. Kaposi's sarcoma in people with AIDS usually spreads more quickly than other kinds of Kaposi's sarcoma and often is found in many parts of the body. Kaposi's sarcoma can be treated with surgery, chemotherapy, radiation therapy and immunotherapy. External radiation therapy is a common treatment of Kaposi's sarcoma. Some possible surgical options to treat Kaposi's Sarcoma are local excision, electrodeiccation and curettage, and cryotherapy.

The methods provided by the invention can provide a beneficial effect for Kaposi's sarcoma, by administration of an eflornithine-NSAID conjugate or eflornithine-aspirin salt or a composition thereof, and surgery, chemotherapy, radiation therapy and immunotherapy, or a combination thereof.

### Viral-Induced Cancers

In some embodiments, the invention provides methods to treat viral-induced cancers. Several common viruses are clearly or probable causal factors in the etiology of specific malignancies. These viruses either normally establish latency or few can become persistent infections. Oncogenesis is probably linked to an enhanced level of viral activation in the infected host, reflecting heavy viral dose or compromised immune control. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer. In general, these malignancies occur relatively early in life, typically peaking in middle-age or earlier.

### Virus-Induced Hepatocellular Carcinoma

The causal relationship between both HBV and HCV and hepatocellular carcinoma or liver cancer is established through substantial epidemiologic evidence. Both appear to act via chronic replication in the liver by causing cell death and subsequent regeneration. Different types of treatments are available for patients with liver cancer. These include surgery, immunotherapy, radiation therapy, chemotherapy and percutaneous ethanol injection. The types of surgery that can be used are cryosurgery, partial hepatectomy, total hepatectomy and radiofrequency ablation. Radiation therapy can be external beam radiation therapy, brachytherapy, radiosensitizers or radiolabel antibodies. Other types of treatment include hyperthermia therapy and immunotherapy.

The methods provided by the invention can provide a beneficial effect for virus induce hepatocellular carcinoma patients, by administration of an eflornithine-NSAID conjugate or eflornithine-aspirin salt or eflornithine ester-aspirin salt or a composition thereof, and radiation therapy, or a combination thereof.

### Viral-Induced Adult T Cell Leukemia/Lymphoma

The association between HTLV-1 and Adult T cell leukemia (ATL) is firmly established. Unlike the other oncogenic viruses found throughout the world, HTLV-1 is highly geographically restricted, being found primarily in southern Japan, the Caribbean, west and central Africa, and the South Pacific islands. Evidence for causality includes the monoclonal integration of viral genome in almost all cases of ATL in carriers. The risk factors for HTLV-1-associated malignancy appear to be perinatal infection, high viral load, and being male sex.

Adult T cell leukemia is a cancer of the blood and bone marrow. The standard treatments for adult T cell leukemia/lymphoma are radiation therapy, immunotherapy, and chemotherapy. Radiation therapy can be external beam radiation therapy or brachytherapy. Other methods of treating adult T cell leukemia/lymphoma include immunotherapy and high-dose chemotherapy with stem cell transplantation.

The methods provided by the invention can provide a beneficial effect for Adult T cell leukemia patients, by administration of an eflornithine-NSAID conjugate or eflornithine-aspirin salt or eflornithine ester-aspirin salt or a composition thereof, and radiation therapy, chemotherapy, immunotherapy and high-dose chemotherapy with stem cell transplantation, or a combination thereof.

### Viral-Induced Cervical Cancer

Infection of the cervix with human papillomavirus (HPV) is the most common cause of cervical cancer. Not all women with HPV infection, however, will develop cervical cancer. Cervical cancer usually develops slowly over time. Before cancer appears in the cervix, the cells of the cervix go through changes known as dysplasia, in which cells that are not normal begin to appear in the cervical tissue. Later, cancer cells start to grow and spread more deeply into the cervix and to surrounding areas. The standard treatments for cervical cancers are surgery, immunotherapy, radiation therapy and chemotherapy. The types of surgery that can be used are conization, total hysterectomy, bilateral salpingo-oophorectomy, radical hysterectomy, pelvic exenteration, cryosurgery, laser surgery and loop electrosurgical excision procedure. Radiation therapy can be external beam radiation therapy or brachytherapy.

The methods provided by the invention can provide a beneficial effect for adult cervical cancer, by administration of an eflornithine-NSAID conjugate or eflornithine-aspirin salt or eflornithine ester-aspirin salt or a composition thereof, and radiation therapy, chemotherapy, or a combination thereof.

### CNS Cancers

In some embodiments, the invention provides methods to treat central nervous system cancers. In some embodiments, the method comprises administering an eflornithine-NSAID conjugate or eflornithine-aspirin salt or eflornithine ester-aspirin salt or a composition thereof into a subject.

Brain and spinal cord tumors are abnormal growths of tissue found inside the skull or the bony spinal column, which are the primary components of the central nervous system (CNS). Benign tumors are noncancerous, and malignant tumors are cancerous. The CNS is housed within rigid, bony quarters (i.e., the skull and spinal column), so any abnormal growth, whether benign or malignant, can place pressure on sensitive tissues and impair function. Tumors that originate in the brain or spinal cord are called primary tumors. Most primary tumors are caused by out-of-control growth among cells that surround and support neurons. In a small number of individuals, primary tumors can result from specific genetic disease (e.g., neurofibromatosis, tuberous sclerosis) or from exposure to radiation or cancer-causing chemicals. The cause of most primary tumors remains a mystery.

The first test to diagnose brain and spinal column tumors is a neurological examination. Special imaging techniques (computed tomography, and magnetic resonance imaging, positron emission tomography) are also employed. Laboratory tests include the EEG and the spinal tap. A biopsy, a surgical procedure in which a sample of tissue is taken from a suspected tumor, helps doctors diagnose the type of tumor.

Tumors are classified according to the kind of cell from which the tumor seems to originate. The most common primary brain tumor in adults comes from cells in the brain called astrocytes that make up the blood-brain barrier and contribute to the nutrition of the central nervous system. These tumors are called gliomas (astrocytoma, anaplastic astrocytoma, or glioblastoma multiforme) and account for 65% of all primary central nervous system tumors. Some of the tumors are, but not limited to, Oligodendroglioma, Ependymoma, Meningioma, Lymphoma, Schwannoma, and Medulloblastoma.

### Neuroepithelial Tumors of the CNS

Astrocytic tumors, such as astrocytoma,; anaplastic (malignant) astrocytoma, such as hemispheric, diencephalic, optic, brain stem, cerebellar; glioblastoma multiforme; pilocytic astrocytoma, such as hemispheric, diencephalic, optic, brain stem, cerebellar; subependymal giant cell astrocytoma; and pleomorphic xanthoastrocytoma. Oligodendroglial tumors, such as oligodendroglioma; and anaplastic (malignant) oligodendroglioma. Ependymal cell tumors, such as ependymoma; anaplastic ependymoma; myxopapillary ependymoma; and subependymoma. Mixed gliomas, such as mixed oligoastrocytoma; anaplastic (malignant) oligoastrocytoma; and others (e.g. ependymo-astrocytomas). Neuroepithelial tumors of uncertain origin, such as polar spongioblastoma; astroblastoma; and gliomatosis cerebri. Tumors of the choroid plexus, such as choroid plexus papilloma; and choroid plexus carcinoma (anaplastic choroid plexus papilloma). Neuronal and mixed neuronal-glial tumors, such as gangliocytoma; dysplastic gangliocytoma of cerebellum (Lhermitte-Duclos); ganglioglioma; anaplastic (malignant) ganglioglioma; desmoplastic infantile ganglioglioma, such as desmoplastic infantile astrocytoma; central neurocytoma; dysembryoplastic neuroepithelial tumor; olfactory neuroblastoma (esthesioneuroblastoma. Pineal Parenchyma Tumors, such as pineocytoma; pineoblastoma; and mixed pineocytoma/pineoblastoma. Tumors with neuroblastic or glioblastic elements (embryonal tumors), such as medulloepithelioma; primitive neuroectodermal tumors with multipotent differentiation, such as medulloblastoma; cerebral primitive neuroectodermal tumor; neuroblastoma; retinoblastoma; and ependymoblastoma.

### Other CNS Neoplasms

Tumors of the Sellar Region, such as pituitary adenoma; pituitary carcinoma; and craniopharyngioma. Hematopoietic tumors, such as primary malignant lymphomas; plasmacytoma; and granulocytic sarcoma. Germ Cell Tumors, such as germinoma; embryonal carcinoma; yolk sac tumor (endodermal sinus tumor); choriocarcinoma; teratoma; and mixed germ cell tumors. Tumors of the Meninges, such as meningioma; atypical meningioma; and anaplastic (malignant) meningioma. Non-menigothelial tumors of the meninges, such as Benign Mesenchymal; Malignant Mesenchymal; Primary Melanocytic Lesions; Hemopoietic Neoplasms; and Tumors of Uncertain Histogenesis, such as hemangioblastoma (capillary hemangioblastoma). Tumors of Cranial and Spinal Nerves, such as schwannoma (neurinoma, neurilemoma); neurofibroma; malignant peripheral nerve sheath tumor (malignant schwannoma), such as epithelioid, divergent mesenchymal or epithelial differentiation, and melanotic. Local Extensions from Regional Tumors; such as paraganglioma (chemodectoma); chordoma; chodroma; chondrosarcoma; and carcinoma. Metastatic tumours, Unclassified Tumors and Cysts and Tumor-like Lesions, such as Rathke cleft cyst; Epidermoid; dermoid; colloid cyst of the third ventricle; enterogenous cyst; neuroglial cyst; granular cell tumor (choristoma, pituicytoma); hypothalamic neuronal hamartoma; nasal glial herterotopia; and plasma cell granuloma.

Chemotherapeutics available are, but not limited to, alkylating agents such as, Cyclophosphamide, Ifosphamide, Melphalan, Chlorambucil, BCNU, CCNU, Decarbazine, Procarbazine, Busulfan, and Thiotepa; antimetabolites such as, Methotraxate, 5-Fluorouracil, Cytarabine, Gemcitabine (Gemzar®), 6-mercaptopurine, 6-thioguanine, Fludarabine, and Cladribine; anthracyclins such as, daunorubicin. Doxorubicin, Idarubicin, Epirubicin and Mitoxantrone; antibiotics such as, Bleomycin; eflornithines such as, irinotecan and topotecan; taxanes such as, paclitaxel and docetaxel; and platinums such as, Cisplatin, carboplatin, and Oxaliplatin.

The treatments are surgery, radiation therapy, immunotherapy, hyperthermia, gene therapy, RNA therapy, adjuvant therapy, chemotherapy, and combination of radiation and chemotherapy. Doctors also can prescribe steroids to reduce the swelling inside the CNS.

The methods provided by the invention can provide a beneficial effect for CNS cancer, by administration of an eflornithine-NSAID conjugate or eflornithine-aspirin salt or eflornithine ester-aspirin salt or a composition thereof, and radiation therapy, chemotherapy, or a combination thereof.

### Colon Cancer and Rectal Cancer

In some embodiments, the invention provides methods to treat colorectal cancers. In some embodiments, the method comprises administering an eflornithine-NSAID conjugate or eflornithine-aspirin salt or eflornithine ester-aspirin salt or a composition thereof into a subject.

Colorectal cancer includes cancerous growths in the colon, rectum and appendix. Many colorectal cancers are thought to arise from adenomatous polyps in the colon. Colorectal cancer originates from the epithelial cells lining the gastrointestinal tract. Hereditary or somatic mutations in specific DNA sequences, among which are included DNA replication or DNA repair genes, and also the APC, K-Ras, NOD2 and p53 genes, lead to unrestricted cell division. Therapy is usually through surgery, which in many cases is followed by chemotherapy. Bacillus Calmette-Guérin (BCG) is being investigated as an adjuvant mixed with autologous tumor cells in immunotherapy for colorectal cancer.

Over 20% of patients present with metastatic (stage IV) colorectal cancer at the time of diagnosis, and up to 25% of this group have isolated liver metastasis that is potentially resectable. Patients with colon cancer and metastatic disease to the liver can be treated in either a single surgery or in staged surgeries depending upon the fitness of the patient for prolonged surgery, the difficulty expected with the procedure with either the colon or liver resection, and the comfort of the surgery performing potentially complex hepatic surgery.

The methods provided by the invention can provide a beneficial effect for colorectal cancer patients, by administration of an eflornithine-NSAID conjugate or eflornithine-aspirin salt or eflornithine ester-aspirin salt or a composition thereof, and radiation therapy, immunotherapy, or a combination thereof.

### Familial Adenomatous Polyposis Syndrome

Familial Adenomatous Polyposis (FAP), an inherited polyposis syndrome, is the result of germ-line mutation of the adenomatous polyposis coli (APC) tumor suppressor gene (Su et al., 1992). This autosomal-dominant condition with variable expression is associated with the development of hundreds of colonic adenomas, which uniformly progress to adenocarcinoma by forty years of age, two decades earlier than the mean age diagnosis for sporadic colon cancer (Bussey, 1990). In prior studies of pre-symptomatic individuals with FAP, increased levels of the polyamines spermidine and spermine, and their diamine precursor putrescine, have been detected in normal-appearing colorectal biopsies when compared to normal family member controls (Giardiello et al., 1997). The activity of ornithine decarboxylase (ODC), the first and rate-limiting enzyme in mammalian polyamine synthesis, also is elevated in apparently normal colonic mucosal biopsies from FAP patients (Giardiello et al., 1997; Luk and Baylin, 1984). These findings are of interest as the polyamines are necessary for optimal cell proliferation (Pegg, 1986). Further, suppression of ODC activity, using the enzyme-activated irreversible inhibitor DFMO, inhibits colon carcinogenesis in carcinogen-treated rodents (Kingsnorth et al., 1983; Tempero et al., 1989).

The Min (multiple intestinal neoplasia) mouse, which shares a mutated APC/apc genotype with FAP, serves as a useful experimental animal model for human FAP patients (Lipkin, 1997). The Min mouse can develop greater than 100 gastrointestinal adenomas/adenocarcinomas throughout the gastrointestinal tract by 120 days of life leading to GI bleeding, obstruction and death.

### Stomach Cancer

In some embodiments, the invention provides methods to treat stomach cancers. In some embodiments, the method comprises administering an eflornithine-NSAID conjugate or eflornithine-aspirin salt or eflornithine ester-aspirin salt or a composition thereof into a subject.

Stomach or gastric cancer can develop in any part of the stomach and can spread throughout the stomach and to other organs; particularly the esophagus and the small intestine. There are three main types of stomach cancers: lymphomas, gastric stromal tumors, and carcinoid tumors. Lymphomas are cancers of the immune system tissue that are sometimes found in the wall of the stomach. Gastric stromal tumors develop from the tissue of the stomach wall. Carcinoid tumors are tumors of hormone-producing cells of the stomach. Infection with the bacterium H. pylori is the main risk factor in about 80% or more of gastric cancers. It is more common in men. The causes of stomach cancer continue to be debated. A combination of heredity and environment (diet, smoking, etc) are all thought to play a part.

Common approaches to the treatment include surgery, immunotherapy, chemotherapy, radiation therapy, combination of chemotherapy and radiation therapy or biological therapy. Stomach cancer is difficult to cure unless it is found in an early stage (before it has begun to spread). New treatment approaches such as biological therapy and improved ways of using current methods are being studied in clinical trials.

The methods provided by the invention can provide a beneficial effect for stomach cancer patients, by administration of an eflornithine-NSAID conjugate or eflornithine-aspirin salt or eflornithine ester-aspirin salt or a composition thereof, and radiation therapy, immunotherapy, or a combination thereof.

### Gallbladder Cancer

In some embodiments, the invention provides methods to treat gallbladder cancers. In some embodiments, the method comprises administering an eflornithine-NSAID conjugate or eflornithine-aspirin salt or eflornithine ester-aspirin salt or a composition thereof into a subject.

Gallbladder cancer is a rare cancer in which malignant cells are found in the tissues of the gallbladder. The gallbladder stores bile, a fluid made by the liver to digest fat. The wall of the gallbladder has 3 main layers of tissue: mucosal (innermost) layer, muscularis (middle, muscle) layer, and serosal (outer) layer. Between these layers is supporting connective tissue. Primary gallbladder cancer starts in the innermost layer and spreads through the outer layers as it grows. Gallbladder cancer can be cured only if it is found before it has spread, when it can be removed by surgery. If the cancer has spread, palliative treatment can improve the patient's quality of life by controlling the symptoms and complications of this disease.

The methods provided by the invention can provide a beneficial effect for gallbladder cancer patients, by administration of an eflornithine-NSAID conjugate or eflornithine-aspirin salt or eflornithine ester-aspirin salt or a composition thereof, and radiation therapy, immunotherapy, or a combination thereof.

### Esophageal Cancer

In other embodiments, the invention provides methods to treat esophageal cancers. In some embodiments, the method comprises administering an eflornithine-NSAID conjugate or eflornithine-aspirin salt or eflornithine ester-aspirin salt or a composition thereof into a subject.

Esophageal cancer is malignancy of the esophagus. There are various subtypes. Most tumors of the esophagus are malignant. A very small proportion (under 10%) is leiomyoma (smooth muscle tumor) or gastrointestinal stromal tumor (GIST). Malignant tumors are generally adenocarcinomas, squamous cell carcinomas, and occasionally small-cell carcinomas. The latter share many properties with small-cell lung cancer, and are relatively sensitive to chemotherapy compared to the other types.

Small and localized tumors are treated surgically with curative intent. Larger tumors tend not to be operable and hence cannot be cured; their growth can still be delayed with chemotherapy, radiotherapy or a combination of the two. In some cases chemo- and radiotherapy can render these larger tumors operable.

The methods provided by the invention can provide a beneficial effect for esophageal cancer patients, by administration of an eflornithine-NSAID conjugate or eflornithine-aspirin salt or a composition thereof, and radiation therapy, immunotherapy, or a combination thereof.

### PNS Cancers

In some embodiments, the invention provides methods to treat peripheral nervous system (PNS) cancers. In some embodiments, the method comprises administering an eflornithine-NSAID conjugate or eflornithine-aspirin salt or eflornithine ester-aspirin salt or a composition thereof into a subject.

The peripheral nervous system consists of the nerves that branch out from the brain and spinal cord. These nerves form the communication network between the CNS and the body parts. The peripheral nervous system is further subdivided into the somatic nervous system and the autonomic nervous system. The somatic nervous system consists of nerves that go to the skin and muscles and is involved in conscious activities. The autonomic nervous system consists of nerves that connect the CNS to the visceral organs such as the heart, stomach, and intestines. It mediates unconscious activities.

Acoustic neuromas are benign fibrous growths that arise from the balance nerve, also called the eighth cranial nerve or vestibulocochlear nerve. These tumors are non-malignant, meaning that they do not spread or metastasize to other parts of the body. The location of these tumors is deep inside the skull, adjacent to vital brain centers in the brain stem. As the tumors enlarge, they involve surrounding structures which have to do with vital functions. In the majority of cases, these tumors grow slowly over a period of years.

The malignant peripheral nerve sheath tumor (MPNST) is the malignant counterpart to benign soft tissue tumors such as neurofibromas and schwannomas. It is most common in the deep soft tissue, usually in close proximity of a nerve trunk. The most common sites include the sciatic nerve, brachial plexus, and sarcal plexus. The most common symptom is pain which usually prompts a biopsy. It is a rare, aggressive, and lethal orbital neoplasm that usually arises from sensory branches of the trigeminal nerve in adults. Malignant PNS tumor spreads along nerves to involve the brain, and most patients die within 5 years of clinical diagnosis. The MPNST can be classified into three major categories with epithelioid, mesenchymal or glandular characteristics. Some of the MPNST include but not limited to, Subcutaneous malignant epithelioid schwannoma with cartilaginous differentiation, Glandular malignant schwannoma, Malignant peripheral nerve sheath tumor with perineurial differentiation, Cutaneous epithelioid malignant nerve sheath tumor with rhabdoid features, Superficial epithelioid MPNST, Triton Tumor (MPNST with rhabdomyoblastic differentiation), Schwannoma with rhabdomyoblastic differentiation. Rare MPNST cases contain multiple sarcomatous tissue types, especially osteosarcoma, chondrosarcoma and angiosarcoma. These have sometimes been indistinguishable from the malignant mesenchymoma of soft tissue.

Other types of PNS cancers include but not limited to, malignant fibrous cytoma, malignant fibrous histiocytoma, malignant meningioma, malignant mesothelioma, and malignant mixed Müllerian tumor.

The treatments are surgery, radiation therapy, immunotherapy, chemotherapy, and combination of radiation and chemotherapy.

The methods provided by the invention can provide a beneficial effect for PNS cancer patients, by administration of an eflornithine-NSAID conjugate or eflornithine-aspirin salt or eflornithine ester-aspirin salt or a composition thereof, and radiation therapy, immunotherapy, or a combination thereof.

### Head and Neck, Oral Cavity and Oropharyngeal Cancer

In other embodiments, the invention provides methods to treat head and neck cancers including cancers of the lip, oral cavity, nasal cavity, paranasal sinuses, pharynx, and larynx. In some embodiments, the method comprises administering an eflornithine-NSAID conjugate or eflornithine-aspirin salt or eflornithine ester-aspirin salt or a composition thereof into a subject.

Cancers such as, hypopharyngeal cancer, laryngeal cancer, nasopharyngeal cancer, oropharyngeal cancer, and the like, have been treated with surgery, immunotherapy, chemotherapy, combination of chemotherapy and radiation therapy. Etoposide and actinomycin D, two commonly used oncology agents that inhibit topoisomerase II, fail to cross the blood-brain barrier in useful amounts.

The methods provided by the invention can provide a beneficial effect for oral cavity and oropharyngeal cancer, by administration of an eflornithine-NSAID conjugate or eflornithine-aspirin salt or eflornithine ester-aspirin salt or a composition thereof, and radiation therapy, immunotherapy, or a combination thereof.

### Testicular Cancer

In another aspect, the invention provides methods to treat testicular cancer. In some embodiments, the method comprises administering an eflornithine-NSAID conjugate or eflornithine-aspirin salt or eflornithine ester-aspirin salt or a composition thereof into a subject.

Testicular cancer is cancer that typically develops in one or both testicles in young men. Cancers of the testicle develop in certain cells known as germ cells. The 2 main types of germ cell tumors (GCTs) that occur in men are seminomas (60%) and nonseminomas (40%). Tumors can also arise in the supportive and hormone-producing tissues, or stroma, of the testicles. Such tumors are known as gonadal stromal tumors. The 2 main types are Leydig cell tumors and Sertoli cell tumors. Secondary testicular tumors are those that start in another organ and then spread to the testicle. Lymphoma is the most common secondary testicular cancer.

Common approaches to the treatment include surgery, immunotherapy, chemotherapy, radiation therapy, combination of chemotherapy and radiation therapy or biological therapy. Several drugs are typically used to treat testicular cancer: Platinol (cisplatin), Vepesid or VP-16 (etoposide) and Blenoxane (bleomycin sulfate). Additionally, Ifex (ifosamide), Velban (vinblastine sulfate) and others can be used.

The methods provided by the invention can provide a beneficial effect for stomach cancer, by administration of an eflornithine-NSAID conjugate or eflornithine-aspirin salt or eflornithine ester-aspirin salt or a composition thereof, and radiation therapy, chemotherapy, or a combination thereof.

### Thymus Cancer

In other embodiments, the invention provides methods to treat thymus cancer. In some embodiments, the method comprises administering an eflornithine-NSAID conjugate or eflornithine-aspirin salt or eflornithine ester-aspirin salt or a composition thereof into a subject.

The thymus is a small organ located in the upper/front portion of your chest, extending from the base of the throat to the front of the heart. The thymus contains 2 main types of cells, thymic epithelial cells and lymphocytes. Thymic epithelial cells can give origin to thymomas and thymic carcinomas. Thymomas are epithelial tumors of the thymus, which may or may not be extensively infiltrated by nonneoplastic lymphocytes. The term thymoma is customarily used to describe neoplasms that show no overt atypia of the epithelial component. A thymic epithelial tumor that exhibits clear-cut cytologic atypia and histologic features no longer specific to the thymus is known as a thymic carcinoma (also known as type C thymoma). Lymphocytes, whether in the thymus or in the lymph nodes, can become malignant and develop into cancers called Hodgkin disease and non-Hodgkin lymphomas. The thymus also contains another much less common type of cells called Kulchitsky cells, or neuroendocrine cells, which normally release certain hormones. These cells can give rise to cancers, called carcinoids or carcinoid tumors that often release the same type of hormones, and are similar to other tumors arising from neuroendocrine cells elsewhere in the body.

Common approaches to the treatment include surgery, immunotherapy, chemotherapy, radiation therapy, combination of chemotherapy and radiation therapy or biological therapy. Anticancer drugs that have been used in the treatment of thymomas and thymic carcinomas are doxorubicin (Adriamycin), cisplatin, ifosfamide, and corticosteroids (prednisone). Often, these drugs are given in combination to increase their effectiveness. Combinations used to treat thymic cancer include cisplatin, doxorubicin, etoposide and cyclophosphamide, and the combination of cisplatin, doxorubicin, cyclophosphamide, and vincristine.

The methods provided by the invention can provide a beneficial effect for stomach cancer, by administration of an eflornithine-NSAID conjugate or eflornithine-aspirin salt or a composition thereof, and radiation therapy, chemotherapy, or a combination thereof.

### Urethral Cancer

In some embodiments, the invention provides methods to treat urethral cancer. In some embodiments, the method comprises administering an eflornithine-NSAID conjugate or eflornithine-aspirin salt or eflornithine ester-aspirin salt or a composition thereof into a subject.

Urethral cancer is a rare cancer that occurs more often in women than in men. There are different types of urethral cancer that begin in cells that line the urethra. These cancers are named for the types of cells that become malignant: Squamous cell carcinoma is the most common type of urethral cancer. It forms in cells in the part of the urethra near the bladder in women, and in the lining of the urethra in the penis in men. Transitional cell carcinoma forms in the area near the urethral opening in women, and in the part of the urethra that goes through the prostate gland in men. Adenocarcinoma forms in glands near the urethra in both men and women.

Treatment of urethral cancer depends on the stage of the cancer and where it is in the urethra; the patient's sex and general health; and whether the cancer has just been diagnosed or has recurred.

The methods provided by the invention can provide a beneficial effect for urethral cancer patients, by administration of an eflornithine-NSAID conjugate or eflornithine-aspirin salt or a composition thereof, and radiation therapy, immunotherapy, or a combination thereof.

Sarcomas Other than Kaposi's Sarcoma

In some embodiments, the invention provides methods to treat sarcomas other than Kaposi's sarcoma. In some embodiments, the method comprises administering an eflornithine-NSAID conjugate or eflornithine-aspirin salt or a composition thereof into a subject.

There are several subtypes of sarcomas, based on the type of tissue from which they arise. For example, osteosarcoma arises from bone, chondrosarcoma arises from cartilage, and leiomyosarcoma arises from smooth muscle. Soft tissue sarcomas, such as leiomyosarcoma, chondrosarcoma, and gastrointestinal stromal tumor (GIST), are more common in adults than in children. Bone sarcomas, such as osteosarcoma and Ewing's sarcoma, are more common in children than in adults. These tumors most commonly strike adolescents and young adults between the ages of 12 and 25. In addition to being named based on the tissue of origin, sarcomas are also assigned a grade, such as low grade or high grade. Low grade sarcomas are usually treated surgically, although sometimes radiation therapy or chemotherapy is used. High grade sarcomas are more frequently treated with chemotherapy. Since these tumors are more likely to undergo metastasis, these tumors are treated more aggressively. Childhood sarcomas are almost always treated with a combination of surgery and chemotherapy, and radiation is frequently used as well. The recognition that childhood sarcomas are sensitive to chemotherapy has dramatically improved the survival of patients.

### Vaginal Cancer

Vaginal cancer is a disease in which malignant cells form in the vagina. Carcinomas of the vagina include squamous cell carcinoma, adenocarcinoma, melanoma and sarcoma. Squamous cell vaginal carcinoma spreads slowly and usually stays near the vagina, but may spread to the lungs and liver. Adenocarcinoma begins in glandular (secretory) cells. Adenocarcinoma is more likely than squamous cell cancer to spread to the lungs and lymph nodes.

### Cancer Stem Cells

In some embodiments, methods and compositions of the present invention are used to treat cancers derived from cancer stem cells. Cancer stem cells (CSCs) are a sub-population of cancer cells found within tumors or hematological cancers that possess characteristics normally associated with stem cells. CSCs are believed to be tumorigenic, in contrast to the bulk of cancer cells, which are thought to be non-tumorigenic. CSCs have stem cell properties such as self-renewal and the ability to differentiate into multiple cell types. CSCs are also capable of forming heterogeneous tumors in immunodeficient mice at high frequency. It has been suggested that CSCs persist in tumors as a distinct population and cause relapse and metastasis by giving rise to new tumors. Most human tumors have now been shown to contain a sub-population of cells that display cancer stem cell characteristics. The types of cancer include but are not limited to leukemia, breast cancer, melanoma, lung cancer, brain cancers, colon cancers, pancreatic cancer, and ovarian cancer.

The existence of cancer stem cells has several implications in terms of cancer treatment and therapies. Normal stem cells are naturally resistant to chemotherapeutic agents because they have various pumps (such as MDR) that pump out drugs. Stem cells also have DNA repair proteins and a slow rate of cell turnover. Cancer stem cells, being the mutated counterparts of normal stem cells, can also have similar functions which allow them to survive various therapies. By selectively targeting cancer stem cells, it would be possible to treat patients with aggressive tumors, as well as preventing the tumor from metastasizing. References on cancer stem cells and cancer stem cell targeted agents include Trumpp A, Wiestler O D. Mechanisms of Disease: cancer stem cells—targeting the evil twin. Nat Clin Pract Oncol. 2008 June; 5(6):337-47. Epub 2008 April 22. Chumsri S, Burger A M. Cancer stem cell targeted agents: therapeutic approaches and consequences. Curr Opin Mol Ther. 2008 August; 10(4):323-33, both of which are herein incorporated by reference in their entireties.

The methods provided by the invention can provide a beneficial effect for cancer patients, by administration of an eflornithine-NSAID conjugate or eflornithine-aspirin salt or eflornithine ester-aspirin salt or a composition thereof, and radiation therapy, RNA therapy, adjuvant therapy, nanotherapy, gene therapy, immunotherapy, or a combination thereof.

The present invention of eflornithine-NSAID conjugates or eflornithine-aspirin salts provides pharmaceutical advantages of particular use in medicine. First, these eflornithine-NSAID conjugates are typically labile in vivo, cleaved by either enzymatic or chemical pathway, to generate substantial quantities of eflornithine analogs and the NSAIDs upon reaching the systemic circulation and tumor cells. Second, the eflornithine analogs, and the other NSAIDs, upon in vivo release, typically target two or more biological targets that are relevant for cancer treatment. The linkers released from the conjugates are typically non-toxic when administered to a mammal with dosing regimens typically comparable to the co-administration of eflornithine analogs and the NSAIDs. Third, some of the NSAIDs selected are hydrophobic. As a result, the final eflornithine-NSAID conjugates are more hydrophilic than the parent NSAID analogs. Therefore, the final drug conjugates have better water solubility than the parent NSAID derivatives.

### Administration

In some embodiments, the eflornithine-NSAID conjugates or eflornithine-aspirin salts or eflornithine ester-aspirin salt and/or compositions of the present invention are administered or applied singly, in combination with one or more pharmaceutically active agents, including but not limited to other compounds of the invention.

The present eflornithine-NSAID conjugates or eflornithine-aspirin salts or eflornithine ester-aspirin salt and/or compositions thereof are preferably administered via oral administration. The eflornithine-NSAID conjugates or eflornithine-aspirin salts or eflornithine ester-aspirin salt and/or compositions thereof can also be administered via any parenteral route, for example, by infusion or bolus injection, by absorption through epithelial or mucocutaneous linings (e.g. oral mucosa, rectal and intestinal mucosa, etc.). Administration can be systemic or local. Various delivery systems are known for use in administering an eflornithine-NSAID conjugate or eflornithine-aspirin salt or eflornithine ester-aspirin salt and/or composition thereof, for example, encapsulation in liposomes, microparticles, microcapsules, capsules, etc. Methods of administration include, but are not limited to, intradermal, intramuscular, intraperitoneal, intravenous, subcutaneous, intranasal, epidural, oral, sublingual, intracerebral, intravaginal, transdermal, rectally, inhalation, topically, particularly to the ears, nose, eyes, or skin, as described in Section IV hereinabove.

In some preferred embodiments, a sustained release formulation is utilized as described in Section IV. The system restrains the eflornithine-NSAID conjugate or eflornithine-aspirin salt or eflornithine ester-aspirin salt by incorporating, encapsulating, surrounding, or entrapping the compound in, on or by lipid vesicles or liposomes, or by micelles. The liposome bound prodrug can preferably intercalate between the acyl chains of the lipid.

The eflornithine-NSAID conjugates of the invention can be cleaved either chemically and/or enzymatically. One or more enzymes present in the stomach, intestinal lumen, intestinal tissue, blood, liver, brain or any other suitable tissue of a mammal can enzymatically cleave the linkers of the eflornithine-NSAID conjugates. The mechanism of cleavage of eflornithine-NSAID conjugate can be one that is known in the art or one that is unknown or novel to the relevant field. The linkers of the eflornithine-NSAID conjugates can be cleaved prior to absorption by the gastrointestinal tract or after absorption by the gastrointestinal tract (e.g. in intestinal tissue, blood, liver or other suitable tissue of a mammal). If the linkers of the eflornithine-NSAID conjugates are cleaved prior to absorption by the gastrointestinal tract, the drugs and the eflornithine analogs can be absorbed into the systemic circulation conventionally by active transport and/or passive diffusion.

In some embodiments, a patient is treated by direct injection of a tumor or its vasculature with the therapeutic compounds. Alternatively, the tumor can be infused or perfused with the therapeutic compounds using any suitable delivery vehicle. Local or regional administration, with respect to the tumor, also is contemplated. Finally, systemic administration can be performed. Continuous administration also can be applied where appropriate, for example, where a tumor is excised and the tumor bed is treated to eliminate residual, microscopic disease. Delivery via syringe or catheterization is preferred. Such continuous perfusion can take place for a period from about 1-2 hours, to about 2-6 hours, to about 6-12 hours, to about 12-24 hours, to about 1-2 days, to about 1-2 wk or longer following the initiation of treatment. Generally, the dose of the therapeutic composition via continuous perfusion will be equivalent to that given by a single or multiple injections, adjusted over a period of time during which the perfusion occurs.

### Dosage

Pharmaceutical compositions suitable for use in the present invention include compositions wherein the active ingredients are present in an effective amount, i.e., in an amount effective to achieve therapeutic and/or prophylactic benefit in a host with a cancer or at least one symptom of a cancer. The actual amount effective for a particular application will depend on the condition or conditions being treated, the condition of the subject, the severity of the affliction, the formulation, and the route of administration, as well as other factors known to those of skill in the art. In vitro or in vivo assays can optionally be employed to help identify the optimal dosage ranges. Determination of an effective amount of an eflornithine-NSAID conjugate or eflornithine-aspirin salt or eflornithine ester-aspirin salt is well within the capabilities of those skilled in the art, in light of the disclosure herein, and will be determined using routine optimization techniques.

The effective amount for use in humans can be determined from animal models. For example, a dose for humans can be formulated to achieve circulating, liver, topical and/or gastrointestinal concentrations that have been found to be effective in animals. The eflornithine-NSAID conjugates or eflornithine-aspirin salts or eflornithine ester-aspirin salt are preferably tested in at least one animal model to demonstrate safety and efficacy. In some embodiments, a therapeutically effective dose of an eflornithine-NSAID conjugate or eflornithine-aspirin salt or eflornithine ester-aspirin salt described herein provides therapeutic benefit without causing substantial toxicity while providing synergistic effect as compared to each individual compound dosed or two parent compounds co-formulated. Toxicity of eflornithine-NSAID conjugates or eflornithine-aspirin salts or eflornithine ester-aspirin salt can be determined using standard pharmaceutical procedures and can be readily ascertained by the skilled artisan. The dose ratio between toxic and therapeutic effect is the therapeutic index. In some embodiments, an eflornithine-NSAID conjugate or eflornithine-aspirin salt or eflornithine ester-aspirin salt exhibits higher therapeutic indices in treating a type of cancer mentioned herein as compared to their parent compounds. The dosage of an eflornithine-NSAID conjugate or eflornithine-aspirin salt or eflornithine ester-aspirin salt is within a range of circulating concentrations that result in little or no toxicity. One skilled in the art can determine the effective amount for human use, especially in light of the animal model experiments described herein. Based on animal data, and other types of similar data, those skilled in the art can determine the effective amounts of compositions of the present invention appropriate for humans.

The effective amount when referring to an eflornithine-NSAID conjugate or eflornithine-aspirin salt or a combination of the eflornithine-NSAID conjugates or eflornithine-aspirin salts or eflornithine ester-aspirin salt with other anti-tumor drugs or therapies will generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

In some embodiments, the therapeutically effective amount of eflornithine-NSAID conjugate or eflornithine-aspirin salt or eflornithine ester-aspirin salt of the invention is the amount of the compound which inhibits or retards the growth of a cancer cell, kills a cancer cell, induces apoptosis or cell cycle arrest of a cancer cell, causes regression and palliation of a malignant tumor, i.e., reduces the volume or size of such tumor or eliminate the tumor entirely, delays progression of cancer, prevents or delays metastasis, decreases symptoms resulting from cancer, increases the quality of life of those suffering from cancer, decreases the dose of other medications or biologically active agents required to treat cancer, enhances the effect of another biologically active agent or therapy for treating cancer, and/or prolongs survival of the subject with cancer.

In a mammal such as a human, the effective amount can be determined on the basis of body surface area. The inter-relationship of dosages varies among animals of various sizes and species. For humans, the dosage is based on mg/m² of body surface (E. J. Freireich, et al., Cancer Chemother. Rep. 1966, 50(4), 219). Body surface area can be approximately determined from the height and weight of an individual (Scientific Table, Geigy Pharmaceuticals, Ardsley, N.Y. pp. 537-538, 1970). In some embodiments, a suitable dose range of an eflornithine-NSAID conjugate or eflornithine-aspirin salt or eflornithine ester-aspirin salt is from about 1 to about 1000 mg per m² of body surface area. In further embodiments, the dose range is from 10 to 500 mg/m² for a human being. It should be noted that the suitable dosage range of the eflornithine-NSAID conjugate or eflornithine-aspirin salt or eflornithine ester-aspirin salt is also dependent on the potency of the parent eflornithine analog and the NSAID.

In some embodiments, administration of eflornithine-NSAID conjugates or eflornithine-aspirin salts is intermittent, for example administration once every two days, every three days, every five days, once a week, once or twice a month, and the like. In some embodiments, the amount, forms, and/or amounts of the different forms vary at different times of administration to achieve the optimal clinical results. For instance, when intra-lipid 20 is used as the formulation, the actual dosage of the eflornithine-NSAID conjugate or eflornithine-aspirin salt or eflornithine ester-aspirin salt that reaches the patient will be less due to loss of certain amount of the drug conjugate or eflornithine-aspirin salt or eflornithine ester-aspirin salt on the walls of the syringe or preparation vessel. In other embodiments, if cotton seed oil is used, the loss of the compound is not so prevalent. The dosage of a pharmaceutical composition of the present invention can be delivered by a single administration, multiple applications or a controlled release. Dosing can be repeated intermittently, provided alone or in combination with other drugs. The schedule can continue for as long as an effective treatment of cancer demands.

In one embodiment, the active eflornithine analog upon release from the conjugate or eflornithine-aspirin salt or eflornithine ester-aspirin salt is present in an amount sufficient to exert an anti-tumor activity, preferably less than the amount normally used when administered alone, and the active NSAID, upon release from the conjugate, is present in an amount, that results in less or reduced side effects such as gastrointestinal damage otherwise caused by the NSAID when administered alone. Yet both the eflornithine analog and the NSAID, in their active forms upon in vivo release from the conjugate, are present at a therapeutically effective dose that results in a superior therapeutic efficacy against cancer cells as compared to when the eflornithine analog and the NSAID are administered alone or co-formulated.

A person skilled in the art would be able to monitor in a patient the effect of administration of an eflornithine-NSAID conjugate or eflornithine-aspirin salt or eflornithine ester-aspirin salt. For example, tumor response can be determined by techniques standard in the art.

### VI. Methods of Use

### A. Combination Therapy

In certain embodiments of the present invention, the eflornithine-NSAID conjugates or eflornithine-aspirin salts or eflornithine ester-aspirin salt of the invention can be used in a combination therapy with at least one other therapeutic agent. The eflornithine-NSAID conjugates or eflornithine-aspirin salts or eflornithine ester-aspirin salt of the invention and the therapeutic agent can act additively or, more preferably, synergistically.

Combination therapy includes the administration of a conjugate or salt of the invention and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually minutes, hours, days or weeks depending upon the combination selected). Combination therapy can be carried out either sequentially or substantially simultaneously. In the case of sequential administration of more than one therapeutic agent, each therapeutic agent is administered at a different time. In the case of simultaneous administration, at least two of the therapeutic agents are administered in a substantially simultaneous manner, either in the same pharmaceutical composition or in different pharmaceutical compositions. Substantially simultaneous administration can be accomplished, for example, by administering to the subject a single capsule having a fixed ratio of each therapeutic agent or in multiple, single capsules for each of the therapeutic agents. In a preferred embodiment, a composition comprising an eflornithine-NSAID conjugate or eflornithine-aspirin salt or eflornithine ester-aspirin salt of the invention is administered concurrently with the administration of another therapeutic agent, which can be part of the same composition as the eflornithine-NSAID conjugate or eflornithine-aspirin salt or eflornithine ester-aspirin salt of the invention or a different composition. In another embodiment, a composition comprising an eflornithine-NSAID conjugate or eflornithine-aspirin salt or eflornithine ester-aspirin salt of the invention is administered prior to, or subsequent to, administration of another therapeutic agent.

Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination selected can be administered by intravenous injection while the other therapeutic agents of the combination can be administered orally. Alternatively, for example, all therapeutic agents can be administered orally or all therapeutic agents can be administered by intravenous injection. The sequence in which the therapeutic agents are administered is not narrowly critical.

Combination therapy also encompasses the administration of the eflornithine-NSAID conjugate or eflornithine-aspirin salt or eflornithine ester-aspirin salt as described above in further combination with other therapies including but not limited to surgery, radiation therapy, gene therapy, immunotherapy, RNA therapy, adjuvant therapy, nanotherapy or a combination thereof. Where the combination therapy further comprises a non-drug treatment, the non-drug treatment can be conducted at any suitable time so long as a beneficial effect from the co-action of the combination of the therapeutic agents and non-drug treatment is achieved. For example, in appropriate cases, the beneficial effect is still achieved when the non-drug treatment is temporally removed from the administration of the therapeutic agents, by a significant period of time. The conjugate or eflornithine-aspirin salt or eflornithine ester-aspirin salt and the other pharmacologically active agent can be administered to a patient simultaneously, sequentially or in combination. It will be appreciated that when using a combination of the invention, the compound of the invention and the other pharmacologically active agent can be in the same pharmaceutically acceptable carrier and therefore administered simultaneously. They can be in separate pharmaceutical carriers such as conventional oral dosage forms which are taken simultaneously. The term “combination” further refers to the case where the compounds are provided in separate dosage forms and are administered sequentially.

### Gene Therapy Agents

Gene therapy agents insert copies of genes into a specific set of a patient's cells, and can target both cancer and non-cancer cells. The goal of gene therapy can be to replace altered genes with functional genes, to stimulate a patient's immune response to cancer, to make cancer cells more sensitive to chemotherapy, to place “suicide” genes into cancer cells, or to inhibit angiogenesis. Genes can be delivered to target cells using viruses, liposomes, or other carriers or vectors. This can be done by injecting the gene-carrier composition into the patient directly, or ex vivo, with infected cells being introduced back into a patient. Such compositions are suitable for use in the present invention.

### Nanotherapy

Nanometer-sized particles have novel optical, electronic, and structural properties that are not available from either individual molecules or bulk solids. When linked with tumor-targeting moieties, such as tumor-specific ligands or monoclonal antibodies, these nanoparticles can be used to target cancer-specific receptors, tumor antigens (biomarkers), and tumor vasculatures with high affinity and precision. The formulation and manufacturing process for cancer nanotherapy is disclosed in U.S. Pat. No. 7,179,484, and article M. N. Khalid, P. Simard, D. Hoarau, A. Dragomir, J. Leroux, Long Circulating Poly(Ethylene Glycol)Decorated Lipid Nanocapsules Deliver Docetaxel to Solid Tumors, Pharmaceutical Research, 23(4), 2006, all of which are herein incorporated by reference in their entireties.

### RNA Therapy

RNA including but not limited to siRNA, shRNA, microRNA can be used to modulate gene expression and treat cancers. Double stranded oligonucleotides are formed by the assembly of two distinct oligonucleotide sequences where the oligonucleotide sequence of one strand is complementary to the oligonucleotide sequence of the second strand; such double stranded oligonucleotides are generally assembled from two separate oligonucleotides (e.g., siRNA), or from a single molecule that folds on itself to form a double stranded structure (e.g., shRNA or short hairpin RNA). These double stranded oligonucleotides known in the art all have a common feature in that each strand of the duplex has a distinct nucleotide sequence, wherein only one nucleotide sequence region (guide sequence or the antisense sequence) has complementarity to a target nucleic acid sequence and the other strand (sense sequence) comprises nucleotide sequence that is homologous to the target nucleic acid sequence.

MicroRNAs (miRNA) are single-stranded RNA molecules of about 21-23 nucleotides in length, which regulate gene expression. miRNAs are encoded by genes that are transcribed from DNA but not translated into protein (non-coding RNA); instead they are processed from primary transcripts known as pri-miRNA to short stem-loop structures called pre-miRNA and finally to functional miRNA. Mature miRNA molecules are partially complementary to one or more messenger RNA (mRNA) molecules, and their main function is to downregulate gene expression.

Certain RNA inhibiting agents can be utilized to inhibit the expression or translation of messenger RNA (“mRNA”) that is associated with a cancer phenotype. Examples of such agents suitable for use herein include, but are not limited to, short interfering RNA (“siRNA”), ribozymes, and antisense oligonucleotides. Specific examples of RNA inhibiting agents suitable for use herein include, but are not limited to, Cand5, Sirna-027, fomivirsen, and angiozyme.

### Adjuvant Therapy

Adjuvant therapy is a treatment given after the primary treatment to increase the chances of a cure. In some embodiments, adjuvant therapy includes but is not limited to chemotherapy, radiation therapy, hormone therapy, or biological therapy.

Because the principal purpose of adjuvant therapy is to kill any cancer cells that may have spread, treatment is usually systemic (uses substances that travel through the bloodstream, reaching and affecting cancer cells all over the body). For example, adjuvant therapy for breast cancer involves chemotherapy or hormone therapy, either alone or in combination.

Adjuvant chemotherapy is the use of drugs to kill cancer cells. For example, research has shown that using chemotherapy as adjuvant therapy for early stage breast cancer helps to prevent the original cancer from returning. Adjuvant chemotherapy is usually a combination of anticancer drugs, which has been shown to be more effective than a single anticancer drug.

### Small Molecule Enzymatic Inhibitors

Certain small molecule therapeutic agents are able to target the tyrosine kinase enzymatic activity or downstream signal transduction signals of certain cell receptors such as epidermal growth factor receptor (“EGFR”) or vascular endothelial growth factor receptor (“VEGFR”). Such targeting by small molecule therapeutics can result in anti-cancer effects. Examples of such agents suitable for use herein include, but are not limited to, imatinib, gefitinib, erlotinib, lapatinib, canertinib, ZD6474, sorafenib (BAY 43-9006), ERB-569, and their analogues and derivatives.

### Anti-Metastatic Agents

The process whereby cancer cells spread from the site of the original tumor to other locations around the body is termed cancer metastasis. Certain agents have anti-metastatic properties, designed to inhibit the spread of cancer cells. Examples of such agents suitable for use herein include, but are not limited to, marimastat, bevacizumab, trastuzumab, rituximab, erlotinib, MMI-166, GRN163L, hunter-killer peptides, tissue inhibitors of metalloproteinases (TIMPs), their analogues, derivatives and variants.

### Chemopreventative Agents

Certain pharmaceutical agents can be used to prevent initial occurrences of cancer, or to prevent recurrence or metastasis. Administration with such chemopreventative agents in combination with eflornithine-NSAID conjugates or eflornithine-aspirin salts or eflornithine ester-aspirin salt of the invention can act to both treat and prevent the recurrence of cancer. Examples of chemopreventative agents suitable for use herein include, but are not limited to, tamoxifen, raloxifene, tibolone, bisphosphonate, ibandronate, estrogen receptor modulators, aromatase inhibitors (letrozole, anastrozole), luteinizing hormone-releasing hormone agonists, goserelin, vitamin A, retinal, retinoic acid, fenretinide, 9-cis-retinoid acid, 13-cis-retinoid acid, all-trans-retinoic acid, isotretinoin, tretinoid, vitamin B6, vitamin B12, vitamin C, vitamin D, vitamin E, cyclooxygenase inhibitors, non-steroidal anti-inflammatory drugs (NSAIDs), aspirin, ibuprofen, celecoxib, polyphenols, polyphenol E, green tea extract, folic acid, glucaric acid, interferon-alpha, anethole dithiolethione, zinc, pyridoxine, finasteride, doxazosin, selenium, indole-3-carbinal, alpha-difluoromethylornithine, carotenoids, beta-carotene, lycopene, antioxidants, coenzyme Q10, flavonoids, quercetin, curcumin, catechins, epigallocatechin gallate, N-acetylcysteine, indole-3-carbinol, inositol hexaphosphate, isoflavones, glucanic acid, rosemary, soy, saw palmetto, and calcium. An additional example of chemopreventative agents suitable for use in the present invention is cancer vaccines. These can be created through immunizing a patient with all or part of a cancer cell type that is targeted by the vaccination process.

### B. Reduction of Adverse Effects and Enhancement of Therapeutic Efficacy

In another aspect, the present invention also provides a method for reducing an adverse effect and/or increasing therapeutic efficacy associated with a treatment of cancer by administering to a subject in need a therapeutically effective amount of an eflornithine-NSAID conjugate or eflornithine-aspirin salt or eflornithine ester-aspirin salt or a pharmaceutical composition thereof.

Toxicity and therapeutic efficacy of the conjugates or salts described herein can be determined by standard pharmaceutical procedures in experimental animals, e.g., by determining the IC₅₀ and the LD₅₀ (lethal dose causing death in 50% of the tested animals) for a subject compound.

In some embodiments, the composition of the invention reduces the incidence of unwanted side effects caused by various cancer treatment regimens including but not limited to hearing loss, dizziness, vertigo, thrombocytopenia, diarrhea, nausea, vomiting, weakness, fatigue, low blood cell count, hair loss, poor appetite, fever, weight loss, loss of overall mobility, cystitis, constipation, shortness of breath, insomnia, cough, headache, dehydration, chills, skin rash, flatulence, flushing, mouth sores, heartburn, swelling and inflammation.

### Side-Effect Limiting Agents

In some embodiments, treatment of cancer with eflornithine-NSAID conjugates or eflornithine-aspirin salts or eflornithine ester-aspirin salt is accompanied by administration of pharmaceutical agents that can alleviate the side effects produced by the antineoplastic agents. Such agents suitable for use herein include, but are not limited to, anti-emetics, anti-mucositis agents, pain management agents, infection control agents, and anti-anemia/anti-thrombocytopenia agents. Examples of anti-emetics suitable for use herein include, but are not limited to, 5-hydroxytryptamine 3 receptor antagonists, metoclopramide, steroids, lorazepam, ondansetron, cannabinoids, their analogues and derivatives. Examples of anti-mucositis agents suitable for use herein include, but are not limited to, palifermin (keratinocyte growth factor), glucagon-like peptide-2, teduglutide, L-glutamine, amifostin, and fibroblast growth factor 20. Examples of pain management agents suitable for use herein include, but are not limited to, opioids, opiates, and non-steroidal anti-inflammatory compounds. Examples of agents used for control of infection suitable for use herein include, but are not limited to, antibacterials such as aminoglycosides, penicillins, cephalosporins, tetracyclines, clindamycin, lincomycin, macrolides, vancomycin, carbapenems, monobactams, fluoroquinolones, sulfonamides, nitrofurantoins, their analogues and derivatives. Examples of agents that can treat anemia or thrombocytopenia associated with chemotherapy suitable for use herein include, but are not limited to, erythropoietin, and thrombopoietin.

In some embodiments, the composition of the present invention achieves adequate anti-tumor efficacy at a lower dose than that required for each individual unconjugated drug. In other embodiments, the composition of the present invention has improved pharmacokinetic and physiological properties including but not limited to improved aqueous solubility of the eflornithine analogs and improved absorption of the eflornithine analogs, allowing these drugs to reach their full potential in treatment of cancers. In yet other embodiments, the use of a sustained release formulation for delivery of the composition of the present invention further reduces the GI side effects caused by NSAIDs, thereby achieving better tumor targeting and optimal cancer prevention using the eflornithine-NSAID conjugates.

It is to be understood that the following examples are intended to be illustrative and not restrictive. Many embodiments will be apparent to those of skill in the art upon reading the above description. The scope of the invention should, therefore, be determined not with reference to the above description or the following examples, but should instead be determined with reference to appended claims, along with the full scope of equivalents to which such claims are entitled.

### EXAMPLES

### Example 1

### Synthesis of Eflornithine-Aspirin Conjugate

The present invention provides a method for synthesizing an eflornithine-NSAID conjugate. The example shown below generally describes the synthesis of an eflornithine-analog-NSAID conjugate, and more specifically, an eflornithine-aspirin conjugate.

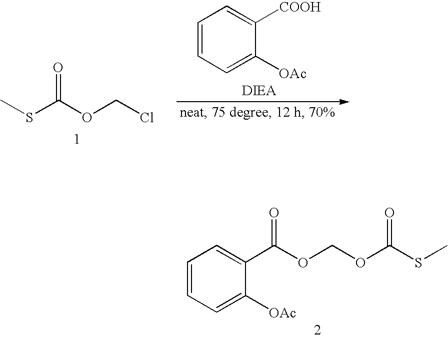

To the mixture of aspirin (0.72 g, 4 mmol) and diisopropylamine (0.22 g, 2 mmol) was added chloroalkylmethanethiocarbonates 1 (0.14 g, 1 mmol). The mixture was stirred at 75° C. for 12 hrs and cooled to room temperature. Then 10 mL methyl-tert-butyl ether (MTBE) and 10 mL water were added to the reaction mixture. The mixture was stirred and the organic phase was washed with saturated sodium carbonate solution (Na₂CO₃), 10% sodium hydroxide and brine and then dried over anhydrous sodium sulfate (Na₂SO₄). After the solvent was removed by rotary evaporation and the crude compound (2) was purified by silica gel column chromatography with 6:1 petrol ether (60-90° C.). The white solid product was obtained, mp: 86-88° C. ¹H NMR (600 MHz, CDCl₃) δ 8.08 (d, J=7.7 Hz, 1H), 7.62 (t, J=7.4 Hz, 1H), 7.34 (t, J=7.6 Hz, 1H), 7.14 (d, J=8.0 Hz, 1H), 6.01 (s, 2H), 2.39 (s, 3H), 2.37 (s, 3H).

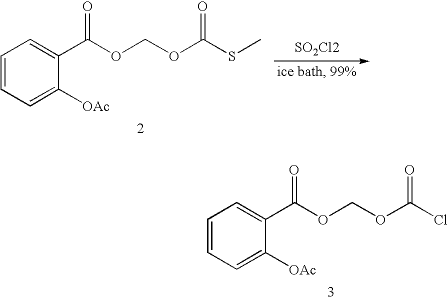

Redistilled SO₂Cl₂ (6.75 g, 50 mmol) was added to compound 2 (7.12 g, 25 mmol) at 0° C. The mixture was stirred for 2 h. Then 20 mL toluene was added and the solvent and SO₂Cl₂ were removed by rotary evaporation. Light yellow product 3 was obtained. ¹H NMR (400 MHz, CDCl₃) δ 8.07 (d, J=7.9 Hz, 1H), 7.63 (t, J=7.8 Hz, 1H), 7.35 (t, J=7.7 Hz, 1H), 7.14 (d, J=8.1 Hz, 1H), 6.00 (s, 2H), 2.36 (s, 3H).

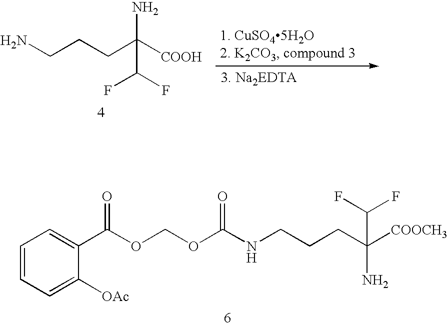

To a flask was added DL-eflornithine hydrochloride (437 mg, 2 mmol) and 1 M NaOH (2 mL, 2 mmol). To the result clear, colorless solution was added CuSO₄.5H₂O (500 mg, 1 mmol). After stirring for 2 h, K₂CO₃ (280 mg, 2 mmol) was added followed by compound 3 (544 mg, 2 mmol). After stirring over night at room temperature, the blue precipitate was collected and rinsed with Et₂O. The blue precipitate was added to a solution containing EDTA-Na₂.2H₂O (372 mg, 1 mmol) in 8 mL water. The resultant slurry was heated to 95° C. with vigorous stirring for 1 h then cooled to room temperature. After the solvent was removed by rotary evaporation, the crude compound was dissolved in EtOH and filtered. After the solvent was removed by rotary evaporation, compound 5 was obtained.

¹H NMR (600 MHz, D₂O) δ 8.06 (d, J=7.5 Hz, 1H), 7.75 (t, J=6.8 Hz, 1H), 7.48 (t, J=6.5 Hz, 1H), 7.26 (d, J=7.4 Hz, 1H), 6.30 (t, J=53.3 Hz, 1H), 5.92 (s, 2H), 3.23 (s, 2H), 2.40 (s, 3H), 2.05 (t, J=14.2 Hz, 1H), 1.86 (t, J=12.7 Hz, 1H), 1.68 (m, 1H), 1.51 (m, 1H). HRMS (ESI) found 441.1076 ([M+Na]⁺, calcd for C₁₇H₂₀F₂N₂O₈Na 441.1080).

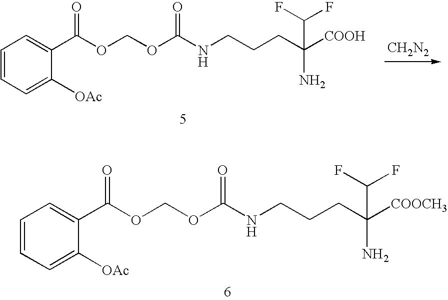

Compound 5 (627 mg, 1.5 mmol) dissolved in 10 mL MeOH was added to diazomethane (4 mmol) in 10 mL Et₂O at 0° C. The mixture was stirred for 4 h and the solvent was removed by rotary evaporation. The crude product was purified by silica gel column chromatography with 6:1 petrol ether (60-90° C.): ethyl acetate to obtain viscose liquid compound.

¹H NMR (600 MHz, CDCl₃) δ 8.09 (d, J=7.3 Hz, 1H), 7.60 (t, J=7.5 Hz, 1H), 7.33 (t, J=7.5 Hz, 1H), 7.12 (d, J=7.9 Hz, 1H), 5.92 (s, 2H), 5.86 (t, J=55.8 Hz, 1H), 5.23 (s, 1H), 3.78 (s, 3H), 3.30-3.14 (m, 2H), 2.37 (s, 3H), 1.86 (t, J=12.8 Hz, 1H), 1.77-1.62 (m, 3H), 1.58 (t, J=14.4 Hz, 1H), 1.44 (m, 1H). HRMS (ESI) found 455.1234 ([M+Na]⁺, calcd for C₁₈H₂₂F₂N₂O₈Na 455.1236).

### Example 2

### Synthesis of Eflornithine-Aspirin Salt

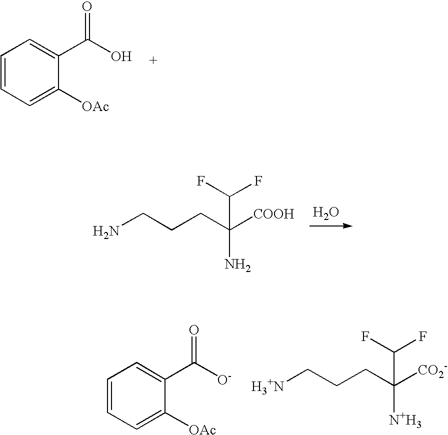

To eflornithine (0.9 g, 5 mmol) dissolved in 7 mL distilled water was added aspirin (0.9 g, 5 mmol). The mixture was stirred for 1 h at room temperature. The solution was filtered and the solvent was removed by rotary evaporation. The crude was rinsed with ethereal ether. White solid product was obtained, mp: 128-130° C. ¹H NMR (400 MHz, D₂O) δ 7.54 (d, J=7.6 Hz, 1H), 7.37 (t, J=7.7 Hz, 1H), 7.22 (t, J=7.5 Hz, 1H), 7.00 (d, J=8.1 Hz, 1H), 6.19 (t, J=53.5 Hz, 1H), 2.90 (t, J=7.5 Hz, 2H), 2.19 (s, 3H), 1.95 (td, J=13.7, 4.4 Hz, 1H), 1.73 (m, 2H), 1.59-1.45 (m, 1H).

### Example 3

### Synthesis of Eflornithine Prodrug

This example generally provides the synthesis of an eflornithine prodrug, and more specifically, a phosphoramidate prodrug.

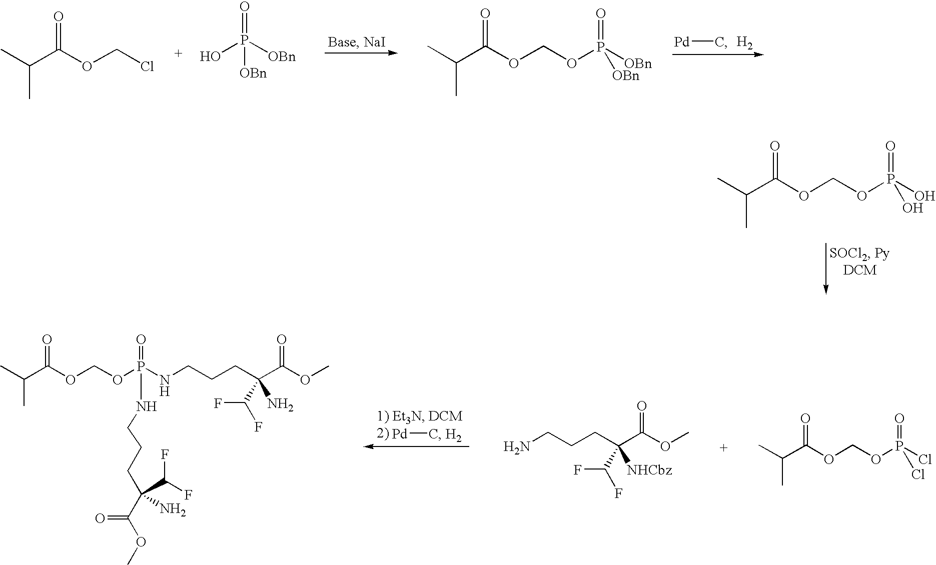

### Example 4

### In Vitro Determination of Caco-2 Cellular Permeability of Eflornithine Conjugates or Eflornithine Prodrug

The passive permeability of the eflornithine-NSAID conjugates or eflornithine prodrugs of the current invention is assessed in vitro using standard methods well known in the art (See, e.g., Stewart, et al., Pharm. Res., 1995, 12, 693). For example, passive permeability is evaluated by examining the flux of an eflornithine-NSAID conjugates or eflornithine prodrugs across a cultured polarized cell monolayer (e.g., Caco-2 cells). Caco-2 cells obtained from continuous culture (passage less than 28) are seeded at high density onto Transwell polycarbonate filters. Cells are maintained with DMEM/10% fetal calf serum +0.1 mM nonessential amino acids +2 mM L-Gln, 5% CO₂/95% O₂, 37° C. until the day of the experiment. Permeability studies are conducted at pH 6.5 apically (in 50 mM MES buffer containing 1 mM CaCl₂, 1 mM MgCl₂, 150 mM NaCl, 3 mM KCl, 1 mM NaH₂PO₄, 5 mM glucose) and pH 7.4 basolaterally (in Hanks' balanced salt solution containing 10 mM HEPES) in the presence of efflux pump inhibitors (250 μM MK-571, 250 uM Verapamil, 1 mM Ofloxacin). Inserts are placed in 12 or 24 well plates containing buffer and incubated for 30 min at 37C.°. Drug conjugate or prodrug (200 μM) is added to the apical or basolateral compartment (donor) and concentrations of drug conjugate or prodrug and/or released parent drug in the opposite compartment (receiver) are determined at intervals over 1 hour using LC/MS/MS. Values of apparent permeability (Pₐₚₚ) are calculated using the equation:

Pₐₚₚ=Vᵣ(dC/dt)/(ACₒ)

Here Vᵣ is the volume of the receiver compartment in mL; dC/dt is the total flux of eflornithine-NSAID conjugates or prodrugs and parent drugs (μM/s), determined from the slope of the plot of concentration in the receiver compartment versus time; Cₒ is the initial concentration of eflornithine-NSAID conjugates in μM; A is the surface area of the membrane in cm². Preferably, Eflornithine-NSAID conjugate with significant transcellular permeability demonstrate a value of Pₐₚₚ of ≧1×10⁻⁶ cm/s and more preferably, a value of Pₐₚₚ of ≧1×10⁻⁵ cm/s, and still more preferably a value of Pₐₚₚ of ≧5×10⁻⁵ cm/s.

### Example 5

### Chemical Stability

For the chemical stability studies, buffers are prepared at pH 2.0 (using 0.1M potassium phosphate and 0.5M NaCl), pH 7.4 and pH 8.0 (using 0.1M Tris-HCl and 0.5M NaCl). Compounds (5 μM) are incubated with buffers at 37° C. for 1 hour in a temperature controlled HPLC autosampler. Samples are injected at zero and 1 hour post-addition. Samples are analyzed by LC/MS/MS as described below.

### Example 6

### Metabolic Stability

Plasma Stability: Compounds (5 μM) are incubated with 90% rat or human plasma at 37° C. for 1 hour. Samples are obtained at zero and 1 hour post-addition and are immediately quench with methanol to prevent further conversion. Quenched samples are frozen and maintained at −80° C. prior analysis. Samples are analyzed by LC/MS/MS as described below.

Liver Homogenate: Compounds (5 μM) are incubated with rat or human liver S9 at 0.5 mg protein/mL in the presence of 1 mM NADPH at pH 7.4 and at 37° C. for 1 hour. Samples are obtained at zero and 1 hour post-addition and are immediately quench with methanol to prevent further conversion. Quenched samples are frozen and maintained at −80° C. prior analysis. Samples are analyzed by LC/MS/MS as described below.

Caco-2 Cell Homogenate: Caco-2 cells are grown in flasks over 21 days. Cells are then rinsed/scraped off into ice-cold 10 mM sodium phosphate/0.15 M potassium chloride, pH 7.4. Cells will be lysed by sonication at 4° C. using a probe sonicator and centrifuged at 9,000×g for 20 min at 4° C. and the resulting supernatant (Caco-2 cell homogenate S9 fraction) aliquots are transferred into 0.5 mL vials and stored at −80° C. prior to use. For stability studies, compounds (5 μM) are incubated with Caco-2 S9 (0.5 mg protein/mL) at pH 7.4 and 37° C. for 1 hr. Samples are obtained at zero and 1 hr post-addition and are immediately quenched with methanol to prevent further conversion. Quenched samples are frozen and maintained at −80° C. prior to analysis. Samples are analyzed by LC/MS/MS as described below.

Pancreatin: Compounds (5 μM) are incubated with porcine pancreatin (10 mg/mL in pH 7.5 buffer) at 37° C. for 1 hour. Samples are obtained at zero and 1 hour post-addition and are immediately quench with methanol to prevent further conversion. Quenched samples are frozen and maintained at −80° C. prior analysis. Samples are analyzed by LC/MS/MS as described below.

Drug Conjugates or Prodrugs Metabolism in Various Species: The drug conjugate (10 μM) is incubated with plasma, intestinal S9, lung S9, liver S9 and kidney S9 from rats, dogs, monkeys and humans at 37° C. for 1 hour. All preparations should contain 1 mg protein/mL. Samples are obtained at zero and intervals over 1 hour post addition and are immediately quenched with methanol to prevent further conversion. Quenched samples are then frozen and maintained at −80° C. prior to analysis. Samples are analyzed by LC/MS/MS as described below. The rate of conversion of drug conjugates to parent drugs in each matrix is calculated in pmol/min/mg protein.

Inhibition of Specific CYP450 Isoforms by Drug Conjugates: The lead candidate of Eflornithine-NSAID conjugate or eflornithine prodrug is tested for inhibition of CYP450 isoforms. The ability of drug conjugate to inhibit cytochrome P450-mediated metabolism is examined by standard methods using specific CYP450 isoforms expressed in bacculosomes (Supersomes™). The experimental conditions for each isoform are listed below. Standard substrates are employed that generate fluorescent metabolites. Experiments are conducted in a 96 well format. All incubations included an NADPH cofactor mix. The final concentration of CYP450 protein in each incubation should be between 2.5 to 5.0 μM. All compounds including positive control compounds are serially diluted in the solution of NADPH generation system to give final concentration of up to 400 μM. The resulting solutions are incubated with a specific CYP450 isoform and the related substrate at 37° C. for 15 to 45 minutes. A stop solution (80% acetonitrile/20% 0.5 M Tris base) is added to terminate the reaction. The samples are analyzed using a FlexStation fluorescence plate reader.

The percent inhibition of the formation of product is determined for each drug conjugate concentration and for control inhibitors. Blank values are subtracted from the sample wells to obtain the net fluorescence signal. The concentrations of drug conjugate that bracketed 50% inhibition (C_{High} and C_{Low}) are determined. The IC₅₀ values for inhibition of each specific isoform are then determined from the bracketing concentrations and corresponding percent inhibition values via linear interpolation as follows:

IC₅₀=(50%−% I_{Low})/(% I_{High}−% I_{Low})×(C_{High}−C_{Low})+C_{Low}

where C_{Low} and C_{High} are the concentrations bracketing 50% inhibition and % I_{High} and % I_{Low} are the corresponding percent inhibition values at the low and high concentrations, respectively. This is the calculation method recommended by the supplier of the Supersomes™.

CYP Isoforms (Standard Substrate): CYP3A4 (7-benzyloxytrifluoromethycoumarin); CYP1A2 (3-cyano-7-ethoxycoumarin); CYP2C9 (7-methoxytrifluoromethylcoumarin); CYP2C19 (3-cyano-7-ethoxycoumarin); CYP2D6 (3-[2-(N,N-diethyl-N-methylamino)ethyl]-7-methyoxy-4-methylcoumarin); CYP2E1 (7-methyoxy-4-trifluoromethylcoumarin).

### Example 7

### Uptake of Eflornithine Analogs and Conjugated NSAIDs Following Administration of Eflornithine, NSAIDs, Eflornithine-NSAIDs Conjugates and Eflornithine Prodrugs Intracolonically in Rats

Sustained release oral dosage forms, which release drug slowly over periods of 6-24 hours, generally release a significant proportion of the dose within the colon. Thus, drugs suitable for use in such dosage forms preferably exhibit good colonic absorption. This experiment is conducted to assess the suitability of eflornithine-NSAID conjugates for use in an oral sustained release formulation.

### Step A: Administration Protocol

Rats are obtained commercially and are pre-cannulated in the both the ascending colon and the jugular vein. Animals should be conscious at the time of the experiment. All animals are fasted overnight and until 4 hours post-dosing. The compounds of the interest are administered as a solution (in water or can be other solvent such as PEG 400) directly into the colon via the cannula at a dose of the desire. Blood samples (0.5 mL) are obtained from the jugular cannula at intervals over 8 hours and are quenched immediately by addition of acetonitrile/methanol to prevent further conversion of the eflornithine-NSAID conjugates or eflornithine prodrugs. Blood samples are analyzed as described below.

### Step B: Sample Preparation for Colonic Absorbed Drug

In blank 1.5 mL eppendorf tubes, 300 μL of 50/50 acetonitrile/methanol and 20 μL of p-chlorophenylalanine are added as an internal standard.
1. Rat blood is collected at different time points and immediately 100 μL of blood is added into the eppendorf tube and vortexed to mix.
2. 10 μL of an eflornithine or a NSAID standard solution (0.04, 0.2, 1, 5, 25, 100 μg/mL) is added to 90 μL of blank rat blood to make up a final calibration standard (0.004, 0.02, 0.1, 0.5, 2.5, 10 μg/mL). Then 300 μL of 50/50 acetonitrile/methanol is added into each tube followed by 20 μL of p-chlorophenylalanine.
3. Samples are vortexed and centrifuged at 14,000 rpm for 10 min.
4. Supernatant is taken for LC/MS/MS analysis.

### Step C: LC/MS/MS Analysis

A LC/MS/MS spectrometer equipped with 10ADVp binary pumps and a CTC HTS-PAL autosampler is used in the analysis. A column of the choice is heated to 45° C. during the analysis. The mobile phase can be different solvent mixtures, such as 0.1% formic acid (A) and acetonitrile with 0.1% formic acid (B). The gradient condition can be varied depend on the compound analyzed. A TurbolonSpray source can be used on the LC/MS/MS instrument such as API 2000. The analysis can be done in both positive or negative ion mode and an MRM transition can be selected based on the analysis of the compounds. 20 μL of the samples is injected. The peaks can be integrated using Analyst 1.1 quantitation software. Following colonic administration of each of these eflornithine-NSAID conjugates or prodrugs, the maximum plasma concentrations of eflornithine and NSAIDs (Cₘₐₓ), as well as the area under the eflornithine and NSAIDs plasma concentration vs. time curves (AUC) are compared to the parent drugs. A desired conjugates or prodrugs should provide both eflornithine and NSAIDs with higher Cₘₐₓ and greater AUC values than eflornithine and NSAID itself. This data demonstrates that compounds of the invention can be formulated as compositions suitable for enhanced absorption and/or effective sustained release of eflornithine and NSAIDs chosen to minimize dosing frequency due to rapid systemic clearance of eflornithine.

### Example 8

### Pharmacokinetics of Conjugated Eflornithine Analogs or Conjugated NSAIDs Following Intravenous Administration to Cynomolgus Monkeys

Eflornithine-NSAID conjugates are administered to four male cynomolgus monkeys as an aqueous solution by intravenous bolus injection into the saphenous vein at a desired dose. Blood samples are obtained from all animals at intervals over 24 hours post-dosing. Blood is processed immediately for plasma at 4° C. All plasma samples are subsequently analyzed for eflornithine or NSAIDs using the LC/MS/MS assay described above.

### Example 9

### Uptake of Eflornithine or Conjugated NSAIDs Following Administration of Eflornithine or Eflornithine-NSAID Conjugates Intracolonically in Cynomolgus Monkeys

Eflornithine, NSAIDs and eflornithine-NSAID conjugates are administered at a desired dose to groups of four male cynomolgus monkeys as either aqueous solutions or suspensions via bolus injection directly into the colon via an indwelling cannula. For colonic delivery, a flexible French catheter is inserted into the rectum of each monkey and extended to the proximal colon (approx. 16 inches) using fluoroscopy. Monkeys are lightly sedated by administration of Telazol/ketamine during dosing. A washout period of at least 5 to 7 days is allowed between treatments. Following dosing, blood samples are obtained at intervals over 24 hours and are immediately quenched and processed for plasma at 4° C. All plasma samples are subsequently analyzed for eflornithine, NSAIDs and intact eflornithine-NSAID conjugates using the LC/MS/MS assay described above. Following colonic administration of eflornithine-NSAIDs conjugates, the maximum plasma concentrations of eflornithine and NSAIDs (Cₘₐₓ), as well as the area under eflornithine and NSAIDs plasma concentration vs. time curves (AUC) are significantly greater than that produced from colonic administration of eflornithine itself. This data demonstrates that these eflornithine-NSAID conjugates can be formulated as compositions suitable for enhanced absorption and/or effective sustained release of eflornithine to minimize dosing frequency due to rapid systemic clearance of eflornithine.

### Example 10

### Uptake of Eflornithine and Conjugated NSAIDs Following Oral Administration of Eflornithine-NSAIDs Conjugates to Cynomolgus Monkeys

The eflornithine-NSAIDs conjugates are administered by oral gavage to groups of four male cynomolgus monkeys as either an aqueous solution or suspension respectively. Following dosing, blood samples are obtained at intervals over 24 hours and are immediately quenched and processed for plasma at 4° C. All plasma samples are subsequently analyzed for eflornithine, NSAIDs and intact eflornithine-NSAID conjugates using the LC/MS/MS assay described above.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound for treating or preventing cancer, the compound comprising a first moiety and a second moiety, the first moiety being covalently linked to the second moiety, wherein the first moiety is eflornithine or an analog or derivative of eflornithine, and the second moiety is a non-steroidal anti-inflammatory drug (NSAID).

2. The compound of claim 1, wherein the first moiety is eflornithine.

3. The compound of claim 1, wherein the second moiety is selected from the group consisting of aspirin, aceclofenac, acemethacin, alclofenac, amoxiprin, ampyrone, azapropazone, benorylate, bromfenac, choline and magnesium salicylates, choline salicylate, celecoxib, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, rofecoxib, salsalate, salicyl salicylate, salicylamide, sodium salicylate, sulindac, tiaprofenic acid, tolfenamic acid, tolmetin sodium, and valdecoxib.

4. The compound of claim 1, wherein the NSAID is sulindac.

5. The compound of claim 1, wherein the NSAID is aspirin.

6. The compound of claim 1, wherein the first and second moieties are linked via a covalent bond selected from the group consisting of an ester bond, an amide bond, an imine bond, a carbamate bond, a carbonate bond, a thioester bond, an acyloxycarbamate bond, an acyloxycarbonate bond, an acyloxythiocarbamate, a phosphate bond, a phosphoramidate and an acyloxyphosphate bond.

7. The compound of claim 1 further comprises a linker that covalently links the first moiety to the second moiety.

8. The compound of claim 7, wherein the linker is physiologically labile.

9. The compound of claim 1, wherein the cancer is adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarcinoma, head and neck cancers, teratocarcinoma, or Waldenstrom's macroglobulinemia.

10. The compound of claim 1, wherein the cancer is a Ki-ras-dependent cancer.

11. The compound of claim 1, wherein the compound can be used in combination with at least one other therapeutic agent.

12. The compound of claim 11, wherein the other therapeutic agent is an antitumor alkylating agent, antitumor antimetabolite, antitumor antibiotics, plant-derived antitumor agent, antitumor organoplatinum compound, antitumor campthotecin derivative, antitumor tyrosine kinase inhibitor, monoclonal antibody, interferon, biological response modifier, hormonal anti-tumor agent, angiogenesis inhibitor, differentiating agent, or a pharmaceutically acceptable salt thereof.

13. The compound of claim 1, wherein the compound can be used in combination with surgery, radiation therapy, chemotherapy, gene therapy, RNA therapy, adjuvant therapy, immunotherapy, nanotherapy or a combination thereof.

14. A pharmaceutical composition for treating or preventing cancer, comprising the compound of claim 1 and a pharmaceutically acceptable carrier.

15. A method of treating or preventing cancer, the method comprising administering to a subject in need thereof a therapeutically effective amount of a compound, the compound comprising a first moiety and a second moiety, the first moiety being covalently linked to the second moiety, wherein the first moiety is eflornithine or an analog or derivative of eflornithine, and the second moiety is a non-steroidal anti-inflammatory drug (NSAID).

16. The method of claim 15, wherein the first moiety is eflornithine.

17. The method of claim 15, wherein the second moiety is selected from the group consisting of aspirin, aceclofenac, acemethacin, alclofenac, amoxiprin, ampyrone, azapropazone, benorylate, bromfenac, choline and magnesium salicylates, choline salicylate, celecoxib, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, rofecoxib, salsalate, salicyl salicylate, salicylamide, sodium salicylate, sulindac, tiaprofenic acid, tolfenamic acid, tolmetin sodium, and valdecoxib.

18. The method of claim 15, wherein the NSAID is sulindac.

19. The method of claim 15, wherein the NSAID is aspirin.

20. The method of claim 15, wherein the first and second moieties are linked via a covalent bond selected from the group consisting of an ester bond, an amide bond, an imine bond, a carbamate bond, a carbonate bond, a thioester bond, an acyloxycarbamate bond, an acyloxycarbonate bond, a phosphate bond, a phosphoramidate bond and an acyloxyphosphate bond.

21. The method of claim 15, wherein the compound further comprises a linker that covalently links the first moiety to the second moiety.

22. The method of claim 21, wherein the linker is physiologically labile.

23. The method of claim 15, wherein the cancer is adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarcinoma, head and neck cancers, teratocarcinoma, or Waldenstrom's macroglobulinemia.

24. The method of claim 15, wherein the cancer is a Ki-ras-dependent cancer.

25. The method of claim 15 further comprising administering to the subject at least one other therapeutic agent.

26. The method of claim 25, wherein the other therapeutic agent is an antitumor alkylating agent, antitumor antimetabolite, antitumor antibiotics, plant-derived antitumor agent, antitumor organoplatinum compound, antitumor campthotecin derivative, antitumor tyrosine kinase inhibitor, monoclonal antibody, interferon, biological response modifier, hormonal anti-tumor agent, angiogenesis inhibitor, differentiating agent, or a pharmaceutically acceptable salt thereof.

27. The method of claim 25, wherein the other therapeutic agent is administered prior to, concomitant with or subsequent to administering the compound.

28. The method of claim 15, wherein the compound is administered in combination with surgery, radiation therapy, chemotherapy, gene therapy, RNA therapy, adjuvant therapy, immunotherapy, nanotherapy or a combination thereof.

29. The method of claim 15 further comprises administering to the subject a therapeutically effective amount of a pharmaceutical composition, the composition comprising the compound and a pharmaceutically acceptable carrier.

30. The method of claim 15, wherein the subject is a mammal.

31. The method of claim 15, wherein the subject is a human.

32. The method of claim 15, wherein the compound is administered parenterally or orally.

33. The method of claim 15, wherein administering the compound or the pharmaceutical composition results in at least one less side effect as compared to administering the individual moiety alone.

34. The method of claim 15, wherein administering the compound or the pharmaceutical composition results in enhanced therapeutic activity as compared to administering the individual moiety alone.

35. A compound of formula (IV) or (V) or its pharmaceutically acceptable salts

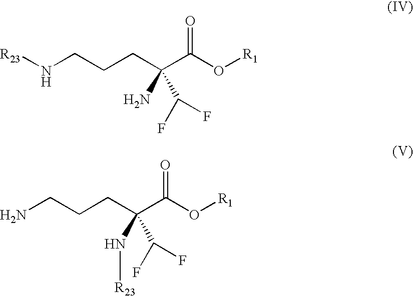

36. The compound of claim 35, wherein the phosphoramidate group is cleaved in vivo.

37. The compound of claim 35 further comprises a pharmaceutically acceptable carrier.

38. A method of treating or preventing cancer, the method comprising administering to a subject in need thereof a therapeutically effective amount of a compound of formula (IV) or (V) or its pharmaceutically acceptable salts.

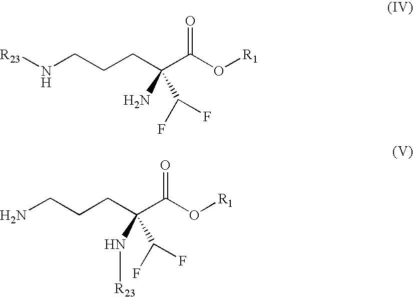

39. The method of claim 38, wherein the compound is administered orally.

40. The method of claim 38, wherein the compound is administered parenterally.

41. The method of claim 38, wherein the compound is administered in combination with an NSAID.

42. The method of claim 41, wherein the NSAID is selected from the group consisting of aspirin, aceclofenac, acemethacin, alclofenac, amoxiprin, ampyrone, azapropazone, benorylate, bromfenac, choline and magnesium salicylates, choline salicylate, celecoxib, clofezone, diclofenac potassium, diclofenac sodium, diclofenac sodium with misoprostol, diflunisal, droxicam, lornoxicam, meloxicam, tenoxicam, ethenzamide, etodolac, fenoprofen calcium, faislamine, flurbiprofen, flufenamic acid, ibuprofen, ibuproxam, indoprofen, alminoprofen, carprofen, dexibuprofen, dexketoprofen, fenbufen, flunoxaprofen, indomethacin, ketoprofen, ketorolac, kebuzone, loxoprofen, magnesium salicylate, meclofenamate sodium, metamizole, mofebutazone, oxyphenbutazone, phenazone, sulfinpyrazone, mefenamic acid, meloxicam, methyl salicylate, nabumetone, naproxen, naproxen sodium, nebumetone, oxaprozin, oxametacin, phenylbutazone, proglumetacin, piroxicam, pirprofen, suprofen, rofecoxib, salsalate, salicyl salicylate, salicylamide, sodium salicylate, sulindac, tiaprofenic acid, tolfenamic acid, tolmetin sodium, and valdecoxib.

43. The method of claim 41, wherein the NSAID is sulindac.

44. The method of claim 41, wherein the NSAID is aspirin.

45. The method of claim 41, wherein the NSAID is administered prior to, concomitant with, or subsequent to administering the compound of formula (II).

46. The method of claim 38, wherein the subject is a mammal.

47. The method of claim 38, wherein the subject is a human.

48. The method of claim 38, wherein the cancer is adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarcinoma, head and neck cancers, teratocarcinoma, or Waldenstrom's macroglobulinemia.

49. The method of claim 38, wherein the cancer is a Ki-ras-dependent cancer.

50. A composition for treating or preventing cancer, the composition comprising a salt of a mixture of eflornithine or an analog or derivative of eflornithine, and aspirin.

51. The composition of claim 50, wherein the eflornithine derivative is eflornithine ester.

52. A method of treating or preventing cancer, the method comprising administering to a subject in need thereof a therapeutically effective amount of a salt of a mixture of eflornithine or an analog or derivative of eflornithine, and aspirin.

53. The method of claim 52, wherein the eflornithine derivative is eflornithine ester.

54. The method of claim 52, wherein the salt is administered orally or parenterally.

55. The method of claim 52, wherein the subject is a human.

56. The method of claim 52, wherein the cancer is adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

57. The method of claim 52, wherein the cancer is a Ki-ras-dependent cancer.

58. The method of claim 52 further comprising administering to the subject at least one other therapeutic agent.

59. The method of claim 58, wherein the other therapeutic agent is an antitumor alkylating agent, antitumor antimetabolite, antitumor antibiotics, plant-derived antitumor agent, antitumor organoplatinum compound, antitumor campthotecin derivative, antitumor tyrosine kinase inhibitor, monoclonal antibody, interferon, biological response modifier, hormonal anti-tumor agent, angiogenesis inhibitor, differentiating agent, or a pharmaceutically acceptable salt thereof.

60. The method of claim 52, wherein the salt is administered in combination with surgery, radiation therapy, chemotherapy, gene therapy, RNA therapy, adjuvant therapy, immunotherapy, nanotherapy or a combination thereof.
